(19) United States
(12) Patent Application Publication
(10) Pub. No.: US 2025/0276056 A1
(43) Pub. Date: Sep. 4, 2025

(54) CYTOLYTIC T CELL IMMUNOTHERAPY FOR HIGHLY PATHOGENIC CORONAVIRUSES

(71) Applicants: Raphael Paul VISCIDI, Baltimore, MD (US); Charles J. PIVEN, Baltimore, MD (US)

(72) Inventors: Raphael Paul VISCIDI, Baltimore, MD (US); Charles J. PIVEN, Baltimore, MD (US)

(21) Appl. No.: 18/272,331

(22) PCT Filed: Jan. 14, 2022
(86) PCT No.: PCT/US2022/012434

§ 371 Date: Jul. 13, 2023

Related U.S. Application Data
(60) Provisional application No. 63/137,402, filed on Jan. 14, 2021.

(51) Int. Cl. A61K 39/215; A61K 39/00; A61P 31/14

(52) U.S. Cl. CPC A61K 39/215; A61P 31/14; A61K 2039/5258; A61K 2039/572; C12N 2710/14023; C12N 2710/14034; C12N 2770/20034

(57) ABSTRACT

Compositions and methods to induce cytolytic T lymphocytes (CD8+) response, that is, MHC class I restricted T cell responses, to pathogenic and common cold coronaviruses, including a delivery platform for antigens consisting of a polyionic papillomavirus virus-like particle (VLP), with contiguous, negatively charged amino acids flanked by a cysteine residue inserted in the HI loop of the papillomavirus L1 protein. Antigens to be paired with the VLP include fusion peptide/proteins derived from a pathogenic coronavirus, and from the genetically most closely related human coronaviruses that commonly circulate in human populations, with N-terminal or C-terminal amino acids consisting of contiguous, positively charged amino acids preceded and/or followed by a cysteine residue and a C-terminal proteolytic processing sequence (AAYY) to enhance presentation of MHC class I epitopes.

### BACKGROUND OF THE INVENTION

### Field of the Invention

The present invention relates to compositions and methods for inducing cellular immune responses to coronaviruses.

### Description of the Background

Coronaviruses (CoVs) are enveloped viruses with a single-strand, positive-sense RNA genome approximately 26-32 kilobases in size. SARS-CoV-2, like all coronaviruses, shares common features in the organization and expression of its genome; nonstructural proteins encoded by open reading frame (ORF) 1a/b, are followed by the principal structural proteins spike(S), envelope (E), membrane (M), and nucleocapsid (N) (Chen et al., 2020). The S glycoprotein mediates attachment to the host receptor. The S glycoprotein is cleaved by a host protease into two separate polypeptides designated S1 and S2. S1 makes up the receptor binding domain of the S protein and is the principal target of neutralizing antibodies, while S2 forms the stalk of the spike molecule. The M protein is the most abundant viral protein and participates in the formation of the core viral particle. The N protein is the only nucleocapsid protein and forms the portion of the core particle that interacts with the viral genome. The CoVs are separated into four genera based on phylogeny: α-CoV, β-CoV, γ-COV and δ-COV. Within the beta-CoV genus, four lineages (A, B, C, and D) are recognized.

Coronaviruses cause a large variety of diseases in animals. In humans, CoV infections primarily involve the upper respiratory tract and the gastrointestinal tract, and principally cause mild, self-limiting disease, such as the common cold. Two of these human coronaviruses are α-coronaviruses, HCoV-229E and HCoV-NL63, while the other two are β-coronaviruses, HCoV-OC43 and HCoV-HKU1. HCoV-229E and HCoV-OC43 were isolated nearly 50 years ago, while HCoV-NL63 and HCoV-HKU1 have only recently been identified. These viruses are endemic in the human populations, causing 15-30% of respiratory tract infections each year. Seroprevalence studies suggest that exposure to these viruses is nearly universal in humans (Severance et al., 2008). The existence of additional human coronaviruses is plausible but unknown. The first highly pathogenic human coronavirus was SARS-COV, a β-coronavirus. It was identified as the causative agent of the Severe Acute Respiratory Syndrome (SARS) outbreak that occurred in 2002-2003 in the Guangdong Province of China. A second novel, highly pathogenic human CoV emerged in the Middle East in 2012. This virus, named Middle East Respiratory Syndrome-CoV (MERS-COV), is also a β-coronavirus and was found to be the causative agent of highly pathogenic respiratory tract infections in Saudi Arabia and other countries in the Middle East. The third and most recent highly pathogenic human coronavirus, SARS-CoV-2, first appeared in China in late 2019, is also a β-coronavirus, and is currently responsible for an ongoing pandemic. The highly pathogenic coronaviruses are believed to circulate in zoonotic reservoirs, principally in bat species, with occasional spillover into the susceptible human population, possibly via an intermediate host species. The occurrence of 3 cross species transmission events during the past 17 years raises the prospect that similar viruses may emerge in the future. The defining feature of highly pathogenic coronaviruses is their ability to cause serious morbidity and mortality in infected individuals, with crude estimates of 2.3%, 9.5%, and 34% for SARS-CoV-2, SARS-CoV-1 and MERS, respectively (Petrosillo et al., 2020). The commonly circulating human coronaviruses rarely if ever cause serious morbidity or mortality.

Treatment of pathogenic coronaviruses is primarily supportive. Despite ongoing efforts, there are no highly active anti-viral drugs.

### SUMMARY OF THE INVENTION

The present invention relates to treatment and prevention of disease caused by the highly pathogenic coronaviruses, SARS-CoV-1, MERS and SARS-CoV-2, and other related pathogenic coronaviruses.

The approach of the present invention to treatment of coronaviruses is an immunotherapy which harnesses the natural immune defenses of the body. For clearance of an existing viral infection the paradigm of immune defense is the MHC class 1-restricted CD8+ cytotoxic T lymphocyte, which has the ability to destroy and clear virally infected cells. However, it is problematic whether induction of T cells directed against an infecting virus can clear that infection before the host is overwhelmed, because a maximal response to immunization typically requires weeks, perhaps months to achieve. Therefore, in the case of highly pathogenic coronaviruses, a strategy is needed to rapidly mobilize an efficacious anti-viral cytolytic T cell response to control or clear acute infection. The proposed approach is to stimulate cross reactive memory T cells. Memory T cells, which are more responsive to stimulation than naïve cells, can be clonally expanded very rapidly. Recent studies have identified cross-reactive T cells that recognize SARS-CoV-2 antigens in blood samples obtained prior to the appearance of the virus in 2019 (Grifoni et al., 2020; Weiskopf et al., 2020; Braun et al., 2020; Le et al., 2020). Since the common cold coronaviruses, OC43 and HKU1, share amino acid similarities with SARS-CoV-2, these cross-reactive T cells may be induced by prior infections with common cold human coronaviruses. In fact, a recent study that mapped the epitopes recognized by cross reactive T cells demonstrated that pre-existing memory CD4+ T cells are cross reactive with SARS-CoV-2 and common cold coronaviruses (Mateus et al., 2020). The detailed molecular and structural basis for cross reactive T cells is poorly understood but the current paradigm of immune recognition predicts the requirement for highly conserved amino acid sequences within MHC class I-restricted T cell epitopes (contiguous sequences 8-13 amino acids in length) that are shared by SARS-CoV-2 and the common cold coronaviruses. Alignments of SARS-CoV-2 amino acid sequences with homologous regions of known common human coronaviruses show only limited regions of high amino acid identity in putative T cell epitopes. However, the notion that cross reactivity requires substantial amino acid identity between epitopes from heterologous viruses is based on the clonal selection theory of immune recognition, which postulates individual lymphocytes are specific for a single antigen, one clone-one specificity, and requires close amino acid identity between cross reactive epitopes. Several scientists have called this theory into question and proposed a theory of T cell immune recognition that postulates T cells recognize multiple specificities, one clone-millions of specificities (Mason, 1998; Wilson et al., 2004; Kersh and Allen, 1996; Sewell, 2012). The theory is grounded in the mathematic consideration that there are only 1012 T cells in humans and <10⁸ distinct T cell receptors in the human naïve T cell pool, while the theoretical limit of possible peptides of 20 amino acids that can bind to MHC molecules is vast (>10¹⁵) and likely even greater if peptides that contain post-translational modifications are considered. This theory of T cell cross reactivity leads to a novel approach to both treat and prevent highly pathogenic coronaviruses. Simultaneous induction of T cell responses to a highly pathogenic coronavirus and to one or more genetically related common human coronaviruses to which the majority of individuals have prior exposure and thus have memory T cells will generate two categories of cellular immunity, therapeutic and prophylactic. The strategy will recall cross-reactive partially or fully protective memory T cells and induce naïve T cells specific for the highly pathogenic coronavirus. The dual action of these two classes of T cell response will be therapeutic by rapidly controlling and eradicating the acute highly pathogenic coronavirus infection in the host, preventing severe symptoms and death, and at the same time be prophylactic by providing long term protection against future infection with the highly pathogenic coronavirus.

The present invention further relates to compositions and methods to induce cytolytic T lymphocytes (CD8+) response, that is, MHC class I restricted T cell responses, to pathogenic and common cold coronaviruses.

The invention relates to a delivery platform for antigens consisting of a polyionic papillomavirus virus-like particle (VLP), wherein contiguous, negatively charged amino acids flanked by a cysteine residue are inserted in the HI loop of the papillomavirus L1 protein.

The invention further relates to antigens to be paired with the VLP comprising fusion peptide/proteins with N-terminal or C-terminal amino acids consisting of contiguous, positively charged amino acids preceded and/or followed by a cysteine residue, here after designated the TAG. The invention further relates to the TAG having a C-terminal proteolytic processing sequence (AAYY) to enhance presentation of MHC class I epitopes.

In particular embodiments, the invention relates to antigens that are derived from a pathogenic coronavirus, and from the genetically most closely related human coronaviruses that commonly circulate in human populations. The invention further relates to the choice of antigens for induction of a cytolytic T cell response based on the abundance of expression of the viral protein in virally infected cells, the density and position of predicted MHC class I-restricted T cell epitopes in the protein, and empirical studies identifying cytolytic T cells directed toward specific viral proteins. The invention further relates to the choice of antigens comprising two separate and distinct classes of targets that are derived from viral structural proteins and viral non-structural proteins, respectively. Since the first proteins expressed in virally infected cells are non-structural viral proteins, targeting these proteins provides for an efficacious cellular immune response at the earliest stage of viral infection. The invention further relates to the choice of antigens of the common nonpathogenic human coronavirus based on the above criteria and the additional criterion that the antigen encompasses regions of the viral protein that share at least 40% identity to the corresponding viral protein of the pathogenic coronavirus. In particular embodiments of VLP-antigen compositions, the antigens are either short peptides 25-30 amino acids in length, extended peptides approximately 45-55 amino acids in length, short proteins approximately 110-140 amino acids in length, or full-length amino acid sequences of target antigens.

In a particular embodiment, the pathogenic coronavirus is SARS-CoV-2 and the genetically related human coronaviruses are OC43 and HKU1. In a particular embodiment the antigens of SARS-CoV-2 are derived from the following viral structural proteins, the membrane protein (M), the nucleocapsid protein (N), ORF3a, ORF7a, and the S2 region of the spike(S) envelope protein and the antigens of the non-structural proteins are derived from the nsp6, nsp7 and nsp12. In a particular embodiment, the antigens of the structural proteins of OC43 and HKU1 are derived from the M and N proteins and the S2 region of the S protein, and the antigens of the non-structural proteins are derived from the nsp3, nsp4, nsp6, nsp7 and nsp12 proteins of OC43, and variable regions of HKU1 with respect to OC43 from the same viral proteins.

The invention also relates to the method of preparing polyionic papillomavirus VLPs paired with coronavirus antigens. The invention further relates to the method of delivery of the VLP-antigen composition by the intravenous, intramuscular or intradermal route. In a particular embodiment, the VLP-antigen composition is delivered intranasally or by inhalation to stimulate lung and nasopharyngeal tissue resident memory T cells and generate cytotoxic T lymphocytes that preferentially traffic to sites into the respiratory tract.

### BRIEF DESCRIPTION OF THE DRAWINGS

FIG. 1 shows alignment of bovine papillomavirus (BPV) type 1 L1 protein with the L1 protein of the closely related BPV isolate NY8385: HI loop is aa344-357.

FIG. 2 shows alignment of M proteins of OC43 and SARS-CoV-2.

FIG. 3 shows alignment of M proteins of OC43 and HKU1.

FIGS. 4A and 4B show alignments of antigenic regions of N proteins of OC43 and SARS-CoV-2.

FIGS. 5A, 5B and 5C show alignment of antigenic regions of N proteins of OC43 and HKU1.

FIGS. 6A and 6B show alignments of antigenic regions of S proteins of OC43 and SARS-CoV-2 within the S2 region.

FIG. 7 shows alignment of S proteins of HKU1 and OC43 within a variable antigenic region.

FIGS. 8A, 8B and 8C show alignment of antigenic regions of ORF1ab of OC43 with SARS-CoV-2 nsp3 protein.

FIGS. 9A, 9B and 9C show Alignment of antigenic regions of ORF1ab of OC43 with SARS-CoV-2 nsp4 protein.

FIGS. 10A and 10B show alignment of antigenic regions of ORF1ab of OC43 with SARS-CoV-2 nsp6 protein.

FIG. 11 shows alignment of antigenic region of ORF1ab of OC43 with SARS-CoV-2 nsp7 protein.

FIG. 12 shows the sequence of Bovine Papillomavirus type 1 capsid protein L1.

FIG. 13A shows alignment of a first set of antigenic regions of ORF1ab of OC43 with SARS-CoV-2 nsp12 protein.

FIG. 13B shows alignment of a second set of antigenic regions of ORF1ab of OC43 with SARS-CoV-2 nsp12 protein.

FIG. 13C shows alignment of a third set of antigenic regions of ORF1ab of OC43 with SARS-CoV-2 nsp12 protein.

### DETAILED DESCRIPTION

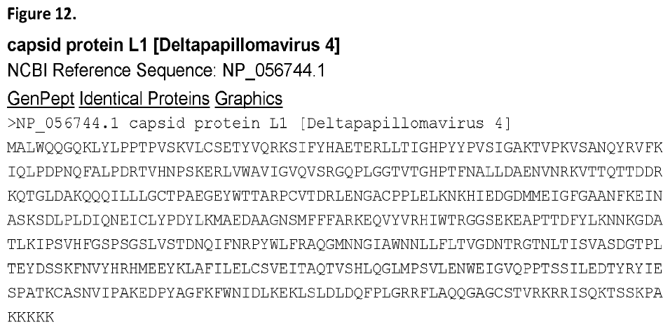

The present invention provides compositions and methods relating to genetically engineered papillomavirus L1 virus-like particles (VLPs) comprising negatively charged amino acid sequences and a cysteine residue in the HI loop of the L1 protein, hereafter referred to as polyionic VLPs. The invention further relates to an L1 protein from any papillomavirus species, including human, bovine, equine, murine, ovine, porcine, cervine, canine, feline, or leporine. In one embodiment, the L1 papillomavirus protein is from bovine papillomavirus type 1 (BPV1) (FIG. 12).

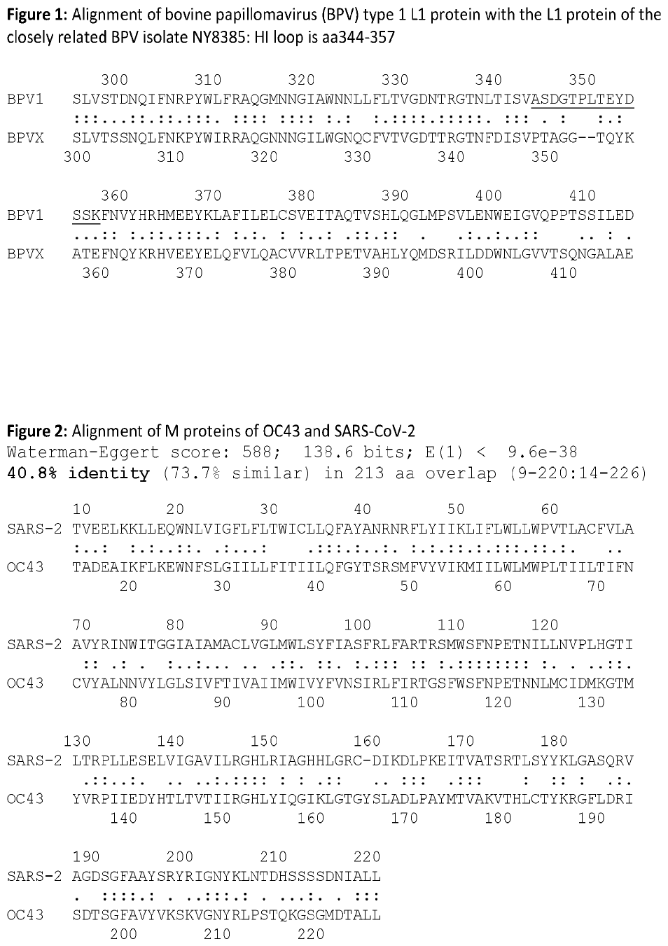

In particular embodiments, the HI loop of the genetically engineered BPV1 L1 protein comprises 4 to 10 contiguous, negatively charged amino acids, flanked by a cysteine residue at the N- or C-terminus or both termini. The HI loop of BPV1 L1 is here defined as amino acid positions 344 to 357, with a span of 14 amino acids and the tip defined as the proline (P) residue at position 349 (FIG. 1). The negatively charged amino acids of the VLPs of the present invention can be glutamic acid, aspartic acid, or both. In various embodiments, the amino acid sequence of 5 to 12 amino acids in length (4-10 contiguous, negatively charged amino acids and one or two flanking cysteine residues) may be inserted in the HI loop, and replace none, or one, or more than one of the native amino acids. In specific exemplary embodiments, the amino acid sequence inserted in the HI loop is 9 residues in length, comprising 8 negatively charged amino acids, glutamic acid or aspartic acid or alternating aspartic and glutamic acids, and a C- or N-terminal cysteine residue, and replaces the 9 native amino acids at positions 347-355 of the HI loop of the bovine papillomavirus L1 protein (Table 1, inserts 1-4). In other specific exemplary embodiments, the amino acid sequence inserted in the HI loop is 5, 6, 7, 8, 10, or 11 amino acids in length, comprising 4, 5, 6, 7, 9, or 10 glutamic acids, respectively, and a C-terminal cysteine residue. The respective inserts replace the equivalent number of native amino acids at positions, 347-351, 347-352, 347-353, 347-354, 346-355 or 345-355, respectively, of the HI loop of the bovine papillomavirus L1 protein (Table 1 insert 5-10). In a specific exemplary embodiment, the amino acid sequence inserted in the HI loop is 9 residues in length, comprises 8 negatively charged glutamic acids and a C- and N-terminal cysteine residue, and replaces the 10 native amino acids at positions 346-355 of the HI loop of the bovine papillomavirus L1 protein (Table 1, insert 11). In other various embodiments the negatively charged amino acids and cysteine(s) may be inserted in the HI loop and replace fewer native amino acids than the number comprising the insert. In specific exemplary embodiments a 9-amino acid sequence, comprising 8 glutamic acids and a C-terminal cysteine residue replaces 7, 5 or 3 native amino acids at positions 348-354, or 348-352, or 348-350, respectively, of the HI loop of the bovine papillomavirus L1 protein (Table 1, inserts 12-14). In other various embodiments the negatively charged amino acids and cysteine(s) may be inserted in the HI loop and replace up to 2 more native amino acids than the number comprising the insert. In a specific exemplary embodiment, a 9-amino acid sequence, comprising 8 glutamic acids and a C-terminal cysteine residue, replaces the 11 native amino acids at position 346-356 of the HI loop of the bovine papillomavirus L1 protein (Table 1, insert 15). In various embodiments, the glutamic acid-cysteine amino acid sequence inserted in the HI loop may replace the proline at the putative tip of the loop or may be inserted between the proline residue and the immediate C-terminal leucine residue without removing any native amino acids. In some embodiments, the inserted amino acid replacing proline or located between the proline and leucine residues may be flanked by a glycine-serine-serine-glycine (GSSG) linker amino acid sequence. In specific exemplary embodiments, the amino acid sequence inserted in the HI loop comprises 8 glutamic acids and a C-terminal cysteine residue, with or without flanking GSSG amino acids, and either replaces the proline at position 349 or is located between amino acid positions 349 and 350 amino acid of the HI loop of the bovine papillomavirus L1 protein (Table 1, inserts 16-19).

The invention further relates to antigens to be linked to the VLP, comprising fusion peptide/proteins with N-terminal or C-terminal amino acids consisting of 4-10 contiguous, positively charged amino acids preceded by and/or followed by a cysteine residue, here after designated the TAG. The TAG allows the antigen to be attached to the polyionic papillomavirus VLP by the combined action of electrostatic interactions and an oxidization reduction reaction between cysteine residues on the VLP and the cysteine residue in the TAG. The invention further relates to the TAG having a C-terminal proteolytic processing sequence (AAYY) to enhance presentation of MHC class I epitopes. Where the TAG is appended to the C-terminus of the antigen, the proteolytic processing sequence (AAYY) is placed at the N-terminus of the peptide/protein antigen.

In specific exemplary embodiments the TAG comprises the group of positively charged amino acids, arginine (R), lysine (K) or histidine (H), and the sequence is 8 amino acids in length, (Table 2, Tag-1, -2, -3). In other specific exemplary embodiments, the TAG comprises a repeating motif of RKHRKHRK, 8 amino acids in length (Table 2, TAG-4). In other specific exemplary embodiments, the TAG comprises an amino acid sequence of 4, 5, 6, or 7 contiguous arginines followed by a cysteine residue (Table 2, TAG-5, -6, -7, and -8) or an amino acid sequence of 9 or 10 arginines followed by a cysteine residue (Table 2 TAG-9 and -10). In other specific exemplary embodiments, the TAG consists of 8 positively arginines preceded by a cysteine residue or flanked at the N and C terminus by cysteine residues (Table 2, TAG-11 and -12)

The invention further relates to the composition of polyionic papillomavirus VLPs and target antigens with a TAG sequence for the purpose of inducing cytolytic (MHC class I restricted) T cell responses. In specific embodiments, the invention relates to coronavirus antigens linked to polyionic VLPs for the purpose of inducing cytolytic T cell responses to common cold coronaviruses and pathogenic coronaviruses.

The entire proteome of a virus can be a target of T cell responses. However, structural proteins are particularly effective targets for antigen-specific T cell responses because they are the most abundantly expressed viral proteins in infected cells, thus allowing for efficient presentation to MHC molecules. Empirical studies of cellular immune responses to coronaviruses also support the importance of structural proteins as principal targets of the cellular immune response (reviewed in (Liu et al., 2017). Cytolytic T cell responses are directed toward MHC class I restricted T cell epitopes. These epitopes are most commonly 9 amino acids in length and can range from 8-13 amino acids in length. The genetic heterogeneity of MHC class I alleles in humans and other mammals allows for numerous possible epitopes within a viral protein. In order to encompass the universe of possible epitopes, an antigen needs to be the full-length amino acid sequence of a target protein or sets of shorter fragments of the antigen that together include the entire amino acid sequence. The preferred length of antigenic fragments linked to polyionic VLPS is an empirical function of efficiency of epitope presentation for induction of a T cell response via the alternative antigen presentation pathway. In addition, manufacturing considerations may influence the preferred choice of length of an antigen.

The preferred embodiment for a polyionic papillomavirus VLP composition of matter for the induction of cytotoxic T cell response to SARS-CoV-2 comprises antigens from the membrane (M), nucleocapsid (N), S2 region of the spike(S), ORF3a, and ORF7a structural proteins and the nsp6, nsp7 and nsp12 nonstructural proteins.

The M protein is 222 amino acids in length and the antigenic region spans aa6-221. The N protein is 419 amino acids in length. Based on empirical studies and predictive algorithms for MHC class I-restricted T cell epitope the antigenic region is aa51-369. The Spike(S) protein is 1270 amino acids in length and comprises two regions, S1 (aa1-661) containing the receptor binding domain (aa330-583) and S2 (aa662-1270). T cell epitopes are widely distributed across the S protein. The S1 region contains the majority of virion surface exposed amino acids and is the principal target of humoral immune responses, including neutralizing antibodies. The region is preferably excluded from a vaccine intended to induce cellular immunity to avoid unintended induction of antibody responses with possible deleterious effects, such as induction of antibodies mediating antibody dependent enhancement (ADE). ORF3a is 275 amino acids in length. Empirical studies support the choice of ORF3a as a target antigen for T cell response. Prediction algorithms (MHC-NP: prediction of peptides naturally processed by MHC, developed by Sébastien Giguère, Alexandre Drouin, Alexandre Lacoste, Mario Marchand, Jacques Corbeil and François Laviolette; http://tools.iedb.org/mhcnp/) show that the C terminus, as compared to the N terminus, has a greater density of putative MHC class I restricted epitopes for 5 representative alleles (67 spanning the C-terminal 171 amino acids versus 20 spanning the 104 N-terminal amino acids). Empirical studies support choice of ORF7a, a protein of 121 amino acids, as a target of cytolytic T cell responses. The ORF1ab of SARS-CoV-2 is a polyprotein of 7096 amino acids. The polyprotein is proteolytically processed within virally infected cells to yield multiple non-structural proteins that serve diverse functions in the viral life cycle. The proteins are present in low abundance in virally infected cells and for this reason are not major targets of cellular immune responses, but recent studies have shown that cytolytic T cell responses to several nsp proteins can be detected in the blood of SARS-CoV-2 infected patients and also in blood obtained from some healthy blood donors in the pre-COVID19 era (Grifoni et al., 2020; Le et al., 2020). Because these proteins are the first to be expressed in infected cells they are attractive targets for cytolytic T cells. Rapid killing of these virally infected cells can prevent the cell from producing infectious virus. In terms of frequency of antigen specific T cells and percentage of responding subjects, the principal targets of CD8+ T cell responses are the nsp6, nsp7 and nsp12 proteins.

The amino acid length of antigens linked to polyionic VLPs can range from a defined epitope of 8-14 amino acids or longer amino acid sequences up to the full-length amino acid sequence of the antigenic region of a viral protein or fusions of several viral proteins. In exemplary embodiments, the antigens are short peptides approximately 25-30 amino acids in length, extended peptides approximately 45-55 amino acids in length, short proteins approximately 110-140 amino acids in length, or the full-length amino acid sequence of the antigenic region of a targeted viral protein. For antigenic fragments shorter than the full-length amino acid sequence of the antigenic region, the specific embodiment comprises a set of antigens that includes all the amino acids of the antigenic region of the target viral protein. Antigens for the M protein of SARS-CoV-2, embodied as short peptides, extended peptides, short proteins, or the entire amino acid sequence of the antigenic region, are shown in Table 3. Comparable antigens for the N protein of SARS-CoV-2 are shown in Table 4. Comparably designed antigens for the S2, ORF3a, and ORF7a structural proteins of SARS-CoV-2 are shown in Tables 5-7. Antigens for the nonstructural proteins nsp6 and nsp7 are shown in Table 8 and antigens for the nsp12 protein are shown in Table 9 and 10. Short and extended peptides are overlapping by 10 amino acids and short protein antigens are overlapping by 11 amino acids. In the final formulation, peptide, protein, or full-length antigens have a TAG, as described above.

The preferred embodiment for a polyionic papillomavirus VLP composition of matter to stimulate memory T cells induced by prior exposure to common cold coronaviruses comprises antigens derived from both structural and nonstructural proteins. The multiple specificities theory of T cell recognition does not define at the molecular level the structural basis for T cell cross reactivity. We define herein viral proteins or sub regions of viral proteins containing cross-reactive T cell epitopes as those with an average amino acid identity of >40% across homologous amino acid sequences of a common cold coronavirus and a specific pathogenic coronavirus.

In particular embodiments, the common coronaviruses are OC43 and HKU1, and the antigens for induction of cross-reactive T cell responses are from the M, N and S2 structural proteins and the nsp3, nsp4, nsp6, nsp7 and nsp12 nonstructural proteins. Where the amino acids sequences of OC43 and HKU1 share on average>40-80% identify across homologous amino acids of the target antigen, only the amino acid sequences of OC43 are used as the target antigens. In other particular embodiments, the amino acid sequences of HKU1 are the antigens.

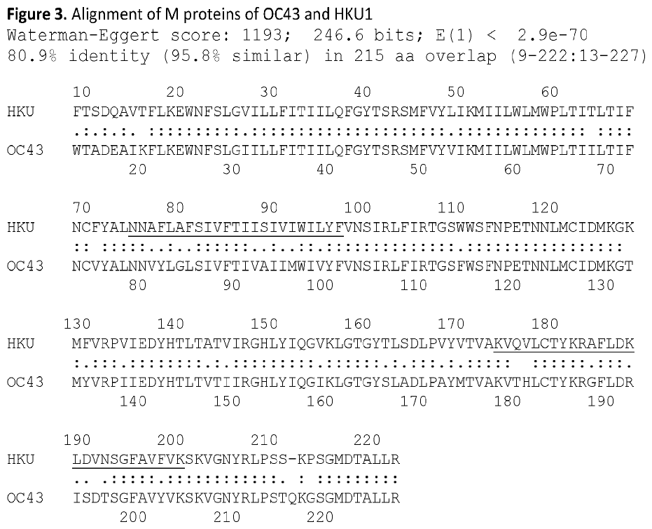

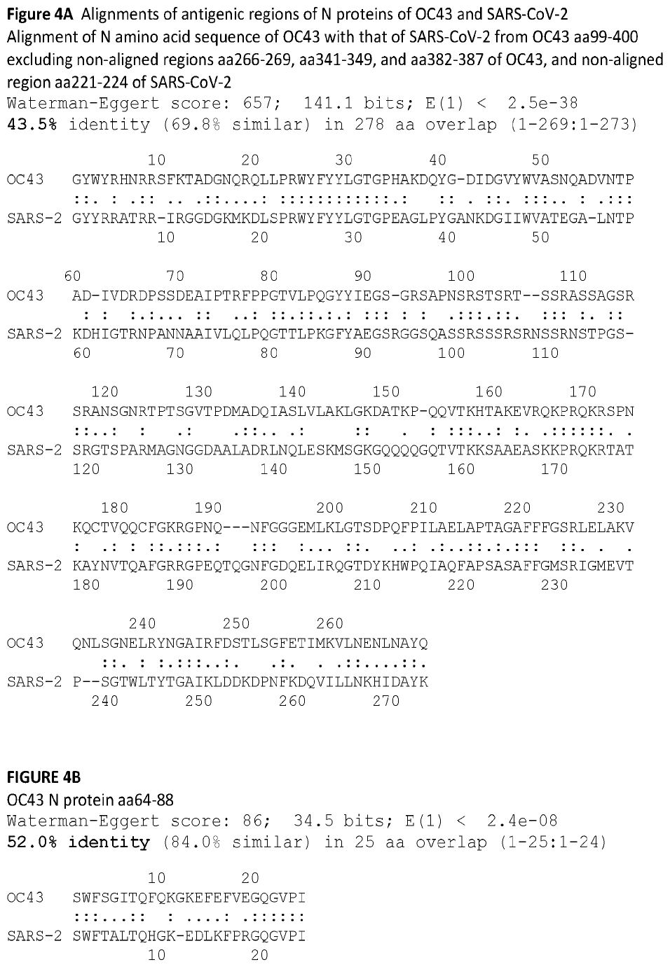

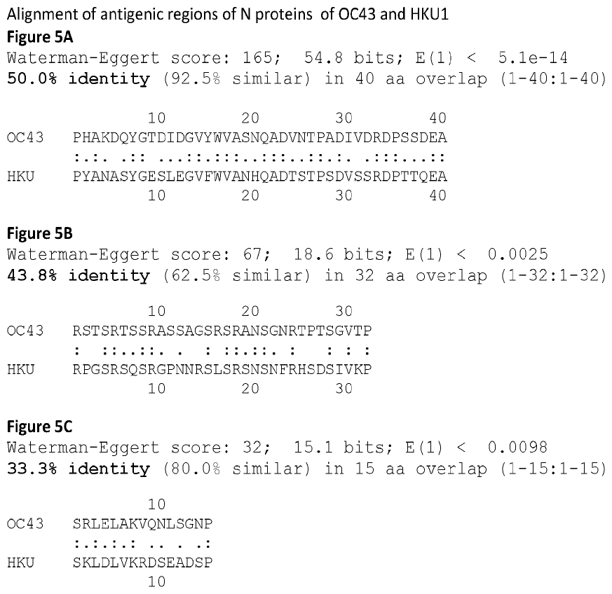

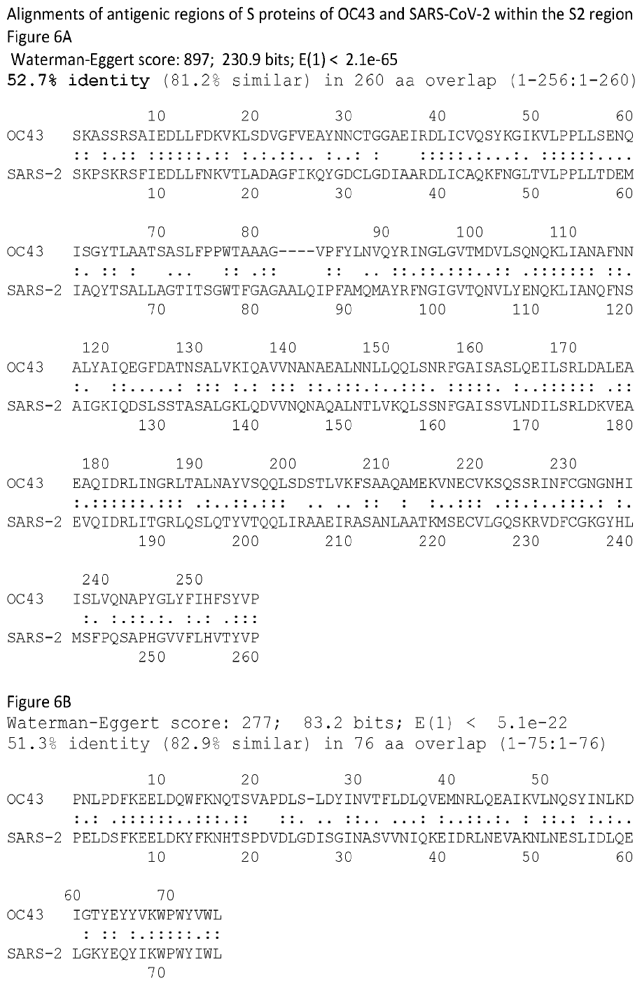

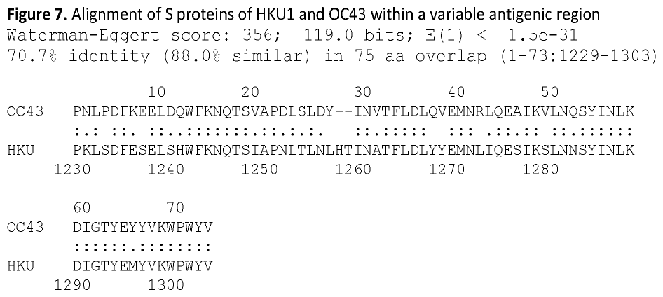

The M protein of OC43 is 230 aa in length and shares overall identity of 40.8% with the M protein of SARS-CoV-2 within the region of aa14-226 (FIG. 2). Alignment of OC43 M protein with that of HKU1 shows no significant areas of variability (amino acid identity<80%) (FIG. 3). The N protein of OC43 is 448 aa in length and on average shares 36% amino acid identity with the N protein of SARS-CoV-2. The antigenic region of OC43 from aa99-400, excluding non-aligned regions aa266-269, aa341-349, and aa382-387, shares 43.5% identity with the homologous region of the N protein of SARS-CoV-2, after excluding aa221-224 that do not align with the amino acid sequence of the OC43 N protein (FIG. 4A). The N-terminus amino acids of the OC43 N protein at positions aa64-88 share 52% identity with the corresponding region of N of SARS-CoV-2 (FIG. 4B). Alignment of OC43 N protein with that of HKU1 shows 3 antigenic regions with low levels of amino acid identity between the two viral amino acid sequences, ranging from 33%-50%. (FIGS. 5A, 5B, and 5C). The S protein of OC43 is 1353 amino acids and composed of 2 sub-regions, S1 and S2. The S1 extends from aa 1-789 and shows low amino acid identity (24%) with the S1 region of SARS-CoV-2 in the aligned region between aa70-552. In contrast, the S2 region of the viruses contains two antigenic regions, aa898-1153 and aa1228-1302, that share an average of 52.72% and 51.3% amino acid identity, respectively (FIG. 6). Alignment of the homologous region of HKU1 with aa898-1153 of OC43 showed overall identity of 83.5% without regions of high variability, while the antigenic region of OC43 between aa1228-1303 shares 70.7% identity with the homologous S2 region of HKU1 (FIG. 7).

In exemplary embodiments, the antigens are short peptides approximately 25-30 amino acids in length, extended peptides approximately 45-55 amino acids in length, short proteins approximately 110-140 amino acids in length, or the full-length amino acid sequence of the antigenic region of a targeted viral protein. For antigenic fragments shorter than the full-length amino acid sequence of the antigenic region, the specific embodiment comprises a set of antigens that includes all the amino acids of the antigenic region of the target viral protein. Antigens for the M protein of OC43 and variable regions of HKU1, embodied as short peptides, extended peptides, short proteins, or the entire amino acid sequence of the antigenic region, are shown in Table 11. Comparable antigens for the N protein of OC43 and variable regions of HKU1 are shown in Table 12, and antigens for the S2 protein of OC43 and variable regions of HKU1 in Table 13. Short and extended peptides are overlapping by 10 amino acids and short protein antigens are overlapping by 11 amino acids. In the final formulation peptide, protein or full-length amino acid sequences include a TAG, as described above.

The ORF1ab of OC43 coronaviruses encodes a polyprotein of 7095 amino acids. The polyprotein is proteolytically processed within virally infected cells to yield multiple non-structural proteins that serve diverse functions in the viral life cycle. In terms of frequency of antigen specific T cells and percentage of responding subjects, the principal targets of CD8+ T cell responses are the nsp3, nsp4, nsp6, nsp7 and nsp12 proteins. The choice of antigens from these nonstructural proteins is also based on the additional consideration of an average of >40% identity between amino acid sequences of common cold viruses and SARS-CoV-2.

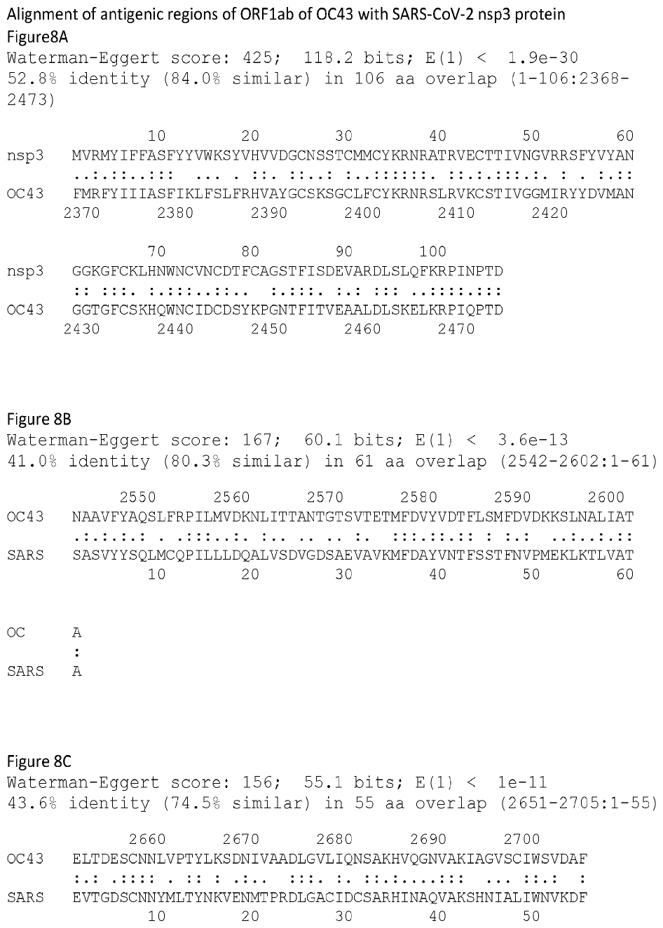

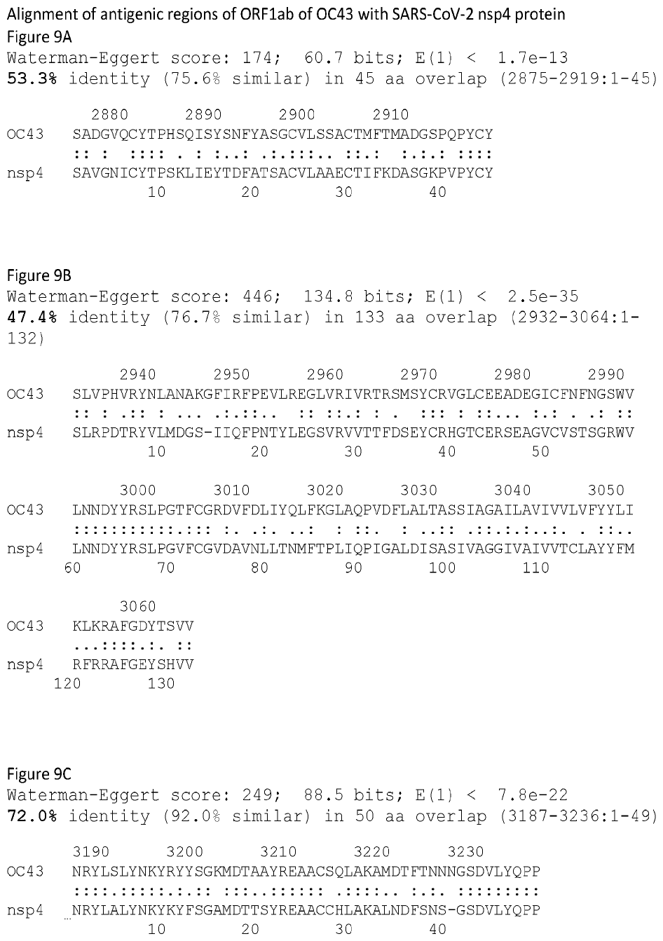

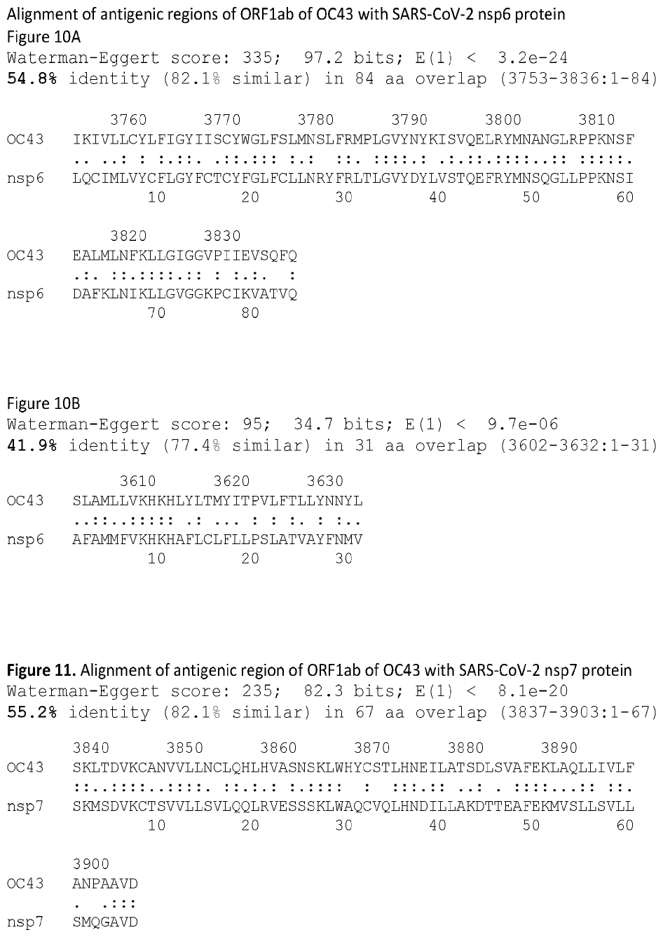

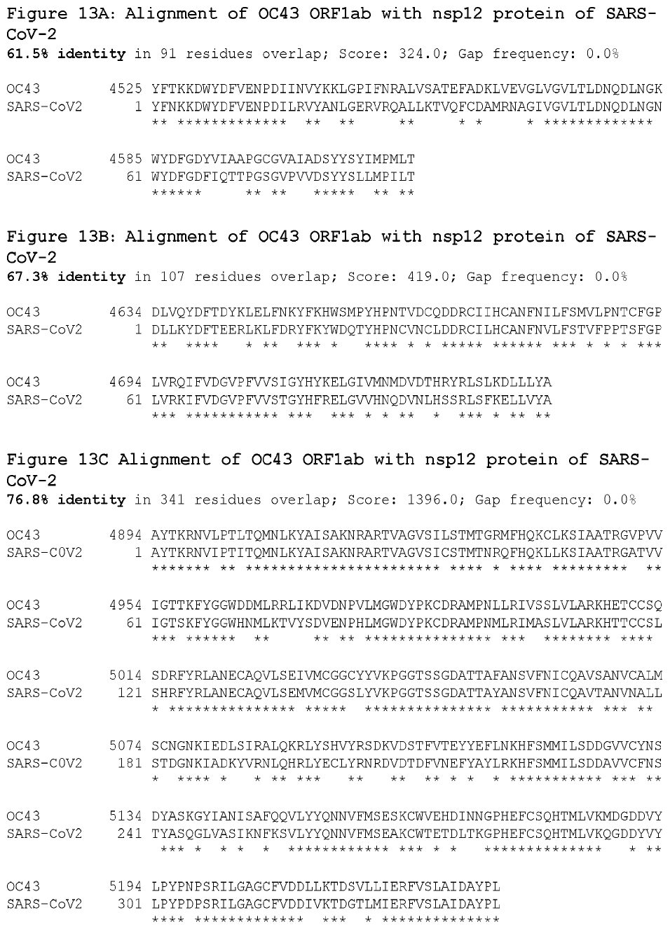

The nsp3 protein is 1,945 amino acids in length. The overall identity between SARS-CoV-2 and OC43 nsp3 amino acids sequences is 26%. However, the C-terminal region of 384 amino acids shares 39% identity and contains within it 3 regions of continuous amino acids with 52.8%, 41%, and 43.6% identity between the amino acid sequence of OC43 and the homologous amino acid sequence of SARS-CoV-2 (FIG. 8A-C). The nsp 4 protein is 500 amino acids in length; the nsp4 proteins of SARS-CoV-2 and OC43 share 42% amino acid identity. Three sub-regions in the mid portion or C terminus of the nsp4 protein, which are 45, 133 and 50 amino acids in length, share 53.3%, 47.4% and 72% amino acid identity, respectively, between homologous amino acid sequences of SARS-CoV-2 and OC43 (FIG. 9A-C). The nsp6 protein is 290 amino acids in length and the homologous amino acid sequences of SARS-CoV-2 and OC43 share 30.5% identity. The C-terminal 84 amino acids share 54.8% identity and a 31 amino acid region at the N-terminus shares 41.9% identity (FIG. 10A-B). The nsp7 protein is 83 amino acids in length and the amino acid sequences of the SARS-CoV-2 and OC43 nsp7 share 46% identity. Excluding the C-terminal 13 amino acids of SARS-CoV-2, the amino acid identity of SARS-CoV-2 and OC43 nsp7 proteins is 55.2% (FIG. 11). The nsp12 protein is 932 amino acids in length. An empirical study of antigen specific cellular immune responses in SARS-CoV-2 infected patients located the majority of T cell epitopes in the regions encompassed by aa 125-375 and aa 520-920 of the protein (Grifoni et al, 2021) . . . . Two amino acid sequences within OC43 ORF1ab share 61.5% and 67% identity with corresponding amino acids within the aa125-275 fragment of the SARS-CoV-2 nsp12 protein (FIGS. 13A-B) and 341 aa of OC43 ORF1ab shares 76.8% identity with corresponding amino acids within the aa520-920 fragment of the SARS-CoV-2 nsp12 protein (FIG. 13C). The nonstructural proteins of OC43 and HKU1 share ˜90% identity and have no extended regions of amino acid variability (identity<80%).

In exemplary embodiments, the antigens are short peptides approximately 25-30 amino acids in length, extended peptides approximately 45-55 amino acids in length, short proteins approximately 110-140 amino acids in length, or the full-length amino acid sequence of the antigenic region of a targeted viral protein. For antigenic fragments shorter than the full-length amino acid sequence of the antigenic region, the specific embodiment comprises a set of antigens that includes all the amino acids of the antigenic region of the target viral protein. Antigens for the nsp3 and nsp4 proteins, embodied as short peptides, extended peptides, short proteins, or the entire amino acid sequence of the antigenic region are shown in Table 14. Comparably designed antigens for the nsp6 and nsp7 proteins of OC43 are shown in Table 15 and for nsp12 in Tables 16 and 17. Short and extended peptides are overlapping by 10 amino acids and short protein antigens are overlapping by 11 amino acids. In the final formulation peptide, protein or full-length amino acid sequences include a TAG, as described above.

### EXAMPLES

### Example 1: Generation of Recombinant Baculoviruses with Genetically Engineered BPV L1 Genes

The entire open reading frame (ORF) of BPV L1 with a Kozak consensus and unique restrictions sites at each end (EcoR1/Not1) is artificially engineered by PCR-based gene synthesis and cloned in a pUC18 vector. The entire ORF is codon-modified using Drosophila melanogaster preferred codons, for efficient expression in insect cells. The ORF contains insertion of peptides with aspartic or glutamic acid residues and a cysteine residue and inserted into the HI loop as described in Table 1, and designated insert-1 to insert-19.

The modified BPV L1 genes are subcloned between the EcoR1/Not1 sites of the pORB baculovirus transfer vector. The transfer vectors are co-transfected with a linear baculovirus DNA in Spodoptera frugiperda sf9 cells using a preferred commercially available transfection reagent, as suggested by the manufacturer. Five days post-transfection, the recovered recombinant baculoviruses are further amplified by large scale infections of sf9 cells. Small scale infections to confirm expression of the modified L1 proteins are conducted with 2×10⁶ Trichoplusia ni (High Five) cells, growing in 6-well plates and infected with serial dilutions of Baculovirus stocks. 72 hrs post-infection, the cells are lysed in 500 μl of RIPA buffer and the clarified lysates are subjected to SDS-PAGE analysis to detect overexpression of a protein of the expected molecular weight of 55 kDa.

### Example 2: Production of Polyionic VLPs from Recombinant Baculoviruses

For production of VLPs, approximately 2× 109 Trichoplusia ni (High Five) cells growing in spinner flasks are infected with a pre-determined amount of a high-titer recombinant baculovirus stock in 500 ml of TNM-FH/10% FBS. After 96 h of incubation at 27° C., the cells are harvested, and collected by centrifugation at 2,000 rpm (Sorvall FH18/250 rotor) for 5 min. Cell pellets are resuspended in extraction buffer (20 mM phosphate buffer, pH 6.5, 1 M NaCl, 0.1 mM CaCl₂, 50 μm FeCl₂) containing protease inhibitors (Roche Complete ULTRA, 1 tablet per 10 ml) and subjected to 5 cycles of thawing at 37° C. and freezing in a −80° C. ethanol bath. The lysate is spun 1 h at 8000 rpm to remove baculovirus particles. The clarified lysate is extracted for 10 min with an equal volume of Vertrel DF (Fisher Scientific). The aqueous layer is loaded onto a 40% sucrose cushion and centrifuged in a SW32Ti rotor at 32,000 rpm for 1.5 h. The sucrose pellet is resuspended in 20 mM phosphate pH 8.0, 0.5 M NaCl, 5 mM MgCl₂, and incubated 30 min at 37° C. with 250 U/ml of Salt Active Nuclease (Arcticzymes). After dialysis in 20 mM phosphate pH 6.5, 0.5 M NaCl, the VLP solution is adjusted to 0.01% Tween 80, 0.05% carboxymethyl cellulose, 50 μM FeCl₂ and stored at 4° C. Purity is assessed by SDS-PAGE gel analysis and protein concentration is measured by Bradford dye method and uv spectroscopy. To facilitate direct visualization of VLPs, an aliquot of diluted particles is placed on 300-mesh formvar/carbon-coated copper grids, negatively stained with 2% phosphotungstic acid (pH=7.0) and examined by transmission electron microscopy (TEM). Chimeric VLPs with inserts from Table 1 are shown to yield capsid-like structures ˜50 nm in diameter with a variegated appearance consistent with formation from smaller capsomere-like structures and resembling native HPV type 1 VLPs.

### Example 3: Conjugation of Antigens to Polyionic VLPs

For conjugation to the VLP, peptides are solubilized in distilled water at 5 mg/ml (or dissolved at 5 mg/ml in DMSO, if they are not soluble in water). Peptides at a preferred concentration of 2.5 mg/ml, but lower if less soluble in the buffer, are reduced with 10 mM Bond-breaker TCEP solution (Thermo Fisher Scientific) for 20 min at 50° C. After dialysis in 20 mM phosphate buffer, pH 6.5, 0.15 M NaCl, VLP protein (1 mg/ml), and peptide at peptide: L1 protein molar ratios between 4:1 to 16:1, based on solubility characteristics, are mixed in the presence of 4 mM glutathione disulfide (GSSG) and 0.8 mM reduced glutathione (GSH), and incubated overnight at 37° C. To remove unreacted peptide, reactants are dialyzed against 20 mM phosphate pH 6.5, 0.5 M NaCl, using dialysis tubing with a cut-off of 1 million kDa. The VLP-peptide solutions are adjusted to 0.01% Tween 80, 0.05% carboxymetyl cellulose, 0.5 mM GSSG and 0.05 mM GSH, aliquoted, and stored at −20° C. The amount of peptide bound to the VLP is determined by SDS-PAGE analysis and interpolation of sample-peptide band density from a standard curve of known amounts of peptide. Gels are scanned in a BioRad ChemiDocXR imager, and images are analyzed with NIH ImageJ software.

### Example 4: Ability of Exemplary Embodiments of VLPs with Various Inserts in the HI Loop to Induce a Cytolytic CD8+ T Cell Response

VLPs generated with various exemplary inserts in the L1 protein (Table 1) are linked via a TAG to a representative peptide encoding an MHC class I restricted epitope recognized in the genetic background of C57BL/6 mice. Mice are immunized by the intradermal route, as described below. The immune response is measured as described below. VLPs formulated with different inserts linked to a representative antigen are shown to induce comparable frequencies of antigen specific, interferon-γ secreting CD8+ T cells (no significant differences in mean responses by t-test comparison) cytolytic T cell responses.

### Example 5: Ability of VLPs Linked to an Antigen by Exemplary Embodiments of TAGs to Induce a Cytolytic CD8+ T Cell Response

A representative peptide encoding an MHC class I-restricted epitope recognized in the genetic background of C57BL/6 mice is chemically synthesized to >90% purity with the TAGs listed in Table 2. Peptides with TAGs-1 to -4, and -11 and -12 are linked to a polyionic VLP with Insert-1. Peptides with TAGs 5-10 are linked to VLPs with inserts 5-10, respectively. Mice are immunized by the intradermal route, as described below. The immune response is measured as described below. VLPs linked to a representative antigen formulated with different TAGs are shown to induce comparable frequencies of antigen specific, interferon-γ secreting CD8+ T cells (no significant differences in mean responses by t-test comparison).

### Example 6: Immunization with Polyionic VLPs and Detection of Antigen Specific CD8+ T Cell Responses

C57BL/6J mice 6-8 weeks of age are immunized three times, 1 week apart with between 5-50 ug each of VLP-peptide, administered by intradermal injection. As a control, C57BL/6J mice are immunized with unlabeled (no peptide) VLP protein. VLP-peptide immunogens are formulated with the set of extended peptides for the difference SARS-CoV-2 and OC43 antigens described in Table 3-12. For intradermal immunization, the VLP/antigen vaccine is injected in a single, or split doses, into the skin of the back of shaved mice.

To provide samples for immunological assays, mice are sacrificed 10-14 days after the last dose of vaccine and spleens are dissected. Splenocytes (or suspensions of CD3+ cells from lung tissue), are stimulated with 1 μg/ml of a pool of overlapping peptides 11 amino acids in length (overlapping by 8 amino acids) spanning the full-length amino acid sequence of the target antigen, in the presence of brefeldin A (10 ug/ml) overnight at 37° C. in 5% CO2. Cells are stained with Zombie Green™ fixable viability dye, treated with Fixation buffer and stored in Cytolast. Cells are permeabilized with Permeabilization buffer and stained with Brilliant Violet BV™ 510-conjugated anti-mouse CD3, clone 17A2, PerCPCy5.5-conjugated anti-mouse CD8α, clone 53-6.7, and PE-conjugated anti-mouse IFNγ, clone XMG1.2. Reagents are purchased from a commercial source. Flow cytometry is performed on an LSR-II or comparable flow cytometer and data are analyzed using FACSDiva software or FlowJo software. Gating is done on forward and side scatter parameters to select for lymphocytes and singlets. After exclusion of dead cells, CD8+T lymphocytes are identified on a CD3/CD8 dot plot of gated lymphocytes, and interferon-γ (IFNγ) secreting cells are identified on a CD8/IFNγ dot plot of gated CD8+ T cells. A minimum of 30,000 CD8+ T cells are analyzed. VLP-peptide immunogens of the M, N, S2, ORF3a, ORF7a, nsp6, nsp7, and nsp12 antigens of SARS-CoV-2, and immunogens of the M, N, S2, nsp6, nsp7, and nsp12 antigens of OC43 are shown to induce detectable antigen specific, interferon-γ secreting CD8+ T cells

### Example 7: Ability of Polyionic VLP SARS-CoV-2 Vaccines to Protect Mice Against SARS-CoV-2 Disease in a Challenge Model

Mice strain: Stable humanized angiotensin converting enzyme-II (ACE2) mice generated using CRISPR/Cas9 knock-in technology to replace the endogenous mouse ACE2 (mACE2) with the human ACE2 in the C57BL/6 strain of mouse will be used for SARS-CoV-2 challenge (Sun et al., 2020).

Mouse immunization: VLPs with an exemplary insert in the HI loop are formulated with the set of exemplary extended peptides with an exemplary and appropriate TAG for the particular insert. A polyionic VLP vaccine is formulated with M, N, S2, ORF3a, ORF7a, nsp6, nsp7 and/or nsp12 SARS-CoV-2 antigens of SARS-CoV-2 antigens. Peptides are drawn from the list in Tables 3-10 with SEQ ID ending in extension, -E1, -E2, -E3, etc. The precise number of peptides will depend on the antigen as described in the table. The preferred insert is the E8C amino acid sequence at amino acid positions 347 to 355 in the HI loop of the L1 protein of bovine papillomavirus type 1, with replacement of native amino acids (insert-1, Table 1), and the preferred TAG is CRRRRRRRRCAAYY (TAG-1, in Table 2). Additional insert and TAG combinations with sets of peptides for one or more SARS-CoV-2 antigens are also tested, as informed by other enabling experiments. VLP/antigen constructs are generated as described above. Mice are immunized by the intradermal route, intranasal/lung route, or both routes simultaneously. For intradermal immunization, the VLP/antigen vaccine is injected in a single, or split doses, into the skin of the back of shaved mice. For nasal/pulmonary immunization, the VLP/antigen construct is administered dropwise (10 μl) into the nose of a lightly anesthetized mouse. In anesthetized mice intra-nasally administered vaccine is also inhaled by the mouse and thus is delivered to both the lung and nasopharyngeal tissues.

Mouse challenge: For intranasal infection, aged (30 weeks old) hACE2 mice are anesthetized with Isoflurane delivered with a precision vaporizer, and then intranasally infected with 4×105 pfu of SARSCoV-2. Mice are then weighed and monitored daily and sacrificed on day 6 post infection for serum collection and tissue processing. Spleens and lung tissue are collected for analysis of viral RNA load, histopathology, and measurement of cellular immune response.

Measurement of viral RNA load: Viral RNA in lung tissue is extracted with a RNeasy Mini kit (QIAGEN) according to the protocols. The viral RNA quantification is performed by RT-qPCR targeting the S gene of SARS-CoV-2. RT-qPCR is performed using One Step PrimeScript RT-PCR Kit (Takara) with the following primers and probes: CoV-F3, CoV-R3 and CoV-P3 (Sun et al., 2020).

Antigen specific CD8+ T cell responses: CD8+ T cell response of splenocytes and CD3+ T cells recovered from lung homogenates are measured as described above.

SARS-CoV-2 polyionic VLP vaccines are shown to induce antigen specific CD8+ T cells responses detectable in splenocytes and in CD3+ T cells from lung tissue, to each of the target antigen (M, N S2, ORF3a and ORF7a). Vaccinated mice are further shown to have a significantly reduced viral RNA load and less lung pathology than mock vaccinated mice. Vaccines are also shown to decrease expression of viral RNA in the lung. Of note, the vaccines are not expected to provide sterilizing immunity.

### Example 8: Ability of Polyionic VLP SARS-CoV-2 Vaccines to Protect Syrian Hamsters Against SARS-CoV-2 Infection in a Challenge Model

Animal species: The Syrian hamster is highly susceptible to SARS-CoV-2 infection, making it the most suitable small animal model to evaluate the protective efficacy of vaccines (Rosenke et al., 2020; Chan et al. 2020). Syrian hamsters (Mesocricetus auratus), 6-8 weeks of age are purchased from Jackson Laboratories.

Hamster immunization: VLPs with an exemplary insert in the HI loop are formulated with the set of exemplary extended peptides with an exemplary and appropriate TAG for the particular insert. A polyionic VLP vaccine is formulated with M, N, S2, ORF3a, ORF7a, nsp6, nsp7 and/or nsp12 SARS-CoV-2 antigens. Peptides are drawn from the list in Tables 3-10 with SEQ ID ending in extension, -E1, -E2, -E3, etc. The precise number of peptides will depend on the antigen as described in the table and may include all or fewer antigens with a-E extension designation. The preferred insert is the E8C amino acid sequence at amino acid positions 347 to 355 in the HI loop of the L1 protein of bovine papillomavirus type 1, with replacement of native amino acids (insert-1, Table 1), and the preferred TAG is CRRRRRRRRCAAYY (TAG-1, in Table 2). Additional insert and TAG combinations with sets of peptides for one or more SARS-CoV-2 antigens are also tested, as informed by other enabling experiments. VLP/antigen constructs are generated as described above. Hamsters are immunized by the intradermal route, intranasal route, or both routes individually or in combination. Intradermal immunization is performed as described above. For nasal immunization, the VLP/antigen construct is administered dropwise (10 μl) into the nose of a lightly anesthetized mouse.

Hamster challenge: To mimic the natural route of infection, vaccinated animals and controls (contacts) will be exposed to previously infected animals (index) by co-housing in the same cage. For intranasal infection of the index animal, hamsters are anesthetized with Isoflurane delivered with a precision vaporizer, and then intranasally infected with 100 tissue culture dose 50 (TCID₅₀) of SARSCoV-2 virus. SARS-CoV-2 isolate nCOV-WA1-2020 (MN985325.1) from the CDC, or a suitable alternative isolate, is obtained and propagated in Vero E6 cells. The TCID₅₀ dose is determined by titration of the viral stock in VeroE6 cells. The infected hamster and co-housed vaccinated and control naïve hamsters are weighed and monitored daily for clinical signs of disease. To monitor infection by RT-qPCR, nasal washes are collected from lightly anesthetized naïve contact (vaccinated and control) and index (previously infected) animals daily for 10 days by instillation and then collection of 150 μl of PBS/0.3% BSA in both nostrils.

Measurement of viral RNA load: Viral RNA in lung tissue is extracted with a RNeasy Mini kit (QIAGEN) according to the protocols. The viral RNA quantification is performed by RT-qPCR targeting the S gene of SARS-CoV-2. RT-qPCR is performed using One Step PrimeScript RT-PCR Kit (Takara) with the following primers and probes: CoV-F3, CoV-R3 and CoV-P3 (Sun et al., 2020).

SARS-CoV-2 polyionic VLP vaccinated hamsters are shown to have a significantly reduced viral RNA load in nasal washes than mock vaccinated mice after exposure to an infected index hamster.

### Tables

TABLE 1
Representative negatively charged amino acid-
cysteine sequences in HI loop

| | | Replaced | Position(s) of |
|---|---|---|---|
| | Inserted aa | native aa | replacement in |
| Seq ID | sequence | sequence | BPV1 L1 ORF |
| Insert-1 | EEEEEEEEC | GTPLTEYDS | Aa347-355 |
| Insert-2 | CEEEEEEE | GTPLTEYDS | Aa347-355 |
| Insert-3 | DDDDDDDDC | GTPLTEYDS | Aa347-355 |
| Insert-4 | EDEDEDEDC | GTPLTEYDS | Aa347-355 |
| Insert-5 | EEEEC | GTPLT | Aa347-351 |
| Insert-6 | EEEEEC | GTPLTE | Aa347-352 |
| Insert-7 | EEEEEEC | GTPLTEY | aa347-353 |
| Insert-8 | EEEEEEEC | GTPLTEYD | Aa347-355 |
| Insert-9 | EEEEEEEEEC | DGTPLTEYDS | Aa346-355 |
| Insert-10 | EEEEEEEEEEC | DGTPLTEYDSS | Aa346-356 |
| Insert-11 | CEEEEEEEEC | DGTPLTEYDS | aa346-355 |
| Insert-12 | EEEEEEEEC | TPLTEYD | aa348-354 |
| Insert-13 | EEEEEEEEC | TPLTE | aa348-352 |
| Insert-14 | EEEEEEEEC | TPL | aa347-350 |
| Insert-15 | EEEEEEEEC | DGTPLTEYDS | aa346-3556 |
| Insert-16 | EEEEEEEEC | P | aa349 |
| Insert-17 | GSSGEEEEEEE | P | aa349 |
| | ECGSSG | | |
| Insert-18 | EEEEEEEEC | None | Between |
| | | | aa349-350 |
| Insert-19 | GSSGEEEEEEE | none | Between |
| | ECGSSG | | aa349-350 |

TABLE 2
Representative TAG sequences

| | | Amino acid |
|---|---|---|
| Seq ID | TAG designation | sequence¹ |
| TAG-1 | polyR8 | RRRRRRRRC |
| TAG-2 | PolyK8 | KKKKKKKKC |
| TAG-3 | PolyH8 | HHHHHHHHC |
| TAG-4 | MixedRKH8 | RKHRKHRKC |
| TAG-5 | PolyR4 | RRRRC |
| TAG-6 | PolyR5 | RRRRRC |
| TAG-7 | PolyR6 | RRRRRRC |
| TAG-8 | PolyR7 | RRRRRRRC |
| TAG-9 | PolyR9 | RRRRRRRRRC |
| TAG-10 | PolyR10 | RRRRRRRRRRC |
| TAG-11 | NH-terminal C | CRRRRRRRR |
| TAG-12 | Dual C | CRRRRRRRRC |

¹All TAG amino acid sequences include a C-terminal AAYY proteolytic processing sequence.

TABLE 3
SARS-CoV-2 antigens of the M structural protein.

| Immunogen | Seq ID | aa-position¹ | Amino acid sequence² |
|---|---|---|---|
| Short | SARS-CoV-2-M-S1 | aa6-36 | GTITVEELKKLLEQWNLVIGFLFLTWIC |
| peptide | | | LLQ |
| | SARS-CoV-2-M-S2 | aa27-56 | LFLTWICLLQFAYANRNRFLYIIKLIFLY |
| | | | WL |
| | SARS-CoV-2-M-S3 | aa48-77 | IIKLIFLWLLWPVTLACFVLAAVYRIN |
| | | | WIT |
| | SARS-CoV-2-M-S4 | aa68-97 | AAVYRINWITGGIAIAMACLVGLMWL |
| | | | SYFI |
| | SARS-CoV-2-M-S5 | aa88-117 | VGLMWLSYFIASFRLFARTRSMWSFNP |
| | | | ETN |
| | SARS-CoV-2-M-S6 | aa108-138 | SMWSFNPETNILLNVPLHGTILTRPLLE |
| | | | SEL |
| | SARS-CoV-2-M-S7 | aa129-159 | LTRPLLESELVIGAVILRGHLRIAGHHL |
| | | | GRC |
| | SARS-CoV-2-M-S8 | aa150-179 | RIAGHHLGRCDIKDLPKEITVATSRTLS |
| | | | YY |
| | SARS-CoV-2-M-S9 | aa170-200 | VATSRTLSYYKLGASQRVAGDSGFAA |
| | | | YSRYR |
| | SARS-CoV-2-M-S10 | aa191-221 | SGFAAYSRYRIGNYKLNTDHSSSSDNI |
| | | | ALLV |
| Extended | SARS-CoV2-M-E1 | aa6-50 | GTITVEELKKLLEQWNLVIGFLFLTWIC |
| peptide | | | LLQFAYANRNRFLYIIK |
| | SARS-CoV2-M-E2 | aa43-85 | NRNRFLYIIKLIFLWLLWPVTLACFVL |
| | | | AAVYRINWITGGIAIAMA |
| | SARS-CoV2-M-E3 | aa76-120 | ITGGIAIAMACLVGLMWLSYFIASFRLF |
| | | | ARTRSMWSFNPETNILL |
| | SARS-CoV2-M-E4 | aa111-155 | SFNPETNILLNVPLHGTILTRPLLESELV |
| | | | IGAVILRGHLRIAGHH |
| | SARS-CoV2-M-E5 | aa146-190 | RGHLRIAGHHLGRCDIKDLPKEITVAT |
| | | | SRTLSYYKLGASQRVAGD |
| | SARS-CoV2-M-E6 | aa181-221 | LGASQRVAGDSGFAAYSRYRIGNYKL |
| | | | NTDHSSSSDNIALLV |
| Short protein | SARS-CoV2-M-P1 | aa6-116 | GTITVEELKKLLEQWNLVIGFLFLTWIC |
| | | | LLQFAYANRNRFLYIIKLIFLWLLWPV |
| | | | TLACFVLAAVYRINWITGGIAIAMACL |
| | | | VGLMWLSYFIASFRLFARTRSMWSFNP |
| | | | ET |
| | SARS-CoV2-M-P2 | aa106-221 | TRSMWSFNPETNILLNVPLHGTILTRP |
| | | | LLESELVIGAVIL |
| | | | RGHLRIAGHHLGRCDIKDLPKEITVAT |
| | | | SRTLSYYKLGASQRVAGDSGFAAYSRY |
| | | | RIGNYKLNTDHSSSSDNIALLV |
| Full length | SARS-CoV-2-FL | aa6-221 | GTITVEELKKLLEQWNLVIGFLFLTWIC |
| target | | | LLQFAYANRNRFLYIIKLIFLWLLWPV |
| | | | TLACFVLAAVYRINWITGGIAIAMACL |
| | | | VGLMWLSYFIASFRLFARTRSMWSFNP |
| | | | ETNILLNVPLHGTILTRPLLESELVIGA |
| | | | VILRGHLRIAGHHLGRCDIKDLPKEIT |
| | | | VATSRTLSYYKLGASQRVAGDSGFAA |
| | | | YSRYRIGNYKLNTDHSSSSDNIALLV |

¹aa positions based on SARS-CoV-2 reference genome (NC-06577.2 (Wuhan strain) and reference M protein sequences, YP_009724393.1.
²For conjugation to polyionic VLPS, the peptide/ protein antigens have a N-terminal TAG and AAYY proteolytic processing sequence.

TABLE 4
SARS-CoV-2 antigens of the N structural protein

| Immunogen | Seq ID | aa-position¹ | Amino acid sequence² |
|---|---|---|---|
| Short | SARS-CoV-2-N- | aa51-84 | SWFTALTQHGKEDLKFPRGQGVPINTNSSP |
| peptide | S1 | | DDQI |
| | SARS-CoV-2-N- | aa75-108 | NTNSSPDDQIGYYRRATRRIRGGDGKMKD |
| | S2 | | LSPRW |
| | SARS-CoV-2-N- | aa99-132 | GKMKDLSPRWYFYYLGTGPEAGLPYGAN |
| | S3 | | KDGIIW |
| | SARS-CoV-2-N- | aa123-156 | YGANKDGIIWVATEGALNTPKDHIGTRNP |
| | S4 | | ANNAA |
| | SARS-CoV-2-N- | aa147-180 | GTRNPANNAAIVLQLPQGTTLPKGFYAEG |
| | S5 | | SRGGS |
| | SARS-CoV-2-N- | aa171-204 | FYAEGSRGGSQASSRSSSRSRNSSRNSTPGSS |
| | S6 | | RG |
| | SARS-CoV-2-N- | aa195-227 | RNSTPGSSRGTSPARMAGNGGDAALALLL |
| | S7 | | LDRLN |
| | SARS-CoV-2-N- | aa219-252 | LALLLLDRLNQLESKMSGKGQQQQGQTV |
| | S8 | | TKKSAA |
| | SARS-CoV-2-N- | aa243-276 | GQTVTKKSAAEASKKPRQKRTATKAYNVT |
| | S9 | | QAFGR |
| | SARS-CoV-2-N- | aa267-300 | AYNVTQAFGRRGPEQTQGNFGDQELIRQG |
| | S10 | | TDYKH |
| | SARS-CoV-2-N- | aa291-324 | LIRQGTDYKHWPQIAQFAPSASAFFGMSRI |
| | S11 | | GMEV |
| | SARS-CoV-2-N- | aa315-347 | FGMSRIGMEVTPSGTWLTYTGAIKLDDKD |
| | S12 | | PNFK |
| | SARS-CoV-2-N- | aa338-369 | KLDDKDPNFKDQVILLNKHIDAYKTFPPTE |
| | S13 | | PK |
| Extended | SARS-CoV2-N- | aa51-104 | SWFTALTQHGKEDLKFPRGQGVPINTNSSP |
| peptide | E1 | | DDQIGYYRRATRRIRGGDGKMKDL |
| | SARS-CoV2-N- | aa95-148 | RGGDGKMKDLSPRWYFYYLGTGPEAGLPY |
| | E2 | | GANKDGIIWVATEGALNTPKDHIGT |
| | SARS-CoV2-N- | aa139-192 | LNTPKDHIGTRNPANNAAIVLQLPQGTTL |
| | E3 | | PKGFYAEGSRGGSQASSRSSSRSRN |
| | SARS-CoV2-N- | aa183-235 | SSRSSSRSRNSSRNSTPGSSRGTSPARMAGN |
| | E4 | | GGDAALALLLLDRLNQLESKMS |
| | SARS-CoV2-N- | aa227-280 | LNQLESKMSGKGQQQQGQTVTKKSAAEA |
| | E5 | | SKKPRQKRTATKAYNVTQAFGRRGPE |
| | SARS-CoV2-N- | aa271-325 | TQAFGRRGPEQTQGNFGDQELIRQGTDYK |
| | E6 | | HWPQIAQFAPSASAFFGMSRIGMEVT |
| | SARS-CoV2-N- | aa316-369 | GMSRIGMEVTPSGTWLTYTGAIKLDDKDP |
| | E7 | | NFKDQVILLNKHIDAYKTFPPTEPK |
| Short | SARS-CoV2-N- | aa51-162 | SWFTALTQHGKEDLKFPRGQGVPINTNSSP |
| protein | P1 | | DDQIGYYRRATRRIRGGDGKMKDLSPRWY |
| | | | FYYLGTGPEAGLPYGANKDGIIWVATEGA |
| | | | LNTPKDHIGTRNPANNAAIVLQLPG |
| | SARS-CoV2-N- | aa153-265 | NNAAIVLQLPQGTTLPKGFYAEGSRGGSQ |
| | P2 | | ASSRSSSRSRNSSRNSTPGSSRGTSPARMAG |
| | | | NGGDAALALLLLDRLNQLESKMSGKGQQ |
| | | | QQGQTVTKKSAAEASKKPRQKRTAT |
| | SARS-CoV2-N- | aa255-369 | SKKPRQKRTATKAYNVTQAFGRRGPEQTQ |
| | P3 | | GNFGDQELIRQGTDYKHWPQIAQFAPSAS |
| | | | AFFGMSRIGMEVTPSGTWLTYTGAIKLDDK |
| | | | DPNFKDQVILLNKHIDAYKTFPPTEPK |
| Full length | SARS-CoV-2-N- | aa51-369 | SWFTALTQHGKEDLKFPRGQGVPINTNSSP |
| | FL | | DDQIGYYRRATRRIRGGDGKMKDLSPRWY |
| | (319 aa) | | FYYLGTGPEAGLPYGANKDGIIWVATEGA |
| | | | LNTPKDHIGTRNPANNAAIVLQLPQGTTL |
| | | | PKGFYAEGSRGGSQASSRSSSRSRNSSRNST |
| | | | PGSSRGTSPARMAGNGGDAALALLLLDRL |
| | | | NQLESKMSGKGQQQQGQTVTKKSAAEAS |
| | | | KKPRQKRTATKAYNVTQAFGRRGPEQTQ |
| | | | GNFGDQELIRQGTDYKHWPQIAQFAPSAS |
| | | | AFFGMSRIGMEVTPSGTWLTYTGAIKLDDK |
| | | | DPNFKDQVILLNKHIDAYKTFPPTEPK |

¹aa positions based on SARS-CoV-2 reference genome (NC-06577.2 (Wuhan strain) and reference N protein sequences, YP_009724397.2.
²For conjugation to polyionic VLPS, the peptide/protein antigens have a N-terminal TAG and AAYY proteolytic processing sequence.

TABLE 5
SARS-CoV-2 antigens of the S2 structural protein.

| Immunogen | Seq ID | aa-position¹ | Amino acid sequence² |
|---|---|---|---|
| Extended | SARS-CoV2- | aa671-725 | CASYQTQTNSPRRARSVASQSIIAYTMSLGA |
| peptide | S2-E1 | | ENSVAY |
| | | | SNNSIAIPTNFTISVTTE |
| | SARS-CoV2- | aa716-769 | TNFTISVTTEILPVSMTKTSVDCTMYICGDST |
| | S2-E2 | | ECSNLL |
| | | | LQYGSFCTQLNRALTG |
| | SARS-CoV2- | aa760-811 | CTQLNRALTGIAVEQDKNTQEVFAQVKQIY |
| | S2-E3 | | KTPPIKDF |
| | | | GGFNFSQILPDPSKPSK |
| | SARS-CoV2- | aa805-860 | ILPDPSKPSKRSFIEDLLFNKVTLADAGFIKQ |
| | S2-E4 | | YGDCLGDIA |
| | | | ARDLICAQKFNGLTV |
| | SARS-CoV2- | aa851-905 | CAQKFNGLTVLPPLLTDEMIAQYTSALLAG |
| | S2-E5 | | TITSGWTF |
| | | | GAGAALQIPFAMQMAYR |
| | SARS-CoV2- | aa897-951 | PFAMQMAYRFNGIGVTQNVLYENQKLIAN |
| | S2-E6 | | QFNSAIG |
| | | | KIQDSLSSTASALGKLQDV |
| | SARS-CoV2- | aa942-996 | ASALGKLQDVVNQNAQALNTLVKQLSSNF |
| | S2-E7 | | GAISSVLN |
| | | | DILSRLDKVEAEVQIDRL |
| | SARS-CoV2- | aa987-1041 | VEAEVQIDRLITGRLQSLQTYVTQQLIRAAEI |
| | S2-E8 | | RASANLA |
| | | | ATKMSECVLGQSKRVD |
| | SARS-CoV2- | aa1032-1087 | CVLGQSKRVDFCGKGYHLMSFPQSAPHGV |
| | S2-E9 | | VFLHVTYV |
| | | | PAQEKNFTTAPAICHDGKA |
| | SARS-CoV2- | aa1078-1132 | APAICHDGKAHFPREGVFVSNGTHWFVTQ |
| | S2-E10 | | RNFYEP |
| | | | QIITTDNTFVSGNCDVVIGI |
| | SARS-CoV2- | aa1123-1177 | SGNCDVVIGIVNNTVYDPLQPELDSFKEELD |
| | S2-E11 | | KYFKNHT |
| | | | SPDVDLGDISGINASVV |
| | SARS-CoV2- | aa1168-1222 | DISGINASVVNIQKEIDRLNEVAKNLNESLI |
| | S2-E12 | | DLQELGKYE |
| | | | QYIKWPWYIWLGFIA |
| | SARS-CoV2- | aa1213-1268 | PWYIWLGFIAGLIAIVMVTIMLCCMTSCCSC |
| | S2-E13 | | LKGCCSCGS |
| | | | CCKFDEDDSEPVLKGV |
| Short protein | SARS-CoV2- | aa671-798 | CASYQTQTNSPRRARSVASQSIIAYTMSLGA |
| | S2-P1 | | ENSVAYSNN |
| | | | SIAIPTNFTISVTTEILPVSMTKTSVDCTMYIC |
| | | | GDSTECSNLL |
| | | | LQYGSFCTQLNRALTGIAVEQDKNTQEVFA |
| | | | QVKQIYKTPPIKDFG |
| | SARS-CoV2- | aa788-916 | IYKTPPIKDFGGFNFSQILPDPSKPSKRSFIED |
| | S2-P2 | | LLFNKVTLADA |
| | | | GFIKQYGDCLGDIAARDLICAQKFNGLTVL |
| | | | PPLLTDEMIAQYTSALLAGTITSGWTFGAG |
| | | | AALQIPFAMQMAYRFNGIGVTQNVL |
| | SARS-CoV2- | aa906-1033 | FNGIGVTQNVLYENQKLIANQFNSAIGKIQ |
| | S2-P3 | | DSLSSTASALGK |
| | | | LQDVVNQNAQALNTLVKQLSSNFGAISSVL |
| | | | NDILSRLDKVE |
| | | | AEVQIDRLITGRLQSLQTYVTQQLIRAAEIR |
| | | | ASANLAATKMSECV |
| | SARS-CoV2- | aa1023-1152 | NLAATKMSECVLGQSKRVDFCGKGYHLMS |
| | S2-P4 | | FPQAPHGVV |
| | | | FLHVTYVPAQEKNFTTAPAICHDGKAHFPR |
| | | | EGVFVSNG |
| | | | THWFVTQRNFYEPQIITTDNTFVSGNCDVVI |
| | | | GIVNNTVYD |
| | | | PLQPELIDSFKEEL |
| | SARS-CoV2- | aa1142-1268 | QPELIDSFKEELDKYFKNHTSPDVDLGDISGI |
| | S2-P5 | | NASVVNIQKE |
| | | | DRLNEVAKNLNESLIDLQELGKYEQYIKWP |
| | | | WYIWLGFIAG |
| | | | LIAIVMVTIMLCCMTSCCSCLKGCCSCGSCC |
| | | | KFDEDDSEPVLKGV |

^{l}aa positions based on SARS-CoV-2 reference genome (NC-06577.2 (Wuhan strain) and reference spike protein sequences, YP_009724390.1.
²For conjugation to polyionic VLPS, the peptide/protein antigens have a N-terminal TAG and AAYY proteolytic processing sequence.

TABLE 6
SARS-CoV-2 antigens
of the ORF3a structural protein

| | | aa- | |
|---|---|---|---|
| Immunogen | Seq ID | position¹ | Amino acid sequence² |
| Short | SARS- | aa105-132 | FLYLYALVYFLQSINFVRII |
| peptide | CoV2- | | MRLWLCWK |
| | ORF3a- | | |
| | S1 | | |
| | SARS- | aa123-147 | IIMRLWLCWKCRSKNPLLYD |
| | CoV2- | | ANYFL |
| | ORF3a- | | |
| | S2 | | |
| | SARS- | aa138-165 | PLLYDANYFLCWHTNCYDYC |
| | CoV2- | | IPYNSVTS |
| | ORF3a- | | |
| | S3 | | |
| | SARS- | aa156-183 | YCIPYNSVTSSIVITSGDGT |
| | CoV2- | | TSPISEHD |
| | ORF3a- | | |
| | S4 | | |
| | SARS- | aa174-201 | GTTSPISEHDYQIGGYTEKW |
| | CoV2- | | ESGVKDCV |
| | ORF3a- | | |
| | S5 | | |
| | SARS- | aa192-219 | KWESGVKDCVVLHSYFTSDY |
| | CoV2- | | YQLYSTQL |
| | ORF3a- | | |
| | S6 | | |
| | SARS- | aa210-238 | DYYQLYSTQLSTDTGVEHVT |
| | CoV2- | | FFIYNKIVD |
| | ORF3a- | | |
| | S7 | | |
| | SARS- | aa229-257 | TFFIYNKIVDEPEEHVQIHT |
| | CoV2- | | IDGSSGVVN |
| | ORF3a- | | |
| | S8 | | |
| | SARS- | aa248-275 | TIDGSSGVVNPVMEPIYDEP |
| | CoV2- | | TTTTSVPL |
| | ORF3a- | | |
| | S9 | | |
| Extended | SARS- | aa105-154 | FLYLYALVYFLQSINFVRII |
| peptide | CoV2- | | MRLWLCWKCRSKNPLLYDAN |
| | ORF3a- | | YFLCWHTNCY |
| | E1 | | |
| | SARS- | aa145-194 | YFLCWHTNCYDYCIPYNSVT |
| | CoV2- | | SSIVITSGDGTTSPISEHDY |
| | ORF3a- | | QIGGYTEKWE |
| | E2 | | |
| | SARS- | aa185-234 | QIGGYTEKWESGVKDCVVLH |
| | CoV2- | | SYFTSDYYQLYSTQLSTDTG |
| | ORF3a- | | VEHVTFFIYN |
| | E3 | | |
| | SARS- | aa225-275 | VEHVTFFIYNKIVDEPEEHV |
| | CoV2- | | QIHTIDGSSGVVNPVMEPIY |
| | ORF3a- | | DEPTTTTSVPL |
| | E4 | | |
| Short | SARS- | aa105-195 | FLYLYALVYFLQSINFVRII |
| protein | CoV2- | | MRLWLCWKCRSKNPLLYDAN |
| | ORF3a- | | YFLCWHTNCYDYCIPYNSVT |
| | P1 | | SSIVITSGDGTTSPISEHDY |
| | | | QIGGYTEKWES |
| | SARS- | aa185-275 | QIGGYTEKWESGVKDCVVLH |
| | CoV2- | | SYFTSDYYQLYSTQLSTDTG |
| | ORF3a- | | VEHVTFFIYNKIVDEPEEHV |
| | P2 | | QIHTIDGSSGVVNPVMEPIY |
| | | | DEPTTTTSVPL |
| Full- | SAR- | aa105-275 | FLYLYALVYFLQSINFVRII |
| length | CoV2- | | MRLWLCWKCRSKNPLLYDAN |
| | ORF3a- | | YFLCWHTNCYDYCIPYNSVT |
| | FL | | SSIVITSGDGTTSPISEHDY |
| | (171 | | QIGGYTEKWESGVKDCVVLH |
| | aa) | | SYFTSDYYQLYSTQLSTDTG |
| | | | VEHVTFFIYNKIVDEPEEHV |
| | | | QIHTIDGSSGVVNPVMEPIY |
| | | | DEPTTTTSVPL |

¹aa positions based on SARS-CoV-2 reference genome (NC-06577.2 (Wuhan strain) and reference ORF3a protein sequences, YP_009724391.1.
²For conjugation to polyionic VLPS, the peptide/protein antigens have a N-terminal TAG and AAYY proteolytic processing sequence.

TABLE 7
SARS-CoV-2 antigens
of the ORF7a structural protein

| | | aa- | |
|---|---|---|---|
| Immunogen | Seq ID | position¹ | Amino acid sequence² |
| Short | SARS- | aa1-28 | MKIILFLALITLATCELYHY |
| peptide | CoV2- | | QECVRGTT |
| | ORF7a- | | |
| | S1 | | |
| | SARS- | aa20-47 | YQECVRGTTVLLKEPCSSGT |
| | CoV2- | | YEGNSPFH |
| | ORF7a- | | |
| | S2 | | |
| | SARS- | aa38-65 | GTYEGNSPFHPLADNKFALT |
| | CoV2- | | CFSTQFAF |
| | ORF7a- | | |
| | S3 | | |
| | SARS- | aa56-84 | LTCFSTQFAFACPDGVKHVY |
| | CoV2- | | QLRARSVSP |
| | ORF7a- | | |
| | S4 | | |
| | SARS- | aa75-103 | YQLRARSVSPKLFIRQEEVQ |
| | CoV2- | | ELYSPIFLI |
| | ORF7a- | | |
| | S5 | | |
| | SARS- | aa94-121 | QELYSPIFLIVAAIVFITLC |
| | CoV2- | | FTLKRKTE |
| | ORF7a- | | |
| | S6 | | |
| Extended | SARS- | aa1-46 | MKIILFLALITLeATCELYH |
| peptide | CoV-2- | | YQECVRGTTVLLKEPCSSGT |
| | ORF7a- | | YEGNSPFH |
| | E1 | | |
| | SARS- | aa38-84 | GTYEGNSPFHPLADNKFALT |
| | CoV-2- | | CFSTQFAFACPDGVKHVYQL |
| | ORF7a- | | RARSVSP |
| | E2 | | |
| | SARS- | aa75-121 | YQLRARSVSPKLFIRQEEVQ |
| | CoV-2- | | ELYSPIFLIVAAIVFITLCF |
| | ORF7a- | | TLKRKTE |
| | E3 | | |
| Full | SARS- | aa1-121 | MKIILFLALITLATCELYHY |
| length | CoV2- | | QECVRGTTVLLKEPCSSGTY |
| | ORF7a- | | EGNSPFHPLADNKFALTCFS |
| | FL | | TQFAFACPDGVKHVYQLRAR |
| | | | SVSPKLFIRQEEVQELYSPI |
| | | | FLIVAAIVFITLCFTLKRKT |
| | | | E |

¹aa positions based on SARS-CoV-2 reference genome (NC-06577.2 (Wuhan strain) and reference ORF7a protein sequences, YP_009724395.1
²For conjugation to polyionic VLPS, the peptide/protein antigens have a N-terminal TAG and AAYY proteolytic processing sequence.

TABLE 8
SARS-CoV-2 antigens
of nsp6 and nsp7 nonstructural proteins

| | | aa- | |
|---|---|---|---|
| Immunogen | Seq ID | position¹ | Amino acid sequence² |
| Short | SARS- | 1-30 | SAVKRTIKGTHHWLLLTILT |
| peptide | CoV-2 | | SLLVLVQSTQ |
| | nsp6-S1 | | |
| | SARS- | 21-50 | SLLVLVQSTQWSLFFFLYEN |
| | CoV-2 | | AFLPFAMGII |
| | nsp6-S2 | | |
| | SARS- | 41-70 | AFLPFAMGIIAMSAFAMMFV |
| | CoV-2 | | KHKHAFLCLF |
| | nsp6-S3 | | |
| | SARS- | 61-90 | KHKHAFLCLFLLPSLATVAY |
| | CoV-2 | | FNMVYMPASW |
| | nsp6-S4 | | |
| | SARS- | 81-110 | FNMVYMPASWVMRIMTWLDM |
| | CoV-2 | | VDTSLSGFKL |
| | nsp6-S5 | | |
| | SARS- | 101-130 | VDTSLSGFKLKDCVMYASAV |
| | CoV-2 | | VLLILMTART |
| | nsp6-S6 | | |
| | SARS- | 121-150 | VLLILMTARTVYDDGARRVW |
| | CoV-2 | | TLMNVLTLVY |
| | nsp6-S7 | | |
| | SARS- | 141-170 | TLMNVLTLVYKVYYGNALDQ |
| | CoV-2 | | AISMWALIIS |
| | nsp6-S8 | | |
| | SARS- | 161-190 | AISMWALIISVTSNYSGVVT |
| | CoV-2 | | TVMFLARGIV |
| | nsp6-S9 | | |
| | SARS- | 181-210 | TVMFLARGIVFMCVEYCPIF |
| | CoV-2 | | FITGNTLQCI |
| | nsp6-S10 | | |
| | SARS- | 201-230 | FITGNTLQCIMLVYCFLGYF |
| | CoV-2 | | CTCYFGLFCL |
| | nsp6-S11 | | |
| | SARS- | 221-250 | CTCYFGLFCLLNRYFRLTLG |
| | CoV-2 | | VYDYLVSTQE |
| | nsp6-S12 | | |
| | SARS- | 241-270 | VYDYLVSTQEFRYMNSQGLL |
| | CoV-2 | | PPKNSIDAFK |
| | nsp6-S13 | | |
| | SARS- | 261-290 | PPKNSIDAFKLNIKLLGVGG |
| | CoV-2 | | KPCIKVATVQ |
| | nsp6-S14 | | |
| | SARS- | 1-28 | SKMSDVKCTSVVLLSVLQQL |
| | CoV-2 | | RVESSSKL |
| | nsp7-S1 | | |
| | SARS- | 19-46 | QLRVESSSKLWAQCVQLHND |
| | CoV-2 | | ILLAKDTT |
| | nsp7-S2 | | |
| | SARS- | 37-64 | NDILLAKDTTEAFEKMVSLL |
| | CoV-2 | | SVLLSMQG |
| | nsp7-S3 | | |
| | SARS- | 55-83 | LLSVLLSMQGAVDINKLCEE |
| | CoV-2 | | MLDNRATLQ |
| | nsp7-S4 | | |
| Extended | SARS- | 1-50 | SAVKRTIKGTHHWLLLTILT |
| peptide | CoV-2 | | SLLVLVQSTQWSLFFFLYEN |
| | nsp6-E1 | | AFLPFAMGII |
| | SARS- | 41-90 | AFLPFAMGIIAMSAFAMMFV |
| | CoV-2 | | KHKHAFLCLFLLPSLATVAY |
| | nsp6-E2 | | FNMVYMPASW |
| | SARS- | 81-130 | FNMVYMPASWVMRIMTWLDM |
| | CoV-2 | | VDTSLSGFKLKDCVMYASAV |
| | nsp6-E3 | | VLLILMTART |
| | SARS- | 121-170 | VLLILMTARTVYDDGARRVW |
| | CoV-2 | | TLMNVLTLVYKVYYGNALDQ |
| | nsp6-E4 | | AISMWALIIS |
| | SARS- | 161-210 | AISMWALIISVTSNYSGVVT |
| | CoV-2 | | TVMFLARGIVFMCVEYCPIF |
| | nsp6-E5 | | FITGNTLQCI |
| | SARS- | 201-250 | FITGNTLQCIMLVYCFLGYF |
| | CoV-2 | | CTCYFGLFCLLNRYFRLTLG |
| | nsp6-E6 | | VYDYLVSTQE |
| | SARS- | 241-290 | VYDYLVSTQEFRYMNSQGLL |
| | CoV-2 | | PPKNSIDAFKLNIKLLGVGG |
| | nsp6-E7 | | KPCIKVATVQ |
| | SARS- | 1-46 | SKMSDVKCTSVVLLSVLQQL |
| | CoV-2 | | RVESSSKLWAQCVQLHNDIL |
| | nsp7-E1 | | LAKDTT |
| | SARS- | 37-83 | NDILLAKDTTEAFEKMVSLL |
| | CoV-2 | | SVLLSMQGAVDINKLCEEML |
| | nsp7-E2 | | DNRATLQ |
| Short | SARS- | 1-104 | SAVKRTIKGTHHWLLLTILT |
| protein | CoV-2 | | SLLVLVQSTQWSLFFFLYEN |
| | nsp6-SP1 | | AFLPFAMGIIAMSAFAMMFV |
| | | | KHKHAFLCLFLLPSLATVAY |
| | | | FNMVYMPASWVMRIMTWLDM |
| | | | VDTS |
| | SARS- | 94-197 | IMTWLDMVDTSLSGFKLKDC |
| | CoV-2 | | VMYASAVVLLILMTARTVYD |
| | nsp6-SP2 | | DGARRVWTLMNVLTLVYKVY |
| | | | YGNALDQAISMWALIISVTS |
| | | | NYSGVVTTVMFLARGIVFMC |
| | | | VEYC |
| | SARS- | 187-290 | RGIVFMCVEYCPIFFITGNT |
| | CoV-2 | | LQCIMLVYCFLGYFCTCYFG |
| | nsp6-SP3 | | LFCLLNRYFRLTLGVYDYLV |
| | | | STQEFRYMNSQGLLPPKNSI |
| | | | DAFKLNIKLLGVGGKPCIKV |
| | | | ATVQ |
| Full | SARS- | 1-290 | SAVKRTIKGTHHWLLLTILT |
| length | CoV-2 | | SLLVLVQSTQWSLFFFLYEN |
| | nsp6-FL | | AFLPFAMGIIAMSAFAMMFV |
| | | | KHKHAFLCLFLLPSLATVAY |
| | | | FNMVYMPASWVMRIMTWLDM |
| | | | VDTSLSGFKLKDCVMYASAV |
| | | | VLLILMTARTVYDDGARRVW |
| | | | TLMNVLTLVYKVYYGNALDQ |
| | | | AISMWALIISVTSNYSGVVT |
| | | | TVMFLARGIVFMCVEYCPIF |
| | | | FITGNTLQCIMLVYCFLGYF |
| | | | CTCYFGLFCLLNRYFRLTLG |
| | | | VYDYLVSTQEFRYMNSQGLL |
| | | | PPKNSIDAFKLNIKLLGVGG |
| | | | KPCIKVATVQ |
| | SARS- | 1-83 | SKMSDVKCTSVVLLSVLQQL |
| | CoV-2 | | RVESSSKLWAQCVQLHNDIL |
| | nsp7-FL | | LAKDTTEAFEKMVSLLSVLL |
| | | | SMQGAVDINKLCEEMLDNRA |
| | | | TLQ |

¹aa positions based on SARS-CoV-2 reference genome (NC-06577.2; Wuhan strain) and reference nsp6 protein (YP_009725302.1) and nsp7 protein (YP_009725303.1).
²For conjugation to polyionic VLPS, the peptide/protein antigens have a N-terminal TAG and AAYY proteolytic processing sequence.

TABLE 9
SARS-CoV-2 antigens of nsp12-1
nonstructural protein fragment

| | | aa- | |
|---|---|---|---|
| Immunogen | Seq ID | position¹ | Amino acid sequence² |
| Short | SARS- | 126-153 | DLVYALRHFDEGNCDTLKEI |
| peptide | CoV-2 | | LVTYNCCD |
| | nsp12- | | |
| | 1-S1 | | |
| | SARS- | 144-171 | EILVTYNCCDDDYFNKKDWY |
| | CoV-2 | | DFVENPDI |
| | nsp12- | | |
| | 1-S2 | | |
| | SARS- | 162-189 | WYDFVENPDILRVYANLGER |
| | CoV-2 | | VRQALLKT |
| | nsp12- | | |
| | 1-S3 | | |
| | SARS- | 180-207 | ERVRQALLKTVQFCDAMRNA |
| | CoV-2 | | GIVGVLTL |
| | nsp12- | | |
| | 1-S4 | | |
| | SARS- | 198-225 | NAGIVGVLTLDNQDLNGNWY |
| | CoV-2 | | DFGDFIQT |
| | nsp12- | | |
| | 1-S5 | | |
| | SARS- | 216-243 | WYDFGDFIQTTPGSGVPVVD |
| | CoV-2 | | SYYSLLMP |
| | nsp12- | | |
| | 1-S6 | | |
| | SARS- | 234-261 | VDSYYSLLMPILTLTRALTA |
| | CoV-2 | | ESHVDTDL |
| | nsp12- | | |
| | 1-S7 | | |
| | SARS- | 252-280 | TAESHVDTDLTKPYIKWDLL |
| | CoV-2 | | KYDFTEERL |
| | nsp12- | | |
| | 1-S8 | | |
| | SARS- | 271-299 | LKYDFTEERLKLFDRYFKYW |
| | CoV-2 | | DQTYHPNCV |
| | nsp12- | | |
| | 1-S9 | | |
| | SARS- | 290-317 | WDQTYHPNCVNCLDDRCILH |
| | CoV-2 | | CANFNVLF |
| | nsp12- | | |
| | 1-S10 | | |
| | SARS- | 308-336 | LHCANFNVLFSTVFPPTSFG |
| | CoV-2 | | PLVRKIFVD |
| | nsp12- | | |
| | 1-S11 | | |
| | SARS- | 327-355 | GPLVRKIFVDGVPFVVSTGY |
| | CoV-2 | | HFRELGVVH |
| | nsp12- | | |
| | 1-S12 | | |
| | SARS- | 346-375 | YHFRELGVVHNQDVNLHSSR |
| | CoV-2 | | LSFKELLVYA |
| | nsp12- | | |
| | 1-S13 | | |
| Extended | SARS- | 126-175 | DLVYALRHFDEGNCDTLKEI |
| peptide | CoV-2 | | LVTYNCCDDDYFNKKDWYDF |
| | nsp12- | | VENPDILRVY |
| | 1-E1 | | |
| | SARS- | 166-215 | VENPDILRVYANLGERVRQA |
| | CoV-2 | | LLKTVQFCDAMRNAGIVGVL |
| | nsp12- | | TLDNQDLNGN |
| | 1-E2 | | |
| | SARS- | 206-255 | TLDNQDLNGNWYDFGDFIQT |
| | CoV-2 | | TPGSGVPVVDSYYSLLMPIL |
| | nsp12- | | TLTRALTAES |
| | 1-E3 | | |
| | SARS- | 246-295 | TLTRALTAESHVDTDLTKPY |
| | CoV-2 | | IKWDLLKYDFTEERLKLFDR |
| | nsp12- | | YFKYWDQTYH |
| | 1-E4 | | |
| | SARS- | 286-335 | YFKYWDQTYHPNCVNCLDDR |
| | CoV-2 | | CILHCANFNVLFSTVFPPTS |
| | nsp12- | | FGPLVRKIFV |
| | 1-E5 | | |
| | SARS- | 326-375 | FGPLVRKIFVDGVPFVVSTG |
| | CoV-2 | | YHFRELGVVHNQDVNLHSSR |
| | nsp12- | | LSFKELLVYA |
| | 1-E6 | | |
| Short | SARS- | 126-255 | DLVYALRHFDEGNCDTLKEI |
| protein | CoV-2 | | LVTYNCCDDDYFNKKDWYDF |
| | nsp12- | | VENPDILRVYANLGERVRQA |
| | 1-SP1 | | LLKTVQFCDAMRNAGIVGVL |
| | | | TLDNQDLNGNWYDFGDFIQT |
| | | | TPGSGVPVVDSYYSLLMPIL |
| | | | TLTRALTAES |
| | SARS- | 246-375 | TLTRALTAESHVDTDLTKPY |
| | CoV-2 | | IKWDLLKYDFTEERLKLFDR |
| | nsp12- | | YFKYWDQTYHPNCVNCLDDR |
| | 1-SP2 | | CILHCANFNVLFSTVFPPTS |
| | | | FGPLVRKIFVDGVPFVVSTG |
| | | | YHFRELGVVHNQDVNLHSSR |
| | | | LSFKELLVYA |
| Full | SARS- | 126-375 | DLVYALRHFDEGNCDTLKEI |
| length | CoV-2 | | LVTYNCCDDDYFNKKDWYDF |
| antigen | nsp12- | | VENPDILRVYANLGERVRQA |
| | 1-FL | | LLKTVQFCDAMRNAGIVGVL |
| | | | TLDNQDLNGNWYDFGDFIQT |
| | | | TPGSGVPVVDSYYSLLMPIL |
| | | | TLTRALTAESHVDTDLTKPY |
| | | | IKWDLLKYDFTEERLKLFDR |
| | | | YFKYWDQTYHPNCVNCLDDR |
| | | | CILHCANFNVLFSTVFPPTS |
| | | | FGPLVRKIFVDGVPFVVSTG |
| | | | YHFRELGVVHNQDVNLHSSR |
| | | | LSFKELLVYA |

¹aa positions based on SARS-CoV-2 reference genome (NC-06577.2; Wuhan strain) and reference nsp12 protein (YP_009725307.1).
²For conjugation to polyionic VLPS, the peptide/protein antigens have a N-terminal TAG and AAYY proteolytic processing sequence.

TABLE 10
SARS-CoV-2 antigens of nsp12-2
nonstructural protein fragment

| | | aa- | |
|---|---|---|---|
| Immunogen | Seq ID | position | Amino acid sequence |
| Short | SARS- | 520-548 | SYEDQDALFAYTKRNVIPT |
| peptide | CoV-2 | | ITQMNLKYAI |
| | nsp12- | | |
| | 2-S1 | | |
| | SARS- | 538-566 | TITQMNLKYAISAKNRART |
| | CoV-2 | | VAGVSICSTM |
| | nsp12- | | |
| | 2-S2 | | |
| | SARS- | 557-586 | VAGVSICSTMNRQFHQKLL |
| | CoV-2 | | KSIAATRGAT |
| | nsp12- | | |
| | 2-S3 | | |
| | SARS- | 576-604 | LKSIAATRGATVVIGTSKF |
| | CoV-2 | | YGGWHNMLKT |
| | nsp12- | | |
| | 2-S4 | | |
| | SARS- | 595-624 | YGGWHNMLKTVYSDVENPH |
| | CoV-2 | | LMGWDYPKCDR |
| | nsp12- | | |
| | 2-S5 | | |
| | SARS- | 615-643 | MGWDYPKCDRAMPNMLRIM |
| | CoV-2 | | ASLVLARKHT |
| | nsp12- | | |
| | 2-S6 | | |
| | SARS- | 634-662 | ASLVLARKHTTCCSLSHRF |
| | CoV-2 | | YRLANECAQV |
| | nsp12- | | |
| | 2-S7 | | |
| | SARS- | 653-683 | YRLANECAQVLSEMVMCGG |
| | CoV-2 | | SLYVKPGGTSSG |
| | nsp12- | | |
| | 2-S8 | | |
| | SARS- | 674-704 | YVKPGGTSSGDATTAYANS |
| | CoV-2 | | VFNICQAVTANV |
| | nsp12- | | |
| | 2-S9 | | |
| | SARS- | 695-724 | NICQAVTANVNALLSTDGN |
| | CoV-2 | | KIADKYVRNLQ |
| | nsp12- | | |
| | 2-S10 | | |
| | SARS- | 715-744 | IADKYVRNLQHRLYECLYR |
| | CoV-2 | | NRDVDTDFVNE |
| | nsp12- | | |
| | 2-S11 | | |
| | SARS- | 735-764 | RDVDTDFVNEFYAYLRKHF |
| | CoV-2 | | SMMILSDDAVV |
| | nsp12- | | |
| | 2-S12 | | |
| | SARS- | 755-784 | MMILSDDAVVCFNSTYASQ |
| | CoV-2 | | GLVASIKNFKS |
| | nsp12- | | |
| | 2-S13 | | |
| | SARS- | 775-804 | LVASIKNFKSVLYYQNNVF |
| | CoV-2 | | MSEAKCWTETD |
| | nsp12- | | |
| | 2-S14 | | |
| | SARS- | 795-824 | SEAKCWTETDLTKGPHEFC |
| | CoV-2 | | SQHTMLVKQGD |
| | nsp12- | | |
| | 2-S15 | | |
| | SARS- | 815-844 | QHTMLVKQGDDYVYLPYPD |
| | CoV-2 | | PSRILGAGCFV |
| | nsp12- | | |
| | 2-S16 | | |
| | SARS- | 835-863 | SRILGAGCFVDDIVKTDGT |
| | CoV-2 | | LMIERFVSLA |
| | nsp12- | | |
| | 2-S17 | | |
| | SARS- | 854-882 | LMIERFVSLAIDAYPLTKH |
| | CoV-2 | | PNQEYADVFH |
| | nsp12- | | |
| | 2-S18 | | |
| | SARS- | 873-901 | PNQEYADVFHLYLQYIRKL |
| | CoV-2 | | HDELTGHMLD |
| | nsp12- | | |
| | 2-S19 | | |
| | SARS- | 892-920 | HDELTGHMLDMYSVMLTND |
| | CoV-2 | | NTSRYWEPEF |
| | nsp12- | | |
| | 2-S20 | | |
| Extended | SARS- | 520-498 | SYEDQDALFAYTKRNVIPT |
| peptide | CoV-2 | | ITQMNLKYAISAKNRARTV |
| | nsp12- | | AGVSICSTMTN |
| | 2-E1 | | |
| | SARS- | 559-607 | GVSICSTMTNRQFHQKLLK |
| | CoV-2 | | SIAATRGATVVIGTSKFYG |
| | nsp12- | | GWHNMLKTVYS |
| | 2-E2 | | |
| | SARS- | 598-646 | WHNMLKTVYSDVENPHLMG |
| | CoV-2 | | WDYPKCDRAMPNMLRIMAS |
| | nsp12- | | LVLARKHTTCC |
| | 2-E3 | | |
| | SARS- | 637-685 | VLARKHTTCCSLSHRFYRL |
| | CoV-2 | | ANECAQVLSEMVMCGGSLY |
| | nsp12- | | VKPGGTSSGDA |
| | 2-E4 | | |
| | SARS- | 676-724 | KPGGTSSGDATTAYANSVF |
| | CoV-2 | | NICQAVTANVNALLSTDGN |
| | nsp12- | | KIADKYVRNLQ |
| | 2-E5 | | |
| | SARS- | 715-763 | IADKYVRNLQHRLYECLYR |
| | CoV-2 | | NRDVDTDFVNEFYAYLRKH |
| | nsp12- | | FSMMILSDDAV |
| | 2-E6 | | |
| | SARS- | 754-802 | SMMILSDDAVVCFNSTYAS |
| | CoV-2 | | QGLVASIKNFKSVLYYQNN |
| | nsp12- | | VFMSEAKCWTE |
| | 2-E7 | | |
| | SARS- | 793-841 | FMSEAKCWTETDLTKGPHE |
| | CoV-2 | | FCSQHTMLVKQGDDYVYLP |
| | nsp12- | | YPDPSRILGAG |
| | 2-E8 | | |
| | SARS- | 832-880 | PDPSRILGAGCFVDDIVKT |
| | CoV-2 | | DGTLMIERFVSLAIDAYPL |
| | nsp12- | | TKHPNQEYADV |
| | 2-E9 | | |
| | SARS- | 871-920 | KHPNQEYADVFHLYLQYIR |
| | CoV-2 | | KLHDELTGHMLDMYSVMLT |
| | nsp12- | | NDNTSRYWEPEF |
| | 2-E10 | | |
| Short | SARS- | 520-629 | SYEDQDALFAYTKRNVIPT |
| protein | CoV-2 | | ITQMNLKYAISAKNRARTV |
| | nsp12- | | AGVSICSTMTNRQFHQKLL |
| | 2-SP1 | | KSIAATRGATVVIGTSKFY |
| | | | GGWHNMLKTVYSDVENPHL |
| | | | MGWDYPKCDRAMPNM |
| | SARS- | 619-727 | YPKCDRAMPNMLRIMASLV |
| | CoV-2 | | LARKHTTCCSLSHRFYRLA |
| | nsp12- | | NECAQVLSEMVMCGGSLYV |
| | 2-SP2 | | KPGGTSSGDATTAYANSVF |
| | | | NICQAVTANVNALLSTDGN |
| | | | KIADKYVRNLQHRL |
| | SARS- | 717-826 | DKYVRNLQHRLYECLYRNR |
| | CoV-2 | | DVDTDFVNEFYAYLRKHFS |
| | nsp12- | | MMILSDDAVVCFNSTYASQ |
| | 2-SP3 | | GLVASIKNFKSVLYYQNNV |
| | | | FMSEAKCWTETDLTKGPHE |
| | | | FCSQHTMLVKQGDDY |
| | SARS- | 816-920 | HTMLVKQGDDYVYLPYPDP |
| | CoV-2 | | SRILGAGCFVDDIVKTDGT |
| | nsp12- | | LMIERFVSLAIDAYPLTKH |
| | 2-SP4 | | PNQEYADVFHLYLQYIRKL |
| | | | HDELTGHMLDMYSVMLTND |
| | | | NTSRYWEPEF |
| Full | SARS- | 520-920 | SYEDQDALFAYTKRNVIPT |
| length | CoV-2 | | ITQMNLKYAISAKNRARTV |
| antigen | nsp12- | | AGVSICSTMTNRQFHQKLL |
| | 2-FL | | KSIAATRGATVVIGTSKFY |
| | | | GGWHNMLKTVYSDVENPHL |
| | | | MGWDYPKCDRAMPNMLRIM |
| | | | ASLVLARKHTTCCSLSHRF |
| | | | YRLANECAQVLSEMVMCGG |
| | | | SLYVKPGGTSSGDATTAYA |
| | | | NSVFNICQAVTANVNALLS |
| | | | TDGNKIADKYVRNLQHRLY |
| | | | ECLYRNRDVDTDFVNEFYA |
| | | | YLRKHFSMMILSDDAVVCF |
| | | | NSTYASQGLVASIKNFKSV |
| | | | LYYQNNVFMSEAKCWTETD |
| | | | LTKGPHEFCSQHTMLVKQG |
| | | | DDYVYLPYPDPSRILGAGC |
| | | | FVDDIVKTDGTLMIERFVS |
| | | | LAIDAYPLTKHPNQEYADV |
| | | | FHLYLQYIRKLHDELTGHM |
| | | | LDMYSVMLTNDNTSRYWEP |
| | | | EF |

¹aa positions based on SARS-CoV-2 reference genome (NC-06577.2; Wuhan strain) and reference nsp12 protein (YP_009725307.1).
²For conjugation to polyionic VLPS, the peptide/protein antigens have a N-terminal TAG and AAYY proteolytic processing sequence.

TABLE 11
OC43 and HKU1 antigens
of the M structural protein

| | | aa- | |
|---|---|---|---|
| Immunogen | Seq ID | position¹ | Amino acid sequence² |
| Short | OC43-M-S1 | aa14-42 | TADEAIKFLKEWNFSLGIIL |
| peptides | | | LFITIILQF |
| | OC43-M-S2 | aa33-60 | LLFITIIILQFGYTSRSMFV |
| | | | YVIKMIILW |
| | OC43-M-S3 | aa51-79 | VYVIKMIILWLMWPLTIILT |
| | | | IFNCVYALN |
| | OC43-M-S4 | aa70-98 | TIFNCVYALNNVYLGLSIVF |
| | | | TIVAIIMWI |
| | OC43-M-S5 | aa89-117 | FTIVAIIMWIVYFVNSIRLF |
| | | | IRTGSFWSF |
| | OC43-M-S6 | aa108-136 | FIRTGSFWSFNPETNNLMCI |
| | | | DMKGTMYVR |
| | OC43-M-S7 | aa127-154 | IDMKGTMYVRPIIEDYHTLT |
| | | | VTIIRGHL |
| | OC43-M-S8 | aa145-172 | LTVTIIRGHLYIQGIKLGTG |
| | | | YSLADLPA |
| | OC43-M-S9 | aa163-190 | TGYSLADLPAYMTVAKVTHL |
| | | | CTYKRGFL |
| | OC43-M- | aa181-208 | HLCTYKRGFLDRISDTSGFA |
| | S10 | | VYVKSKVG |
| | OC43-M- | aa199-226 | FAVYVKSKVGNYRLPSTQKG |
| | S11 | | SGMDTALL |
| | HKU1-M-S1 | aa75-97 | NNAFLAFSIVFTIISIVIWI |
| | | | LYF |
| | HKU1-M-S2 | aa174-200 | KVQVLCTYKRAFLDKLDVNS |
| | | | GFAVFVK |
| Extended | OC43-M-E1 | aa14-57 | TADEAIKFLKEWNFSLGIIL |
| peptides | | | LFITIILQFGYTSRSMFVYV |
| | | | IKMI |
| | OC43-M-E2 | aa48-91 | SMFVYVIKMIILWLMWPLTI |
| | | | ILTIFNCVYALNNVYLGLSI |
| | | | VFTI |
| | OC43-M-E3 | aa82-125 | YLGLSIVFTIVAIIMWIVYF |
| | | | VNSIRLFIRTGSFWSFNPET |
| | | | NNLM |
| | OC43-M-E4 | aa116-159 | SFNPETNNLMCIDMKGTMFV |
| | | | RPIIEDYHTLTVTIIRGHLY |
| | | | IQGI |
| | OC43-M-E5 | aa150-194 | IRGHLYIQGIKLGTGYSLAD |
| | | | LPAYMTVAKVTYLCTYKRGF |
| | | | LDKIS |
| | OC43-M-E6 | aa185-226 | YKRGFLDKISDTSGFAVYVK |
| | | | SKVGNYRLPSTQKGSGMDTA |
| | | | LL |
| | HKU1-M-E1 | aa75-97/ | NNAFLAFSIVFTIISIVIWI |
| | | 174-200 | LYF/KVQVLCTYKRAFLDKL |
| | | | DVNSGFAVFVK |
| Short | OC43-M-P1 | aa14-151 | TADEAIKFLKEWNFSLGIIL |
| proteins | | | LFITIILQFGYTSRSMFVYV |
| | | | IKMIILWLMWPLTIILTIFN |
| | | | CVYALNNVYLGLSIVFTIVA |
| | | | IIMWIVYFVNSIRLFIRTGS |
| | | | FWSFNPETNNLMCIDMKGTM |
| | | | YVRPIIEDYHTLTVTIIR |
| | OC43// | aa141-226// | DYHTLTVTIIRGHLYIQGIK |
| | HKU1- | aa75-97/ | LGTGYSLADLPAYMTVAKVT |
| | M-P2 | aa174-200 | HLCTYKRGFLDRISDTSGFA |
| | | | VYVKSKVGNYRLPSTQKGSG |
| | | | MDTALL/NNAFLAFSIVFTI |
| | | | ISIVIWILYF/KVQVLCTYK |
| | | | RAFLDKLDVNSGFAVFVK |
| Full | OC43-M// | aa14-226// | TADEAIKFLKEWNFSLGIIL |
| length | HKU1-M- | aa75-97/ | LFITIILQFGYTSRSMFVYV |
| | variant- | aa174-200 | IKMIILWLMWPLTIILTIFN |
| | FL | | CVYALNNVYLGLSIVFTIVA |
| | (263 aa) | | IIMWIVYFVNSIRLFIRTGS |
| | | | FWSFNPETNNLMCIDMKGTM |
| | | | YVRPIIEDYHTLTVTIIRGH |
| | | | LYIQGIKLGTGYSLADLPAY |
| | | | MTVAKVTHLCTYKRGFLDRI |
| | | | SDTSGFAVYVKSKVGNYRLP |
| | | | STQKGSGMDTALL//NNAFL |
| | | | AFSIVFTIISIVIWILYF/K |
| | | | VQVLCTYKRAFLDKLDVNSG |
| | | | FAVFVK |

¹aa positions based on OC43 reference M protein sequences, YP_009555244.1, and HKU1 reference M protein sequence, YP_173241.1.
²For conjugation to polyionic VLPS, the peptide/protein antigens have a N-terminal TAG and AAYY proteolytic processing sequence.
/ indicates break in native amino acid sequence
// indicates break between OC43 amino acid sequences and HKU1 amino acid sequences.

TABLE 12
OC43 and HKU1 antigens of N structural protein

| | | aa | |
|---|---|---|---|
| Immunogen | Seq ID | positions¹ | Amino acid sequence² |
| Short | OC43-N- | aa64-88 | SWFSGITQFQKGKEFEFVEGQGVPI |
| peptides | S1 | | |
| | OC43-N- | aa99-131 | GYWYRHNRRSFKTADGNQRQLLPRWYF |
| | S2 | | YYLGTG |
| | OC43-N- | aa122-155 | RWYFYYLGTGPHAKDQYGDIDGVYWVA |
| | S3 | | SNQAVD |
| | OC43-N- | aa146-177 | YWVASNQADVNTPADIVDRDPSSDEAIP |
| | S4 | | TRFP |
| | OC43-N- | aa168-200 | SDEAIPTRFPPGTVLPQGYYIEGSGRSAPN |
| | S5 | | SRS |
| | OC43-N- | aa191-223 | SGRSAPNSRSTSRTSSRASSAGSRSRANSG |
| | S6 | | NRT |
| | OC43-N- | aa214-245 | RSRANSGNRTPTSGVTPDMADQIASLVL |
| | S7 | | AKLG |
| | OC43-N- | aa236-265 | IASLVLAKLGKDATKPQQVTKHTAKEVR |
| | S8 | | QK |
| | OC43-N- | aa270-302 | PRQKRSPNKQCTVQQCFGKRGPNQNFG |
| | S9 | | GGEMLK |
| | OC43-N- | aa293-325 | QNFGGGEMLKLGTSDPQFPILAELAPTA |
| | S10 | | GAFFF |
| | OC43-N- | aa316-340/ | LAPTAGAFFFGSRLELAKVQNLSGN/ELR |
| | S11 | aa350-358 | YNGAIR |
| | OC43-N- | aa350-381 | ELRYNGAIRFDSTLSGFETIMKVLNENLN |
| | S12 | | AYQ |
| | HKU1-N- | aa131-156 | PYANASYGESLEGVFWVANHQADTST |
| | S1 | | |
| | HKU1-N- | aa147-170 | VANHQADTSTPSDVSSRDPTTQEA |
| | S2 | | |
| | HKU1-N- | aa258-289 | RPGSRSQSRGPNNRSLSRSNSNFRHSDSIV |
| | S3 | | KP |
| | HKU1-N- | aa325-341 | SKLDLVKRDSEADSPVK |
| | S4 | | |
| Extended | OC43-N-1 | aa64-88/ | SWFSGITQFQKGKEFEFVEGQGVPI/ |
| peptides | | aa99-119 | GYWYRHNRRSFKTADGNQRQL |
| | OC43-N-2 | aa110-155 | KTADGNQRQLLPRWYFYYLGTGPHAKD |
| | | | QYGTDIDGVYWVASNQADV |
| | OC43-N-3 | aa146-191 | YWVASNQADVNTPADIVDRDPSSDEAIP |
| | | | TRFPPGTVLPQGYYIEGS |
| | OC43-N-4 | aa182-224 | LPQGYYIEGSGRSAPNSRSTSRTSRTSSRAS |
| | | | SAGSRSRANSGNRTP |
| | OC43-N-5 | aa215-260 | SRANSGNRTPTSGVTPDMADQIASLVLA |
| | | | KLGKDATKPQQVTKHTAK |
| | OC43-N-6 | aa251-265/ | PQQVTKHTAKEVRQK/ |
| | | aa270-300 | PRQKRSPNKQCTVQQCFGKRGPNQNFG |
| | | | GGEM |
| | OC43-N-7 | aa291-336 | PNQNFGGGEMLKLGTSDPQFPILAELAP |
| | | | TAGAFFFGSRLELAKVQN |
| | OC43-N-8 | aa327-340/ | SRLELAKVQNLSGN/ELRYNGAIRFDSTL |
| | | aa350-381 | SGFETIMKVLNENLNAYQ |
| | KHU1-N- | aa131-170 | PYANASYGESLEGVFWVANHQADTSTPS |
| | 1 | | DVSSRDPTTQEA |
| | KHU1-N- | aa198-229/ | RPGSRSQSRGPNNRSLSRSNSNFRHSDSIV |
| | 2 | aa325-341 | KP/SKLDLVKRDSEADSPVK |
| Short | OC43-N- | aa64-88/ | SWFSGITQFQKGKEFEFVEGQGVPI/ |
| protein | P1 | aa99-184 | GYWYRHNRRSFKTADGNQRQLLPRWYF |
| | | | YYLGTGPHAKDQYGT |
| | | | DIDGVYWVASNQADVNTPADIVDRDPS |
| | | | SDEAIPTRFPPGTVLPQ |
| | OC43-N- | aa174-265/ | TRFPPGTVLPQGYYIEGSGRSAPNSRSTSR |
| | P2 | 270-283 | TSSRASSAGSRSRANSGNRTPTSGVTPDM |
| | | | ADQIASLVLAKLGKDATKPQQVTKHTA |
| | | | KEVRQK/PRQKRSPNKQCTVQ |
| | OC43-N- | aa273-381 | KRSPNKQCTVQQCFGKRGPNQNFGGGE |
| | P3 | | MLKGTSDPQFPILAELAPTAGAFFFGSRL |
| | | | ELAKVQNLSGNELRYNGAIRFDSTLSGFE |
| | | | TIMKVLNENLNAYQ |
| | HKU-N- | aa131-170/ | PYANASYGESLEGVFWVANHQADTSTPS |
| | P1 | 198-229/ | DVSSRDPTTQEA/ |
| | | 325-341 | RPGSRSQSRGPNNRSLSRSNSNFRHSDSIV |
| | | | KP/SKLDLVKRDSEADSPVK |
| Full | OC43- | aa64-88/ | SWFSGITQFQKGKEFEFVEGQGVPI/ |
| length | N/HKU1- | aa99-265/ | GYWYRHNRRSFKTADGNQRQLLPRWYF |
| | N-variant- | aa270-381// | YYLGTGPHAKDQYGDIDGVYWVASNQA |
| | FL | aa131-170/ | DVNTPADIVDRDPSSDEAIPTRFPPGTVL |
| | (383 aa) | 198-229/ | PQGYYIEGSGRSAPNSRSTSRTSSRASSAG |
| | | 325-341 | SRSRANSGNRTPTSGVTPDMADQIASLV |
| | | | LAKLGKDATKPQQVTKHTAKEVRQK/ |
| | | | PRQKRSPNKQCTVQQCFGKRGPNQNFG |
| | | | GGEMLKLGTSDPQFPILAELAPTAGAFFF |
| | | | GSRLELAKVQNLSGNELRYNGAIRFDSTL |
| | | | SGFETIMKVLNENLNAYQ// |
| | | | PYANASYGESLEGVFWVANHQADTSTPS |
| | | | DVSSRDPTTQEA/ |
| | | | RPGSRSQSRGPNNRSLSRSNSNFRHSDSIV |
| | | | KP/SKLDLVKRDSEADSPVK |

¹aa positions based on OC43 reference N protein sequences, YP_009555245.1, and HKU1 reference N protein sequence, YP_173242.1.
²For conjugation to polyionic VLPS, the peptide/protein antigens have a N-terminal TAG and AAYY proteolytic processing sequence.
/ indicates break in native amino acid sequence
// indicates break between OC43 amino acid sequences and HKU1 amino acid sequences.

TABLE 13
OC43 and HKU1 S2 antigens of the S2 structural protein

| | | aa | |
|---|---|---|---|
| Immunogen | Seq ID | positions¹ | Amino acid sequence² |
| Short | OC43-S2- | aa898-929 | SKASSRSAIEDLLFDKVKLSDVGFVEAY |
| peptide | S1 | | NNCT |
| | OC43-S2- | aa920-951 | GFVEAYNNCTGGAEIRDLICVQSYKGI |
| | S2 | | KVLPP |
| | OC43-S2- | aa943-973 | SYKGIKVLPPLLSENQISGYTLAATSASL |
| | S3 | | FPP |
| | OC43-S2- | aa964-995 | AATSASLFPPWTAAAGVPFYLNVQYRI |
| | S4 | | NGLGV |
| | OC43-S2- | aa986-1017 | VQYRINGLGVTMDVLSQNQKLIANAF |
| | S5 | | NNALYA |
| | OC43-S2- | aa1008-1039 | ANAFNNALYAIQEGFDATNSALVKIQ |
| | S6 | | AVVNAN |
| | OC43-S2- | aa1030-1061 | VKIQAVVNANAEALNNLLQQLSNRFG |
| | S7 | | AISASL |
| | OC43-S2- | aa1052-1084 | NRFGAISASLQEILSRLDALEAEAQIDRL |
| | S8 | | INGR |
| | OC43-S2- | aa1075-1107 | AQIDRLINGRLTALNAYVSQQLSDSTLV |
| | S9 | | KFSAA |
| | OC43-S2- | aa1098-1130 | DSTLVKFSAAQAMEKVNECVKSQSSRI |
| | S10 | | NFCGNG |
| | OC43-S2- | aa1120-1153 | QSSRINFCGNGNHI |
| | S11 | | ISLVQNAPYGLYFIHFSYVP/ |
| | OC43-S2- | aa1228-1258 | PNLPDFKEELDQWFKNQTSVAPDLSLD |
| | S12 | | YINVT |
| | OC43-S2- | aa1250-1281 | DLSLDYINVTFLDLQVEMNRLQEAIKV |
| | S13 | | LNQSY |
| | OC43-S2- | aa1272-1302 | EAIKVLNQSYINLKDIGTYEYYVKWPW |
| | S14 | | YVWL |
| | HKU1-S2- | aa1229-1258 | PKLSDFESELSHWFKNQTSIAPNLTLNL |
| | S1 | | HT |
| | HKU1-S2- | aa1249-1280 | APNLTLNLHTINATFLDLYYEMNLIQES |
| | S2 | | IKSL |
| Extended | OC43-S2- | aa898-942 | SKASSRSAIEDLLFDKVKLSDVGFVEAY |
| peptide | E1 | | NNCTGGAEIRDLICVQS |
| | OC43-S2- | aa933-978 | EIRDLICVQSYKGIKVLPPLLSENQISGY |
| | E2 | | TLAATSASLFPPWTAAA |
| | OC43-S2- | aa969-1013 | SLFPPWTAAAGVPFYLNVQYRINGLGV |
| | E3 | | TMDVLSQNQKLIANAFNN |
| | OC43-S2- | aa1004-1048 | QKLIANAFNNALYAIQEGFDATNSALV |
| | E4 | | KIQAVVNANAEALNNLLQ |
| | OC43-S2- | aa1039-1083 | NAEALNNLLQQLSNRFGAISASLQEILS |
| | E5 | | RLDALEAEAQIDRLING |
| | OC43-S2- | aa1074-1119 | EAQIDRLINGRLTALNAYVSQQLSDSTL |
| | E6 | | VKFSAAQAMEKVNECKS |
| | OC43-S2- | aa1109-1153 | AMEKVNECVKSQSSRINFCGNGNHIIS |
| | E7 | | LVQNAPYGLYFIHFSYVP/ |
| | OC43-S2- | aa1228-1269 | PNLPDFKEELDQWFKNQTSVAPDLSLD |
| | E8 | | YINVTFLDLQVEMNR |
| | OC43-S2- | aa1260-1302 | FLDLQVEMNRLQEAIKVLNQSYINLKD |
| | E9 | | IGTYEYYVKWPWYVWL |
| | HKU1-S2- | aa1229-1280 | PKLSDFESELSHWFKNQTSIAPNLTLNL |
| | E1 | | HTINATFL MNLIQESIKSL |
| Short | OC43-S2- | aa898-1033 | SKASSRSAIEDLLFDKVKLSDVGFVEAY |
| protein | P1 | | NNCTGGAEIRDLICVQSYKGIKVLPPLL |
| | | | SENQISGYTLAATSASLFPPWTAAAGV |
| | | | PFYLNVQYRINGLGVTMDVLSQNQKLI |
| | | | ANAFNNALYAIQEGFDATNSALVKIQ |
| | OC43-S2- | aa1022-1153 | FDATNSALVKIQAVVNANAEALNNLL |
| | P2 | | QQLSNRFGAISASLQEILSRLDALEAEA |
| | | | QIDRLINGRLTALNAYVSQQLSDSTLVK |
| | | | FSAAQAMEKVNECVKSQSSRINFCGNG |
| | | | NHIISLVQNAPYGLYFIHFSYVP/ |
| | OC43/HK | aa1228-1302// | PNLPDFKEELDQWFKNQTSVAPDLSDY |
| | U1- | 1229-1280 | INVFLDLQVEMNRLQEAIKVLNQSYIN |
| | variant-S2- | | LKDIGTYEYYVKWPWYVWL//PKLSDF |
| | P3 | | ESELSHWFKNQTSIAPNLTLNLHTINAT |
| | | | FLMNLIQESIKSL |

¹aa positions based on OC43 reference Spike protein sequences, YP_009555241.1, and HKU1 reference spike protein sequence, YP_173238.1.
²For conjugation to polyionic VLPS, the peptide/protein antigens have a N-terminal TAG and AAYY proteolytic processing sequence.
/ indicates break in native amino acid sequence
// indicates break between OC43 amino acid sequences and HKU1 amino acid sequences

TABLE 14
OC43 antigens of nonstructural proteins nsp3 and nsp4

| Immunogen | Seq ID | aa positions¹ | Amino acid sequence² |
|---|---|---|---|
| Short | OC43- | aa2368-2399 | FMRFYIIIASFIKLFSLFRHVAYGCSKSGCLF |
| peptide | nsp3-S1 | | |
| | OC43- | aa2390-2422 | YGCSKSGCLFYKRNRSLRVKCSTIVGGMIRYY |
| | nsp3-S2 | | |
| | OC43- | aa2413-2442 | TIVGGMIRYYDVMANGGTGFCSKHQWNCID |
| | nsp3-S3 | | |
| | OC43- | aa2433-2463 | CSKHQWNCIDCDSYKPGNTFITVEAALDLSK |
| | nsp3-S4 | | |
| | OC43- | aa2454-2473/ | TVEAALDLSKELKRPIQPTD/NAAVFYAQSLFR |
| | nsp3-S5 | aa2542-2553 | |
| | OC43- | aa2544-2574 | AVFYAQSLFRPILMVDKNLITTANTGTSVTE |
| | nsp3-S6 | | |
| | OC43- | aa2565-2595 | TANTGTSVTETMFDVYVDTFLSMFDVDKKSL |
| | nsp3-S7 | | |
| | OC43- | aa2586-2602/ | SMFDVDKKSLNALIATA/ELTDESCNNLVPTYL |
| | nsp3-S8 | aa2651-2665 | |
| | OC43- | aa2656-2686 | SCNNLVPTYLKSDNIVAADLGVLIQNSAKHV |
| | nsp3-S9 | | |
| | OC43- | aa2677-2705 | VLIQNSAKHVQGNVAKIAGVSCIWSVDAF |
| | nsp3-S10 | | |
| Extended | OC43- | aa2368-2409 | FMRFYIIIASFIKLFSLFRHVAYGCSKSGCLFCYKRN |
| peptide | nsp3-E1 | | RSLRV |
| | OC43- | aa2400-2442 | CYRNRSLRVKCSTIVGGMIRYYDVMANGGTGFCSK |
| | nsp3-E2 | | HQWNCID |
| | OC43- | aa2433-2473 | CSKHQWNCIDCDSYKPGNTFITVEAALDLSKELKR |
| | nsp3-E3 | | PIQPTD/ |
| | OC43- | aa2542-2585 | NAAVFYAQSLFRPILMVDKNLITTANTGTSVTETM |
| | nsp3-E4 | | FDVYVDTFL |
| | OC43- | aa2576-2602/ | MFDVYVDTFLSMFDVDKKSLNALIATA/ |
| | nsp3-E5 | aa2651-2673 | ELTDESCNNLVPTYLKSDNIVAA |
| | OC43- | aa2664-2705 | YLKSDNIVAADLGVLIQNSAKHVQGNVAKIAGVS |
| | nsp3-E6 | | CIWSVDAF |
| Short | OC43- | aa2368-2473 | FMRFYIIIASFIKLFSLFRHVAYGCSKSGCLFCYKRN |
| protein | nsp3-P1 | | RSLRVKCSTI |
| | | | VGGMIRYYDVMANGGTGFCSKHQWNCIDCDSYK |
| | | | PGNTFITVEAALDLSKELKRPIQPTD/ |
| | OC43- | aa2542-2602/ | NAAVFYAQSLFRPILMVDKNLITTANTGTSVTETM |
| | nsp3-P2 | aa2651-2705 | FDVYVDTFLS |
| | | | MFDVDKKSLNALIATA / |
| | | | ELTDESCNNLVPTYLKSDNIVAADLGVLIQNSAKH |
| | | | VQGNVAKIAG |
| | | | VSCIWSVDAF |
| Full | OC43- | aa2368-2473/ | FMRFYIIIASFIKLFSLFRHVAYGCSKSGCLFCYKRN |
| length | nsp3-FL | aa2542-2602/ | RSLRVKCSTIV |
| | | aa2651-2705 | GGMIRYYDVMANGGTGFCSKHQWNCIDCDSYKP |
| | | | GNTFITVEAA |
| | | | LDLSKELKRPIQPTD/NAAVFYAQSLFRPILMV |
| | | | DKNLITTANTGTSVTETMFDVYVDTFLSMFDVDKK |
| | | | SLNALIATA/ |
| | | | ELTDESCNNLVPTYLKSDNIVAADLGVLIQNSAKH |
| | | | VQGNVAKIAG |
| | | | VSCIWSVDAF |
| Short | OC43- | aa2875-2902 | SADGVQCYTPHSQISYSNFYASGCVLSS |
| peptide | nsp4-S1 | | |
| | OC43- | aa2893-2919 | FYASGCVLSSACTMFMADGSPQPYCY/ |
| | nsp4-S2 | | |
| | OC43- | aa2932-2957 | SLVPHVRYNLANAKGFIRFPEVLREG |
| | nsp4-S3 | | |
| | OC43- | aa2948-2973 | IRFPEVLREGLVRIVRTRSMSYCRVG |
| | nsp4-S4 | | |
| | OC43- | aa2964-2988 | TRSMSYCRVGLCEEADEGICFNFNG |
| | nsp4-S5 | | |
| | OC43- | aa2979-3003 | DEGICFNFNGSWVLNNDYYRSLPGT |
| | nsp4-S6 | | |
| | OC43- | aa2994-3018 | NDYYRSLPGTFCGRDVFDLIYQLFK |
| | nsp4-S7 | | |
| | OC43- | aa3009-3033 | VFDLIYQLFKGLAQPVDFLALTASS |
| | nsp4-S8 | | |
| | OC43- | aa3024-3048 | VDFLALTASSIAGAILAVIVVLVFY |
| | nsp4-S9 | | |
| | OC43- | aa3039-3064 | LAVIVVLVFYYLIKLKRAFGDYTSVV/ |
| | nsp4-S10 | | |
| | OC43- | aa3187-3216 | NRYLSLYNKYRYYSGKMDTAAYREAACSQL |
| | nsp4-S11 | | |
| | OC43- | aa3207-3236 | AYREAACSQLAKAMDTFTNNNGSDVLYQPP |
| | nsp4-S12 | | |
| Extended | OC43- | aa2875-2919 | SADGVQCYTPHSQISYSNFYASGCVLSSACTMFTMADGSP |
| peptide | nsp4-E1 | | QPYCY |
| | OC43- | aa2932-2977 | SLVPHVRYNLANAKGFIRFPEVLREGLVRIVRTRSM |
| | nsp4-E2 | | SYCRVGLCEE |
| | OC43- | aa2968-3011 | SYCRVGLCEEADEGICFNFNGSWVLNNDYYRSLPG |
| | nsp4-E3 | | TFCGRDVFD |
| | OC43- | aa3002-3046 | GTFCGRDVFDLIYQLFKGLAQPVDFLALTASSIAGA |
| | nsp4-E4 | | ILAVIVVLV |
| | OC43- | aa3038-3064/ | ILAVIVVLVFYYLIKLKRAFGDYTSVV/ |
| | nsp4-E5 | aa3187-3203 | NRYLSLYNKYRYYSGKM |
| | OC43- | aa3195-3236 | KYRYYSGKMDTAAYREAACSQLAKAMDTFTNNN |
| | nsp4-E6 | | GSDVLYQPP |
| Short | OC43- | aa2875-2919/ | SADGVQCYTPHSQISYSNFYASGCVLSSACTMFTM |
| protein | nsp4-P1 | aa2932-3006 | ADGSPQPYCY/ |
| | | | SLVPHVRYNLANAKGFIRFPEVLREGLVRIVRTRSM |
| | | | SYCRVGLCEEA |
| | | | DEGICFNFNGSWVLNNDYYRSLPGTFCG |
| | OC43- | aa2996-3064/ | YYRSLPGTFCGRDVFDLIYQLFKGLAQPVDFLALTA |
| | nsp4-P2 | aa3187-3236 | SSIAGAILA |
| | | | VIVVLVFYYLIKLKRAFGDYTSVV/ |
| | | | NRYLSLYNKYRYYSGKMDTAAYREAACSQLAKA |
| | | | MDTFTNNN |
| | | | GSDVLYQPP |
| Full | OC43- | aa2875-2919/ | SADGVQCYTPHSQISYSNFYASGCVLSSACTMFTM |
| length | nsp4-FL | aa2932-3064/ | ADGSPQPYCY/ |
| | | aa3187-3236 | SLVPHVRYNLANAKGFIRFPEVLREGLVRIVRTRSM |
| | | | SYCRVGLCEEA |
| | | | DEGIC |
| | | | FNFNGSWVLNNDYYRSLPGTFCGRDVFDLIYQLFK |
| | | | GLAQPVDFLAL |
| | | | TASSIA |
| | | | GAILAVIVVLVFYYLIKLKRAFGDYTSVV/ |
| | | | NRYLSLYNKYRYYSGKMDTAAYREAACSQLAKA |
| | | | MDTFTNNN |
| | | | GSDVLYQPP |

¹aa positions based on OC43 reference ORF1ab protein sequence, YP_009555238.1
²For conjugation to polyionic VLPS, the peptide/protein antigens have a N-terminal TAG and AAYY proteolytic processing sequence.
/ indicates break in native amino acid sequence

TABLE 15
OC43 antigens of nonstructural proteins nsp6 and nsp7

| Immunogen | Seq ID | aa positions¹ | Amino acid sequence² |
|---|---|---|---|
| Short | OC43-nsp6-S1 | aa3602-3622 | SLAMLLVKHKHLYLTMYITPV |
| peptide | | | |
| | OC43-nsp6-S2 | aa3613-3633 | LYLTMYITPVLFTLLYNNYLV |
| | OC43-nsp6-S3 | aa3753-3780 | IKIVLLCYLFIGYIISCYWGLFSLMNSL |
| | OC43-nsp6-S4 | aa3770-3798 | WGLFSLMNSLFRMPLGVYNYKISVQELRY |
| | OC43-nsp6-S5 | aa3789-3816 | YKISVQELRYMNANGLRPPKNSFEALML |
| | OC43-nsp6-S6 | aa3807-3836 | PKNSFEALMLNFKLLGIGGVPIIEVSQFQ |
| Extended | OC43-nsp6-E1 | aa3602-3633 | SLAMLLVKHKHLYLTMYITPVLFTLLYNNY |
| peptide | | | LV |
| | OC43-nsp6-E2 | aa3753-3799 | IKIVLLCYLFIGYIISCYWGLFSLMNSLFRMPL |
| | | | GVYNYKISVQELRY |
| | OC43-nsp6-E3 | aa3790-3836 | YKISVQELRYMNANGLRPPKNSFEALMLNF |
| | | | KLLGIGGVPIIEVSQFQ |
| Short | OC43-nsp6-P1 | aa3602-3633/ | SLAMLLVKHKHLYLTMYITPVLFTLLYNNY |
| protein | | aa3753-3836 | LV/ |
| | | | IKIVLLCYLFIGYIISCYWGLFSLMNSLFRMPL |
| | | | GVYNYKISVQELRYMNANGLRPPKNSFEAL |
| | | | MLNFKLLGIGGVPIIEVSQFQ |
| Short | OC43-nsp7-S1 | aa3837-3862 | SKLTDVKCANVVLLNCLQHLHVASNS |
| peptide | | | |
| | OC43-nsp7-S2 | aa3853-3878 | LQHLHVASNSKLWHYCSTLHNEILAT |
| | OC43-nsp7-S3 | aa3869-3894 | STLHNEILATSDLSVAFEKLAQLLIV |
| | OC43-nsp7-S4 | aa3885-3910 | FEKLAQLLIVLFANPAAVDSKCLTSI |
| | OC43-nsp7-S5 | aa3901-3925 | AVDSKCLTSIEEVCDDYAKDNTVLQ |
| Extended | OC43-nsp-7- | aa3837-3875 | SKLTDVKCANVVLLNCLQHLHVASNSKLW |
| peptide | E1 | | HYCSTLHNEI |
| | OC43-nsp-7- | aa3866-3903 | HYCSTLHNEILATSDLSVAFEKLAQLLIVLF |
| | E2 | | ANPAAVD |
| Short | OC43-nsp7-P1 | aa3837-3925 | SKLTDVKCANVVLLNCLQHLHVASNSKLW |
| protein | | | HYCSTLHNEILATSDLSVAFEKLAQLLIVLF |
| | | | ANPAAVDSKCLTSIEEVCDDYAKDNTVLQ |

¹aa positions based on OC43 reference ORF1ab protein sequence, YP_009555238.1
²For conjugation to polyionic VLPS, the peptide/protein antigens have a N-terminal TAG and AAYY proteolytic processing sequence.
/ indicates break in native amino acid sequence

TABLE 16
OC43 antigens homologous to the nsp12-1 nonstructural
protein fragment of SARS-CoV-2

| Immunogen | Seq ID | aa-position¹ | Amino acid sequence² |
|---|---|---|---|
| Short | OC43 | aa4525-4555 | YFTKKDWYDFVENPDIINVYKKLGPIFNRAL |
| peptide | nsp12-1-S1 | | |
| | OC43 | aa4546-4575 | KLGPIFNRALVSATEFADKLVEVGLVGVLT |
| | nsp12-1-S2 | | |
| | OC43 | aa4566-4594 | VEVGLVGVLTLDNQDLNGKWYDFGDYVIA |
| | nsp12-1-S3 | | |
| | OC43 | aa4585-4615 | WYDFGDYVIAAPGCGVAIADSYYSYIMPMLT |
| | nsp12-1-S4 | | |
| | SARS- | aa4634-4663 | DLVQYDFTDYKLELFNKYFKHWSMPYHPNT |
| | CoV-2 | | |
| | nsp12-1-S5 | | |
| | OC43 | aa4654-4683 | HWSMPYHPNTVDCQDDRCIIHCANFNILFS |
| | nsp12-1-S6 | | |
| | OC43 | aa4674-4703 | HCANFNILFSMVLPNTCFGPLVRQIFVDG |
| | nsp12-1-S7 | | |
| | OC43 | aa4693-4721 | PLVRQIFVDGVPFVVSIGYHYKELGIVMN |
| | nsp12-1-S8 | | |
| | OC43 | aa4712-4740 | HYKELGIVMNMDVDTHRYRLSLKDLLLYA |
| | nsp12-1-S9 | | |
| Extended | OC43 | aa4525-4574 | YFTKKDWYDFVENPDIINVYKKLGPIFNRALVSATEFAD |
| peptide | nsp12-1-E1 | | KLVEVGLVGVL |
| | OC43 | aa4565-4615 | LVEVGLVGVLTLDNQDLNGKWYDFGDYVIAAPGCGVAIA |
| | nsp12-1-E2 | | DSYYSYIMPMLT |
| | OC43 | aa4634-4675 | DLVQYDFTDYKLELFNKYFKHWSMPYHPNTVDCQDDRCI |
| | nsp12-1-E3 | | IHC |
| | OC43 | aa4666-4707 | CQDDRCIIHCANFNILFSMVLPNTCFGPLVRQIFVDGVP |
| | nsp12-1-E4 | | FVV |
| | OC43 | aa4698-4740 | IFVDGVPFVVSIGYHYKELGIVMNMDVDTHRYRLSLKDL |
| | nsp12-1-E5 | | LLYA |
| Short | OC43 | aa4525-4615 | YFTKKDWYDFVENPDIINVYKKLGPIFNRALVSATEFAD |
| protein | nsp12-1- | | KLVEVGLVGVLTLDNQDLNGKWYDFGDYVIAAPGCGVAI |
| | SP1 | | ADSYYSYIMPMLT |
| | OC43 | aa4634-4740 | DLVQYDFTDYKLELFNKYFKHWSMPYHPNTVDCQDDRCI |
| | nsp12-1- | | IHCANFNILFSMVLPNTCFGPLVRQIFVDGVPFVVSIGY |
| | SP2 | | HYKELGIVMNMDVDTHRYRLSLKDLLLYA |
| Full | OC43 | aa4525- | YFTKKDWYDFVENPDIINVYKKLGPIFNRALVSATEFAD |
| length | nsp12-1-FL | 4615/ | KLVEVGLVGVLTLDNQDLNGKWYDFGDYVIAAPGCGVAI |
| | | aa4634- | ADSYYSYIMPMLT/DLVQYDFTDYKLELFNKYFKHWSMP |
| | | 4740 | YHPNTVDCQDDRCIIHCANFNILFSMVLPNTCFGPLVRQ |
| | | | IFVDGVPFVVSIGYHYKELGIVMNMDVDTHRYRLSLKDL |
| | | | LLYA |

^{l}aa positions based on OC43 reference ORF1ab protein (YP_009555238.1).
²For conjugation to polyionic VLPS, the peptide/protein antigens have a N-terminal TAG and AAYY proteolytic processing sequence.
/ indicates break in native amino acid sequence

TABLE 17
OC43 antigens homologous to of the nsp12-2 nonstructural
protein fragment of SARS-CoV-2

| Immunogen | Seq ID | aa-position | Amino acid sequence |
|---|---|---|---|
| Short | OC43 | aa4894-4921 | AYTKRNVLPTLTQMNLKYAISAKNRART |
| peptide | nsp12-2- | | |
| | S1 | | |
| | OC43 | aa4912-4939 | AISAKNRARTVAGVSILSTMTGRMFHQK |
| | nsp12-2- | | |
| | S2 | | |
| | OC43 | aa4930-4957 | TMTGRMFHQKCLKSIAATRGVPVVIGTT |
| | nsp12-2- | | |
| | S3 | | |
| | OC43 | aa4948-4975 | RGVPVVIGTTKFYGGWDDMLRRLIKDVD |
| | nsp12-2- | | |
| | S4 | | |
| | OC43 | aa4966-4993 | MLRRLIKDVDNPVLMGWDYPKCDRAMPN |
| | nsp12-2- | | |
| | S5 | | |
| | OC43 | aa4984-5012 | YPKCDRAMPNLLRIVSSLVLARKHETCC |
| | nsp12-2- | | S |
| | S6 | | |
| | OC43 | aa5003-5030 | LARKHETCCSQSDRFYRLANECAQVLSE |
| | nsp12-2- | | |
| | S7 | | |
| | OC43 | aa5021-5048 | ANECAQVLSEIVMCGGCYYVKPGGTSSG |
| | nsp12-2- | | |
| | S8 | | |
| | OC43 | aa5039-5067 | YVKPGGTSSGDATTAFANSVFNICQAVS |
| | nsp12-2- | | A |
| | S9 | | |
| | OC43 | aa5058-5085 | VFNICQAVSANVCALMSCNGNKIEDLSI |
| | nsp12-2- | | |
| | S10 | | |
| | OC43 | aa5076-5103 | NGNKIEDLSIRALQKRLYSHVYRSDKVD |
| | nsp12-2- | | |
| | S11 | | |
| | OC43 | aa5094-5121 | SHVYRSDKVDSTFVTEYYEFLNKHFSMM |
| | nsp12-2- | | |
| | S12 | | |
| | OC43 | aa5112-5140 | EFLNKHFSMMILSDDGVVCYNSDYASKG |
| | nsp12-2- | | Y |
| | S13 | | |
| | OC43 | aa5131-5159 | YNSDYASKGYIANISAFQQVLYYQNNVF |
| | nsp12-2- | | M |
| | S14 | | |
| | OC43 | aa5150-5177 | VLYYQNNVFMSESKCWVEHDINNGPHEF |
| | nsp12-2- | | |
| | S15 | | |
| | OC43 | aa5168-5196 | HDINNGPHEFCSQHTMLVKMDGDDVYLP |
| | nsp12-2- | | Y |
| | S16 | | |
| | OC43 | aa5187-5215 | MDGDDVYLPYPNPSRILGAGCFVDDLLK |
| | nsp12-2- | | T |
| | S17 | | |
| | OC43 | aa5206-5234 | GCFVDDLLKTDSVLLIERFVSLAIDAYP |
| | nsp12-2- | | L |
| | S18 | | |
| Extended | OC43 | aa4894-4940 | AYTKRNVLPTLTQMNLKYAISAKNRART |
| peptide | nsp12-2- | | VAGVSILSTMTGRMFHQKC |
| | E1 | | |
| | OC43 | aa4931-4977 | MTGRMFHQKCLKSIAATRGVPVVIGTTK |
| | nsp12-2- | | FYGGWDDMLRRLIKDVDNP |
| | E2 | | |
| | OC43 | aa4968-5013 | RRLIKDVDNPVLMGWDYPKCDRAMPN |
| | nsp12-2- | | LLRIVSSLVLARKHETCCSQ |
| | E3 | | |
| | OC43 | aa5004-5049 | ARKHETCCSQSDRFYRLANECAQVLSEI |
| | nsp12-2- | | VMCGGCYYVKPGGTSSGD |
| | E4 | | |
| | OC43 | aa5040-5085 | VKPGGTSSGDATTAFANSVFNICQAVSA |
| | nsp12-2- | | NVCALMSCNGNKIEDLSI |
| | E5 | | |
| | OC43 | aa5076-5121 | NGNKIEDLSIRALQKRLYSHVYRSDKVDS |
| | nsp12-2- | | TFVTEYYEFLNKHFSMM |
| | E6 | | |
| | OC43 | aa5112-5158 | EFLNKHFSMMILSDDGVVCYNSDYASKG |
| | nsp12-2- | | YIANISAFQQVLYYQNNVF |
| | E7 | | |
| | OC43 | aa5149-5195 | QVLYYQNNVFMSESKCWVEHDINNGP |
| | nsp12-2- | | HEFCSQHTMLVKMDGDDVYLP |
| | E8 | | |
| | OC43 | aa5188-5234 | KMDGDDVYLPYPNPSRILGAGCFVDDLL |
| | nsp12-2- | | KTDSVLLIERFVSLAIDAYPL |
| | E9 | | |
| Short | OC43 | aa4894-5018 | AYTKRNVLPTLTQMNLKYAISAKNRART |
| protein | nsp12-2- | | VAGVSILSTMTGRMFHQKCLKSIAATRG |
| | SP1 | | VPVVIGTTKFYGGWDDMLRRLIKDVDN |
| | | | PVLMGWDYPKCDRAMPNLLRIVSSLVL |
| | | | ARKHETCCSQSDRFY |
| | OC43 | aa5008-5132 | ETCCSQSDRFYRLANECAQVLSEIVMCG |
| | nsp12-2- | | GCYYVKPGGTSSGDATTAFANSVFNICQ |
| | SP2 | | AVSANVCALMSCNGNKIEDLSIRALQKR |
| | | | LYSHVYRSDKVDSTFVTEYYEFLNKHFS |
| | | | MMILSDDGVVCYN |
| | OC43 | aa5122-5234 | ILSDDGVVCYNSDYASKGYIANISAFQQV |
| | nsp12-2- | | LYYQNNVFMSESKCWVEHDINNGPHEF |
| | SP3 | | CSQHTMLVKMDGDDVYLPYPNPSRILG |
| | | | AGCFVDDLLKTDSVLLIERFVSLAIDAYP |
| | | | L |
| Full | OC43 | aa4894-5234 | AYTKRNVLPTLTQMNLKYAISAKNRART |
| length | nsp12-2- | | VAGVSILSTMTGRMFHQKCLKSIAATRG |
| antigen | FL | | VPVVIGTTKFYGGWDDMLRRLIKDVDN |
| | | | PVLMGWDYPKCDRAMPNLLRIVSSLVL |
| | | | ARKHETCCSQSDRFYRLANECAQVLSEI |
| | | | VMCGGCYYVKPGGTSSGDATTAFANSV |
| | | | FNICQAVSANVCALMSCNGNKIEDLSIR |
| | | | ALQKRLYSHVYRSDKVDSTFVTEYYEFL |
| | | | NKHFSMMILSDDGVVCYNSDYASKGYIA |
| | | | NISAFQQVLYYQNNVFMSESKCWVEHDI |
| | | | NNGPHEFCSQHTMLVKMDGDDVYLPY |
| | | | PNPSRILGAGCFVDDLLKTDSVLLIERFVS |
| | | | LAIDAYPL |

¹aa positions based on OC43 reference ORF1ab protein (YP_009555238.1).
²For conjugation to polyionic VLPS, the peptide/protein antigens have a N-terminal TAG and AAYY proteolytic processing sequence.

### REFERENCE LIST

Braun, J., Loyal, L., Frentsch, M., Wendisch, D., Georg, P., Kurth, F., Hippenstiel, S., Dingeldey, M., Kruse, B., Fauchere, F., Baysal, E., Mangold, M., Henze, L., Lauster, R., Mall, M. A., Beyer, K., Rohmel, J., Voigt, S., Schmitz, J., Miltenyi, S., Demuth, I., Muller, M. A., Hocke, A., Witzenrath, M., Suttorp, N., Kern, F., Reimer, U., Wenschuh, H., Drosten, C., Corman, V. M., Giesecke-Thiel, C., Sander, L. E., and Thiel, A. (2020). SARS-CoV-2-reactive T cells in healthy donors and patients with COVID-19. Nature.
Chan, J., Zhang, A. J., Yuan, S., Poon, V., Chan, C., Lee, A., Chan, W. M., Fan, Z., Tsoi, H-W., Wen, L., Liang, R., Cao, J., Chen, Y., Tang, K., Luo, C., Cai, J-P., Kok, K-H., Chu, H., Chan, K-H., Sridhar, S., Chen, Z. Chen, H., To, K. K-W., Yuen, K-Y. (2020). Simulation of the Clinical and Pathological Manifestations of Coronavirus Disease 2019 (COVID-19) in a Golden Syrian Hamster Model: Implications for Disease Pathogenesis and Transmissibility. Clin Infect Dis. 71:2428-2446.
Chen, Y., Liu, Q., and Guo, D. (2020). Emerging coronaviruses: Genome structure, replication, and pathogenesis. J Med. Virol.
Grifoni, A., Weiskopf, D., Ramirez, S. I., Mateus, J., Dan, J. M., Moderbacher, C. R., Rawlings, S. A., Sutherland, A., Premkumar, L., Jadi, R. S., Marrama, D., de Silva, A. M., Frazier, A., Carlin, A. F., Greenbaum, J. A., Peters, B., Krammer, F., Smith, D. M., Crotty, S., and Sette, A. (2020). Targets of T Cell Responses to SARS-CoV-2 Coronavirus in Humans with COVID-19 Disease and Unexposed Individuals. Cell 181, 1489-1501.
Grifoni, A., Sidney, J., Vita, R., Peters, B., Crotty, S., Weiskopt, D., and Sette, A. (2021). SARS-CoV-2 human T cell epitopes: Adaptive immune response against COVID-19. Cell Host & Microbe 29, 1076-1092.
Kersh, G. J. and Allen, P. M. (1996). Structural basis for T cell recognition of altered peptide ligands: a single T cell receptor can productively recognize a large continuum of related ligands. J Exp Med. 184, 1259-1268.
Le, B. N., Tan, A. T., Kunasegaran, K., Tham, C. Y. L., Hafezi, M., Chia, A., Chng, M. H. Y., Lin, M., Tan, N., Linster, M., Chia, W. N., Chen, M. I., Wang, L. F., Ooi, E. E., Kalimuddin, S., Tambyah, P. A., Low, J. G., Tan, Y. J., and Bertoletti, A. (2020). SARS-CoV-2-specific T cell immunity in cases of COVID-19 and SARS, and uninfected controls. Nature 584, 457-462.
Liu, W. J., Zhao, M., Liu, K., Xu, K., Wong, G., Tan, W., and Gao, G. F. (2017). T-cell immunity of SARS-COV: Implications for vaccine development against MERS-COV. Antiviral Res. 137, 82-92.
Mason, D. (1998). A very high level of crossreactivity is an essential feature of the T-cell receptor. Immunol. Today 19, 395-404.

Mateus, J., Grifoni, A., Tarke, A., Sidney, J., Ramirez, S. I., Dan, J. M., Burger, Z. C., Rawlings, S. A., Smith, D. M., Phillips, E., Mallal, S., Lammers, M., Rubiro, P., Quiambao, L., Sutherland, A., Yu, E. D., da Silva, A. R., Greenbaum, J., Frazier, A., Markmann, A. J., Premkumar, L., de, S. A., Peters, B., Crotty, S., Sette, A., and Weiskopf, D. (2020). Selective and cross-reactive SARS-CoV-2 T cell epitopes in unexposed humans. Science.
Petrosillo, N., Viceconte, G., Ergonul, O., Ippolito, G., and Petersen, E. (2020). COVID-19, SARS and MERS: are they closely related? Clin Microbiol. Infect. 26, 729-734.
Rosenke, K., Meade-White, K., Letko, M., Clancy, C., Hansen, F., Liu, Y., Okumura, A., Tang-Huau, T L., Li, R., Saturday, G., Feldmann, F., Scott, D., Wang, Z., Munster, V., Jarvis, M. A., F eldmann, H. (2020). Defining the Syrian hamster as a highly susceptible preclinical model for SARS-CoV-2 infection. Emerg Microbes Infect. 9:2673-2684.
Severance, E. G., Bossis, I., Dickerson, F. B., Stallings, C. R., Origoni, A. E., Sullens, A., Yolken, R. H., and Viscidi, R. P. (2008). Development of a nucleocapsid-based human coronavirus immunoassay and estimates of individuals exposed to coronavirus in a U.S. metropolitan population. Clin Vaccine Immunol. 15, 1805-1810.
Sewell, A. K. (2012). Why must T cells be cross-reactive? Nat. Rev. Immunol. 12, 669-677.
Sun, S. H., Chen, Q., Gu, H. J., Yang, G., Wang, Y. X., Huang, X. Y., Liu, S. S., Zhang, N. N., Li, X. F., Xiong, R., Guo, Y., Deng, Y. Q., Huang, W. J., Liu, Q., Liu, Q. M., Shen, Y. L., Zhou, Y., Yang, X., Zhao, T. Y., Fan, C. F., Zhou, Y. S., Qin, C. F., and Wang, Y. C. (2020). A Mouse Model of SARS-CoV-2 Infection and Pathogenesis. Cell Host. Microbe 28, 124-133.
Weiskopf, D., Schmitz, K. S., Raadsen, M. P., Grifoni, A., Okba, N. M. A., Endeman, H., van den Akker, J. P. C., Molenkamp, R., Koopmans, M. P. G., van Gorp, E. C. M., Haagmans, B. L., de Swart, R. L., Sette, A., and de Vries, R. D. (2020). Phenotype and kinetics of SARS-CoV-2-specific T cells in COVID-19 patients with acute respiratory distress syndrome. Sci. Immunol. 5.
Wilson, D. B., Wilson, D. H., Schroder, K., Pinilla, C., Blondelle, S., Houghten, R. A., and Garcia, K. C. (2004). Specificity and degeneracy of T cells. Mol. Immunol. 40, 1047-1055.

1. A composition comprising (a) a chimeric papillomavirus virus-like particle (VLP) comprising an L1 protein, having an amino acid insert inserted into the HI loop of the L1 protein and (b) a coronavirus antigen.

2. A composition according to claim 1 wherein said amino acid insert comprises a contiguous sequence of negatively charged amino acids and a terminal cysteine residue.

3. (canceled)

4. (canceled)

5. A composition according to claim 2, wherein said amino acid insert is selected from the inserts identified in Table 1.

6. A composition according to claim 1, wherein said amino acid insert is inserted into the HI loop of the L1 protein at a location between positions 344 and 357.

7. (canceled)

8. A composition according to claim 6, wherein said amino acid insert replaces the native amino acid sequences identified in Table 1.

9. A composition according to claim 1, wherein said coronavirus antigen is selected from the group consisting of an OC43 antigen, an HKU1 antigen, a 229E antigen, an NL63 antigen, a SARS-CoV-1 antigen, a MERS antigen, a SARS-CoV-2 antigen, and fusions thereof.

10. (canceled)

11. A composition according to claim 9, wherein said coronavirus antigen comprises a viral structural protein selected from the group consisting of the membrane protein (M), the nucleocapsid protein (N), and the S2 region of the spike(S) envelope protein from OC43, HKU1, 229E, NL63, SARS-CoV-1, MERS, and/or SARS-CoV-2 coronaviruses or a viral non-structural protein selected from the group consisting of nsp3, nsp4, nsp6, nsp7, and nsp12 proteins from OC43, HKU1, 229E, NL63, SARS-CoV-1, MERS, and/or SARS-CoV-2 coronaviruses.

12. (canceled)

13. (canceled)

14. (canceled)

15. A composition according to claim 9, wherein said coronavirus antigen is selected from one or more of the amino acid sequences in Tables 3-10.

16. (canceled)

17. (canceled)

18. (canceled)

19. (canceled)

20. (canceled)

21. (canceled)

22. (canceled)

23. A composition according to claim 9, wherein said coronavirus antigen comprises one or more viral proteins of the OC43 coronavirus and/or HKY1 the coronavirus, wherein said one or more viral proteins comprise 70% or greater identity with an amino acid sequence of SARS-CoV-1 coronavirus, MERS coronavirus, and/or SARS-CoV-2 coronavirus.

24. (canceled)

25. A composition according to claim 9, wherein said OC43 and/or HKU1 coronavirus structural protein antigens is selected from one or more of the amino acid sequences in Tables 11-13 and/or said OC43 coronavirus nonstructural antigen is selected from one or more of the amino acid sequences in Tables 14-17.

26. (canceled)

27. (canceled)

28. (canceled)

29. A composition according to claim 9, wherein said coronavirus antigen comprises a first antigen selected from the structural proteins and peptides of OC43 and/or HKU1 coronavirus, a second antigen selected from the non-structural proteins and peptides of OC43, a third antigen selected from the structural proteins and peptides of SARS-CoV-2 coronavirus, and a fourth antigen selected from the nonstructural proteins and peptides of SARS-CoV-2.

30. A composition according to claim 1, further comprising a TAG sequence linked at a first end to said amino acid insert and linked at a second end to said coronavirus antigen.

31. (canceled)

32. (canceled)

33. (canceled)

34. A composition according to claim 30, wherein said TAG sequence comprises an amino acid sequence identified in Table 2.

35. A composition according to claim 1, wherein said composition is effective to stimulate a cytotoxic T cell response in a mammal.

36. A method for stimulating a cytotoxic T cell response to a coronavirus in a mammal or for stimulating both therapeutic and protective immunity to a coronavirus in said mammal comprising administering to said mammal a composition according to any one of claims 1-14.

37. (canceled)

38. (canceled)

39. (canceled)

40. (canceled)

41. (canceled)

42. (canceled)

43. (canceled)

44. (canceled)

45. (canceled)


SEQUENCE LISTING (444 sequences)

SEQ ID NO: 1 - protein, 4 aa, Artificial Sequence
  Description of Artificial Sequence Synthetic peptide
AAYY

SEQ ID NO: 2 - protein, 4 aa, Artificial Sequence
  Description of Artificial Sequence Synthetic peptide
GSSG

SEQ ID NO: 3 - protein, 8 aa, Artificial Sequence
  Description of Artificial Sequence Synthetic peptide
RKHRKHRK

SEQ ID NO: 4 - protein, 14 aa, Artificial Sequence
  Description of Artificial Sequence Synthetic peptide
CRRRRRRRRCAAYY

SEQ ID NO: 5 - protein, 9 aa, Artificial Sequence
  Description of Artificial Sequence Synthetic peptide
EEEEEEEEC

SEQ ID NO: 6 - protein, 8 aa, Artificial Sequence
  Description of Artificial Sequence Synthetic peptide
CEEEEEEE

SEQ ID NO: 7 - protein, 9 aa, Artificial Sequence
  Description of Artificial Sequence Synthetic peptide
DDDDDDDDC

SEQ ID NO: 8 - protein, 9 aa, Artificial Sequence
  Description of Artificial Sequence Synthetic peptide
EDEDEDEDC

SEQ ID NO: 9 - protein, 5 aa, Artificial Sequence
  Description of Artificial Sequence Synthetic peptide
EEEEC

SEQ ID NO: 10 - protein, 6 aa, Artificial Sequence
  Description of Artificial Sequence Synthetic peptide
EEEEEC

SEQ ID NO: 11 - protein, 7 aa, Artificial Sequence
  Description of Artificial Sequence Synthetic peptide
EEEEEEC

SEQ ID NO: 12 - protein, 8 aa, Artificial Sequence
  Description of Artificial Sequence Synthetic peptide
EEEEEEEC

SEQ ID NO: 13 - protein, 10 aa, Artificial Sequence
  Description of Artificial Sequence Synthetic peptide
EEEEEEEEEC

SEQ ID NO: 14 - protein, 11 aa, Artificial Sequence
  Description of Artificial Sequence Synthetic peptide
EEEEEEEEEEC

SEQ ID NO: 15 - protein, 10 aa, Artificial Sequence
  Description of Artificial Sequence Synthetic peptide
CEEEEEEEEC

SEQ ID NO: 16 - protein, 17 aa, Artificial Sequence
  Description of Artificial Sequence Synthetic peptide
GSSGEEEEEEEECGSSG

SEQ ID NO: 17 - protein, 9 aa, Severe acute respiratory syndrome coronavirus 2
GTPLTEYDS

SEQ ID NO: 18 - protein, 5 aa, Severe acute respiratory syndrome coronavirus 2
GTPLT

SEQ ID NO: 19 - protein, 6 aa, Severe acute respiratory syndrome coronavirus 2
GTPLTE

SEQ ID NO: 20 - protein, 7 aa, Severe acute respiratory syndrome coronavirus 2
GTPLTEY

SEQ ID NO: 21 - protein, 8 aa, Severe acute respiratory syndrome coronavirus 2
GTPLTEYD

SEQ ID NO: 22 - protein, 10 aa, Severe acute respiratory syndrome coronavirus 2
DGTPLTEYDS

SEQ ID NO: 23 - protein, 11 aa, Severe acute respiratory syndrome coronavirus 2
DGTPLTEYDSS

SEQ ID NO: 24 - protein, 7 aa, Severe acute respiratory syndrome coronavirus 2
TPLTEYD

SEQ ID NO: 25 - protein, 5 aa, Severe acute respiratory syndrome coronavirus 2
TPLTE

SEQ ID NO: 26 - protein, 9 aa, Artificial Sequence
  Description of Artificial Sequence Synthetic peptide
RRRRRRRRC

SEQ ID NO: 27 - protein, 9 aa, Artificial Sequence
  Description of Artificial Sequence Synthetic peptide
KKKKKKKKC

SEQ ID NO: 28 - protein, 9 aa, Artificial Sequence
  Description of Artificial Sequence Synthetic peptide
HHHHHHHHC

SEQ ID NO: 29 - protein, 9 aa, Artificial Sequence
  Description of Artificial Sequence Synthetic peptide
RKHRKHRKC

SEQ ID NO: 30 - protein, 5 aa, Artificial Sequence
  Description of Artificial Sequence Synthetic peptide
RRRRC

SEQ ID NO: 31 - protein, 6 aa, Artificial Sequence
  Description of Artificial Sequence Synthetic peptide
RRRRRC

SEQ ID NO: 32 - protein, 7 aa, Artificial Sequence
  Description of Artificial Sequence Synthetic peptide
RRRRRRC

SEQ ID NO: 33 - protein, 8 aa, Artificial Sequence
  Description of Artificial Sequence Synthetic peptide
RRRRRRRC

SEQ ID NO: 34 - protein, 10 aa, Artificial Sequence
  Description of Artificial Sequence Synthetic peptide
RRRRRRRRRC

SEQ ID NO: 35 - protein, 11 aa, Artificial Sequence
  Description of Artificial Sequence Synthetic peptide
RRRRRRRRRRC

SEQ ID NO: 36 - protein, 9 aa, Artificial Sequence
  Description of Artificial Sequence Synthetic peptide
CRRRRRRRR

SEQ ID NO: 37 - protein, 10 aa, Artificial Sequence
  Description of Artificial Sequence Synthetic peptide
CRRRRRRRRC

SEQ ID NO: 38 - protein, 31 aa, Severe acute respiratory syndrome coronavirus 2
GTITVEELKKLLEQWNLVIGFLFLTWICLLQ

SEQ ID NO: 39 - protein, 31 aa, Severe acute respiratory syndrome coronavirus 2
LFLTWICLLQFAYANRNRFLYIIKLIFLYWL

SEQ ID NO: 40 - protein, 30 aa, Severe acute respiratory syndrome coronavirus 2
IIKLIFLWLLWPVTLACFVLAAVYRINWIT

SEQ ID NO: 41 - protein, 30 aa, Severe acute respiratory syndrome coronavirus 2
AAVYRINWITGGIAIAMACLVGLMWLSYFI

SEQ ID NO: 42 - protein, 30 aa, Severe acute respiratory syndrome coronavirus 2
VGLMWLSYFIASFRLFARTRSMWSFNPETN

SEQ ID NO: 43 - protein, 31 aa, Severe acute respiratory syndrome coronavirus 2
SMWSFNPETNILLNVPLHGTILTRPLLESEL

SEQ ID NO: 44 - protein, 31 aa, Severe acute respiratory syndrome coronavirus 2
LTRPLLESELVIGAVILRGHLRIAGHHLGRC

SEQ ID NO: 45 - protein, 30 aa, Severe acute respiratory syndrome coronavirus 2
RIAGHHLGRCDIKDLPKEITVATSRTLSYY

SEQ ID NO: 46 - protein, 31 aa, Severe acute respiratory syndrome coronavirus 2
VATSRTLSYYKLGASQRVAGDSGFAAYSRYR

SEQ ID NO: 47 - protein, 31 aa, Severe acute respiratory syndrome coronavirus 2
SGFAAYSRYRIGNYKLNTDHSSSSDNIALLV

SEQ ID NO: 48 - protein, 45 aa, Severe acute respiratory syndrome coronavirus 2
GTITVEELKKLLEQWNLVIGFLFLTWICLLQFAYANRNRFLYIIK

SEQ ID NO: 49 - protein, 45 aa, Severe acute respiratory syndrome coronavirus 2
NRNRFLYIIKLIFLWLLWPVTLACFVLAAVYRINWITGGIAIAMA

SEQ ID NO: 50 - protein, 45 aa, Severe acute respiratory syndrome coronavirus 2
ITGGIAIAMACLVGLMWLSYFIASFRLFARTRSMWSFNPETNILL

SEQ ID NO: 51 - protein, 45 aa, Severe acute respiratory syndrome coronavirus 2
SFNPETNILLNVPLHGTILTRPLLESELVIGAVILRGHLRIAGHH

SEQ ID NO: 52 - protein, 45 aa, Severe acute respiratory syndrome coronavirus 2
RGHLRIAGHHLGRCDIKDLPKEITVATSRTLSYYKLGASQRVAGD

SEQ ID NO: 53 - protein, 41 aa, Severe acute respiratory syndrome coronavirus 2
LGASQRVAGDSGFAAYSRYRIGNYKLNTDHSSSSDNIALLV

SEQ ID NO: 54 - protein, 111 aa, Severe acute respiratory syndrome coronavirus 2
GTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPET

SEQ ID NO: 55 - protein, 116 aa, Severe acute respiratory syndrome coronavirus 2
TRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLV

SEQ ID NO: 56 - protein, 216 aa, Severe acute respiratory syndrome coronavirus 2
GTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLV

SEQ ID NO: 57 - protein, 34 aa, Severe acute respiratory syndrome coronavirus 2
SWFTALTQHGKEDLKFPRGQGVPINTNSSPDDQI

SEQ ID NO: 58 - protein, 34 aa, Severe acute respiratory syndrome coronavirus 2
NTNSSPDDQIGYYRRATRRIRGGDGKMKDLSPRW

SEQ ID NO: 59 - protein, 34 aa, Severe acute respiratory syndrome coronavirus 2
GKMKDLSPRWYFYYLGTGPEAGLPYGANKDGIIW

SEQ ID NO: 60 - protein, 34 aa, Severe acute respiratory syndrome coronavirus 2
YGANKDGIIWVATEGALNTPKDHIGTRNPANNAA

SEQ ID NO: 61 - protein, 34 aa, Severe acute respiratory syndrome coronavirus 2
GTRNPANNAAIVLQLPQGTTLPKGFYAEGSRGGS

SEQ ID NO: 62 - protein, 34 aa, Severe acute respiratory syndrome coronavirus 2
FYAEGSRGGSQASSRSSSRSRNSSRNSTPGSSRG

SEQ ID NO: 63 - protein, 34 aa, Severe acute respiratory syndrome coronavirus 2
RNSTPGSSRGTSPARMAGNGGDAALALLLLDRLN

SEQ ID NO: 64 - protein, 34 aa, Severe acute respiratory syndrome coronavirus 2
LALLLLDRLNQLESKMSGKGQQQQGQTVTKKSAA

SEQ ID NO: 65 - protein, 34 aa, Severe acute respiratory syndrome coronavirus 2
GQTVTKKSAAEASKKPRQKRTATKAYNVTQAFGR

SEQ ID NO: 66 - protein, 34 aa, Severe acute respiratory syndrome coronavirus 2
AYNVTQAFGRRGPEQTQGNFGDQELIRQGTDYKH

SEQ ID NO: 67 - protein, 34 aa, Severe acute respiratory syndrome coronavirus 2
LIRQGTDYKHWPQIAQFAPSASAFFGMSRIGMEV

SEQ ID NO: 68 - protein, 33 aa, Severe acute respiratory syndrome coronavirus 2
FGMSRIGMEVTPSGTWLTYTGAIKLDDKDPNFK

SEQ ID NO: 69 - protein, 32 aa, Severe acute respiratory syndrome coronavirus 2
KLDDKDPNFKDQVILLNKHIDAYKTFPPTEPK

SEQ ID NO: 70 - protein, 54 aa, Severe acute respiratory syndrome coronavirus 2
SWFTALTQHGKEDLKFPRGQGVPINTNSSPDDQIGYYRRATRRIRGGDGKMKDL

SEQ ID NO: 71 - protein, 54 aa, Severe acute respiratory syndrome coronavirus 2
RGGDGKMKDLSPRWYFYYLGTGPEAGLPYGANKDGIIWVATEGALNTPKDHIGT

SEQ ID NO: 72 - protein, 54 aa, Severe acute respiratory syndrome coronavirus 2
LNTPKDHIGTRNPANNAAIVLQLPQGTTLPKGFYAEGSRGGSQASSRSSSRSRN

SEQ ID NO: 73 - protein, 53 aa, Severe acute respiratory syndrome coronavirus 2
SSRSSSRSRNSSRNSTPGSSRGTSPARMAGNGGDAALALLLLDRLNQLESKMS

SEQ ID NO: 74 - protein, 54 aa, Severe acute respiratory syndrome coronavirus 2
LNQLESKMSGKGQQQQGQTVTKKSAAEASKKPRQKRTATKAYNVTQAFGRRGPE

SEQ ID NO: 75 - protein, 55 aa, Severe acute respiratory syndrome coronavirus 2
TQAFGRRGPEQTQGNFGDQELIRQGTDYKHWPQIAQFAPSASAFFGMSRIGMEVT

SEQ ID NO: 76 - protein, 54 aa, Severe acute respiratory syndrome coronavirus 2
GMSRIGMEVTPSGTWLTYTGAIKLDDKDPNFKDQVILLNKHIDAYKTFPPTEPK

SEQ ID NO: 77 - protein, 113 aa, Severe acute respiratory syndrome coronavirus 2
SWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPG

SEQ ID NO: 78 - protein, 113 aa, Severe acute respiratory syndrome coronavirus 2
NNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTAT

SEQ ID NO: 79 - protein, 115 aa, Severe acute respiratory syndrome coronavirus 2
SKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPK

SEQ ID NO: 80 - protein, 319 aa, Severe acute respiratory syndrome coronavirus 2
SWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPK

SEQ ID NO: 81 - protein, 55 aa, Severe acute respiratory syndrome coronavirus 2
CASYQTQTNSPRRARSVASQSIIAYTMSLGAENSVAYSNNSIAIPTNFTISVTTE

SEQ ID NO: 82 - protein, 54 aa, Severe acute respiratory syndrome coronavirus 2
TNFTISVTTEILPVSMTKTSVDCTMYICGDSTECSNLLLQYGSFCTQLNRALTG

SEQ ID NO: 83 - protein, 55 aa, Severe acute respiratory syndrome coronavirus 2
CTQLNRALTGIAVEQDKNTQEVFAQVKQIYKTPPIKDFGGFNFSQILPDPSKPSK

SEQ ID NO: 84 - protein, 56 aa, Severe acute respiratory syndrome coronavirus 2
ILPDPSKPSKRSFIEDLLFNKVTLADAGFIKQYGDCLGDIAARDLICAQKFNGLTV

SEQ ID NO: 85 - protein, 55 aa, Severe acute respiratory syndrome coronavirus 2
CAQKFNGLTVLPPLLTDEMIAQYTSALLAGTITSGWTFGAGAALQIPFAMQMAYR

SEQ ID NO: 86 - protein, 55 aa, Severe acute respiratory syndrome coronavirus 2
PFAMQMAYRFNGIGVTQNVLYENQKLIANQFNSAIGKIQDSLSSTASALGKLQDV

SEQ ID NO: 87 - protein, 55 aa, Severe acute respiratory syndrome coronavirus 2
ASALGKLQDVVNQNAQALNTLVKQLSSNFGAISSVLNDILSRLDKVEAEVQIDRL

SEQ ID NO: 88 - protein, 55 aa, Severe acute respiratory syndrome coronavirus 2
VEAEVQIDRLITGRLQSLQTYVTQQLIRAAEIRASANLAATKMSECVLGQSKRVD

SEQ ID NO: 89 - protein, 56 aa, Severe acute respiratory syndrome coronavirus 2
CVLGQSKRVDFCGKGYHLMSFPQSAPHGVVFLHVTYVPAQEKNFTTAPAICHDGKA

SEQ ID NO: 90 - protein, 55 aa, Severe acute respiratory syndrome coronavirus 2
APAICHDGKAHFPREGVFVSNGTHWFVTQRNFYEPQIITTDNTFVSGNCDVVIGI

SEQ ID NO: 91 - protein, 55 aa, Severe acute respiratory syndrome coronavirus 2
SGNCDVVIGIVNNTVYDPLQPELDSFKEELDKYFKNHTSPDVDLGDISGINASVV

SEQ ID NO: 92 - protein, 55 aa, Severe acute respiratory syndrome coronavirus 2
DISGINASVVNIQKEIDRLNEVAKNLNESLIDLQELGKYEQYIKWPWYIWLGFIA

SEQ ID NO: 93 - protein, 56 aa, Severe acute respiratory syndrome coronavirus 2
PWYIWLGFIAGLIAIVMVTIMLCCMTSCCSCLKGCCSCGSCCKFDEDDSEPVLKGV

SEQ ID NO: 94 - protein, 128 aa, Severe acute respiratory syndrome coronavirus 2
CASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFG

SEQ ID NO: 95 - protein, 129 aa, Severe acute respiratory syndrome coronavirus 2
IYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVL

SEQ ID NO: 96 - protein, 128 aa, Severe acute respiratory syndrome coronavirus 2
FNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECV

SEQ ID NO: 97 - protein, 130 aa, Severe acute respiratory syndrome coronavirus 2
NLAATKMSECVLGQSKRVDFCGKGYHLMSFPQAPHGVVFLHVTYVPAQEKNFTTAPAICHDGKAHFPREGVFVSNGTHWFVTQRNFYEPQIITTDNTFVSGNCDVVIGIVNNTVYDPLQPELIDSFKEEL

SEQ ID NO: 98 - protein, 127 aa, Severe acute respiratory syndrome coronavirus 2
QPELIDSFKEELDKYFKNHTSPDVDLGDISGINASVVNIQKEDRLNEVAKNLNESLIDLQELGKYEQYIKWPWYIWLGFIAGLIAIVMVTIMLCCMTSCCSCLKGCCSCGSCCKFDEDDSEPVLKGV

SEQ ID NO: 99 - protein, 28 aa, Severe acute respiratory syndrome coronavirus 2
FLYLYALVYFLQSINFVRIIMRLWLCWK

SEQ ID NO: 100 - protein, 25 aa, Severe acute respiratory syndrome coronavirus 2
IIMRLWLCWKCRSKNPLLYDANYFL

SEQ ID NO: 101 - protein, 28 aa, Severe acute respiratory syndrome coronavirus 2
PLLYDANYFLCWHTNCYDYCIPYNSVTS

SEQ ID NO: 102 - protein, 28 aa, Severe acute respiratory syndrome coronavirus 2
YCIPYNSVTSSIVITSGDGTTSPISEHD

SEQ ID NO: 103 - protein, 28 aa, Severe acute respiratory syndrome coronavirus 2
GTTSPISEHDYQIGGYTEKWESGVKDCV

SEQ ID NO: 104 - protein, 28 aa, Severe acute respiratory syndrome coronavirus 2
KWESGVKDCVVLHSYFTSDYYQLYSTQL

SEQ ID NO: 105 - protein, 29 aa, Severe acute respiratory syndrome coronavirus 2
DYYQLYSTQLSTDTGVEHVTFFIYNKIVD

SEQ ID NO: 106 - protein, 29 aa, Severe acute respiratory syndrome coronavirus 2
TFFIYNKIVDEPEEHVQIHTIDGSSGVVN

SEQ ID NO: 107 - protein, 28 aa, Severe acute respiratory syndrome coronavirus 2
TIDGSSGVVNPVMEPIYDEPTTTTSVPL

SEQ ID NO: 108 - protein, 50 aa, Severe acute respiratory syndrome coronavirus 2
FLYLYALVYFLQSINFVRIIMRLWLCWKCRSKNPLLYDANYFLCWHTNCY

SEQ ID NO: 109 - protein, 50 aa, Severe acute respiratory syndrome coronavirus 2
YFLCWHTNCYDYCIPYNSVTSSIVITSGDGTTSPISEHDYQIGGYTEKWE

SEQ ID NO: 110 - protein, 50 aa, Severe acute respiratory syndrome coronavirus 2
QIGGYTEKWESGVKDCVVLHSYFTSDYYQLYSTQLSTDTGVEHVTFFIYN

SEQ ID NO: 111 - protein, 51 aa, Severe acute respiratory syndrome coronavirus 2
VEHVTFFIYNKIVDEPEEHVQIHTIDGSSGVVNPVMEPIYDEPTTTTSVPL

SEQ ID NO: 112 - protein, 91 aa, Severe acute respiratory syndrome coronavirus 2
FLYLYALVYFLQSINFVRIIMRLWLCWKCRSKNPLLYDANYFLCWHTNCYDYCIPYNSVTSSIVITSGDGTTSPISEHDYQIGGYTEKWES

SEQ ID NO: 113 - protein, 91 aa, Severe acute respiratory syndrome coronavirus 2
QIGGYTEKWESGVKDCVVLHSYFTSDYYQLYSTQLSTDTGVEHVTFFIYNKIVDEPEEHVQIHTIDGSSGVVNPVMEPIYDEPTTTTSVPL

SEQ ID NO: 114 - protein, 171 aa, Severe acute respiratory syndrome coronavirus 2
FLYLYALVYFLQSINFVRIIMRLWLCWKCRSKNPLLYDANYFLCWHTNCYDYCIPYNSVTSSIVITSGDGTTSPISEHDYQIGGYTEKWESGVKDCVVLHSYFTSDYYQLYSTQLSTDTGVEHVTFFIYNKIVDEPEEHVQIHTIDGSSGVVNPVMEPIYDEPTTTTSVPL

SEQ ID NO: 115 - protein, 28 aa, Severe acute respiratory syndrome coronavirus 2
MKIILFLALITLATCELYHYQECVRGTT

SEQ ID NO: 116 - protein, 28 aa, Severe acute respiratory syndrome coronavirus 2
YQECVRGTTVLLKEPCSSGTYEGNSPFH

SEQ ID NO: 117 - protein, 28 aa, Severe acute respiratory syndrome coronavirus 2
GTYEGNSPFHPLADNKFALTCFSTQFAF

SEQ ID NO: 118 - protein, 29 aa, Severe acute respiratory syndrome coronavirus 2
LTCFSTQFAFACPDGVKHVYQLRARSVSP

SEQ ID NO: 119 - protein, 29 aa, Severe acute respiratory syndrome coronavirus 2
YQLRARSVSPKLFIRQEEVQELYSPIFLI

SEQ ID NO: 120 - protein, 28 aa, Severe acute respiratory syndrome coronavirus 2
QELYSPIFLIVAAIVFITLCFTLKRKTE

SEQ ID NO: 121 - protein, 48 aa, Severe acute respiratory syndrome coronavirus 2
MKIILFLALITLEATCELYHYQECVRGTTVLLKEPCSSGTYEGNSPFH

SEQ ID NO: 122 - protein, 47 aa, Severe acute respiratory syndrome coronavirus 2
GTYEGNSPFHPLADNKFALTCFSTQFAFACPDGVKHVYQLRARSVSP

SEQ ID NO: 123 - protein, 47 aa, Severe acute respiratory syndrome coronavirus 2
YQLRARSVSPKLFIRQEEVQELYSPIFLIVAAIVFITLCFTLKRKTE

SEQ ID NO: 124 - protein, 121 aa, Severe acute respiratory syndrome coronavirus 2
MKIILFLALITLATCELYHYQECVRGTTVLLKEPCSSGTYEGNSPFHPLADNKFALTCFSTQFAFACPDGVKHVYQLRARSVSPKLFIRQEEVQELYSPIFLIVAAIVFITLCFTLKRKTE

SEQ ID NO: 125 - protein, 30 aa, Severe acute respiratory syndrome coronavirus 2
SAVKRTIKGTHHWLLLTILTSLLVLVQSTQ

SEQ ID NO: 126 - protein, 30 aa, Severe acute respiratory syndrome coronavirus 2
SLLVLVQSTQWSLFFFLYENAFLPFAMGII

SEQ ID NO: 127 - protein, 30 aa, Severe acute respiratory syndrome coronavirus 2
AFLPFAMGIIAMSAFAMMFVKHKHAFLCLF

SEQ ID NO: 128 - protein, 30 aa, Severe acute respiratory syndrome coronavirus 2
KHKHAFLCLFLLPSLATVAYFNMVYMPASW

SEQ ID NO: 129 - protein, 30 aa, Severe acute respiratory syndrome coronavirus 2
FNMVYMPASWVMRIMTWLDMVDTSLSGFKL

SEQ ID NO: 130 - protein, 30 aa, Severe acute respiratory syndrome coronavirus 2
VDTSLSGFKLKDCVMYASAVVLLILMTART

SEQ ID NO: 131 - protein, 30 aa, Severe acute respiratory syndrome coronavirus 2
VLLILMTARTVYDDGARRVWTLMNVLTLVY

SEQ ID NO: 132 - protein, 30 aa, Severe acute respiratory syndrome coronavirus 2
TLMNVLTLVYKVYYGNALDQAISMWALIIS

SEQ ID NO: 133 - protein, 30 aa, Severe acute respiratory syndrome coronavirus 2
AISMWALIISVTSNYSGVVTTVMFLARGIV

SEQ ID NO: 134 - protein, 30 aa, Severe acute respiratory syndrome coronavirus 2
TVMFLARGIVFMCVEYCPIFFITGNTLQCI

SEQ ID NO: 135 - protein, 30 aa, Severe acute respiratory syndrome coronavirus 2
FITGNTLQCIMLVYCFLGYFCTCYFGLFCL

SEQ ID NO: 136 - protein, 30 aa, Severe acute respiratory syndrome coronavirus 2
CTCYFGLFCLLNRYFRLTLGVYDYLVSTQE

SEQ ID NO: 137 - protein, 30 aa, Severe acute respiratory syndrome coronavirus 2
VYDYLVSTQEFRYMNSQGLLPPKNSIDAFK

SEQ ID NO: 138 - protein, 30 aa, Severe acute respiratory syndrome coronavirus 2
PPKNSIDAFKLNIKLLGVGGKPCIKVATVQ

SEQ ID NO: 139 - protein, 28 aa, Severe acute respiratory syndrome coronavirus 2
SKMSDVKCTSVVLLSVLQQLRVESSSKL

SEQ ID NO: 140 - protein, 28 aa, Severe acute respiratory syndrome coronavirus 2
QLRVESSSKLWAQCVQLHNDILLAKDTT

SEQ ID NO: 141 - protein, 28 aa, Severe acute respiratory syndrome coronavirus 2
NDILLAKDTTEAFEKMVSLLSVLLSMQG

SEQ ID NO: 142 - protein, 29 aa, Severe acute respiratory syndrome coronavirus 2
LLSVLLSMQGAVDINKLCEEMLDNRATLQ

SEQ ID NO: 143 - protein, 50 aa, Severe acute respiratory syndrome coronavirus 2
SAVKRTIKGTHHWLLLTILTSLLVLVQSTQWSLFFFLYENAFLPFAMGII

SEQ ID NO: 144 - protein, 50 aa, Severe acute respiratory syndrome coronavirus 2
AFLPFAMGIIAMSAFAMMFVKHKHAFLCLFLLPSLATVAYFNMVYMPASW

SEQ ID NO: 145 - protein, 50 aa, Severe acute respiratory syndrome coronavirus 2
FNMVYMPASWVMRIMTWLDMVDTSLSGFKLKDCVMYASAVVLLILMTART

SEQ ID NO: 146 - protein, 50 aa, Severe acute respiratory syndrome coronavirus 2
VLLILMTARTVYDDGARRVWTLMNVLTLVYKVYYGNALDQAISMWALIIS

SEQ ID NO: 147 - protein, 50 aa, Severe acute respiratory syndrome coronavirus 2
AISMWALIISVTSNYSGVVTTVMFLARGIVFMCVEYCPIFFITGNTLQCI

SEQ ID NO: 148 - protein, 50 aa, Severe acute respiratory syndrome coronavirus 2
FITGNTLQCIMLVYCFLGYFCTCYFGLFCLLNRYFRLTLGVYDYLVSTQE

SEQ ID NO: 149 - protein, 50 aa, Severe acute respiratory syndrome coronavirus 2
VYDYLVSTQEFRYMNSQGLLPPKNSIDAFKLNIKLLGVGGKPCIKVATVQ

SEQ ID NO: 150 - protein, 46 aa, Severe acute respiratory syndrome coronavirus 2
SKMSDVKCTSVVLLSVLQQLRVESSSKLWAQCVQLHNDILLAKDTT

SEQ ID NO: 151 - protein, 47 aa, Severe acute respiratory syndrome coronavirus 2
NDILLAKDTTEAFEKMVSLLSVLLSMQGAVDINKLCEEMLDNRATLQ

SEQ ID NO: 152 - protein, 104 aa, Severe acute respiratory syndrome coronavirus 2
SAVKRTIKGTHHWLLLTILTSLLVLVQSTQWSLFFFLYENAFLPFAMGIIAMSAFAMMFVKHKHAFLCLFLLPSLATVAYFNMVYMPASWVMRIMTWLDMVDTS

SEQ ID NO: 153 - protein, 104 aa, Severe acute respiratory syndrome coronavirus 2
IMTWLDMVDTSLSGFKLKDCVMYASAVVLLILMTARTVYDDGARRVWTLMNVLTLVYKVYYGNALDQAISMWALIISVTSNYSGVVTTVMFLARGIVFMCVEYC

SEQ ID NO: 154 - protein, 104 aa, Severe acute respiratory syndrome coronavirus 2
RGIVFMCVEYCPIFFITGNTLQCIMLVYCFLGYFCTCYFGLFCLLNRYFRLTLGVYDYLVSTQEFRYMNSQGLLPPKNSIDAFKLNIKLLGVGGKPCIKVATVQ

SEQ ID NO: 155 - protein, 290 aa, Severe acute respiratory syndrome coronavirus 2
SAVKRTIKGTHHWLLLTILTSLLVLVQSTQWSLFFFLYENAFLPFAMGIIAMSAFAMMFVKHKHAFLCLFLLPSLATVAYFNMVYMPASWVMRIMTWLDMVDTSLSGFKLKDCVMYASAVVLLILMTARTVYDDGARRVWTLMNVLTLVYKVYYGNALDQAISMWALIISVTSNYSGVVTTVMFLARGIVFMCVEYCPIFFITGNTLQCIMLVYCFLGYFCTCYFGLFCLLNRYFRLTLGVYDYLVSTQEFRYMNSQGLLPPKNSIDAFKLNIKLLGVGGKPCIKVATVQ

SEQ ID NO: 156 - protein, 83 aa, Severe acute respiratory syndrome coronavirus 2
SKMSDVKCTSVVLLSVLQQLRVESSSKLWAQCVQLHNDILLAKDTTEAFEKMVSLLSVLLSMQGAVDINKLCEEMLDNRATLQ

SEQ ID NO: 157 - protein, 28 aa, Severe acute respiratory syndrome coronavirus 2
DLVYALRHFDEGNCDTLKEILVTYNCCD

SEQ ID NO: 158 - protein, 28 aa, Severe acute respiratory syndrome coronavirus 2
EILVTYNCCDDDYFNKKDWYDFVENPDI

SEQ ID NO: 159 - protein, 28 aa, Severe acute respiratory syndrome coronavirus 2
WYDFVENPDILRVYANLGERVRQALLKT

SEQ ID NO: 160 - protein, 28 aa, Severe acute respiratory syndrome coronavirus 2
ERVRQALLKTVQFCDAMRNAGIVGVLTL

SEQ ID NO: 161 - protein, 28 aa, Severe acute respiratory syndrome coronavirus 2
NAGIVGVLTLDNQDLNGNWYDFGDFIQT

SEQ ID NO: 162 - protein, 28 aa, Severe acute respiratory syndrome coronavirus 2
WYDFGDFIQTTPGSGVPVVDSYYSLLMP

SEQ ID NO: 163 - protein, 28 aa, Severe acute respiratory syndrome coronavirus 2
VDSYYSLLMPILTLTRALTAESHVDTDL

SEQ ID NO: 164 - protein, 29 aa, Severe acute respiratory syndrome coronavirus 2
TAESHVDTDLTKPYIKWDLLKYDFTEERL

SEQ ID NO: 165 - protein, 29 aa, Severe acute respiratory syndrome coronavirus 2
LKYDFTEERLKLFDRYFKYWDQTYHPNCV

SEQ ID NO: 166 - protein, 28 aa, Severe acute respiratory syndrome coronavirus 2
WDQTYHPNCVNCLDDRCILHCANFNVLF

SEQ ID NO: 167 - protein, 29 aa, Severe acute respiratory syndrome coronavirus 2
LHCANFNVLFSTVFPPTSFGPLVRKIFVD

SEQ ID NO: 168 - protein, 29 aa, Severe acute respiratory syndrome coronavirus 2
GPLVRKIFVDGVPFVVSTGYHFRELGVVH

SEQ ID NO: 169 - protein, 30 aa, Severe acute respiratory syndrome coronavirus 2
YHFRELGVVHNQDVNLHSSRLSFKELLVYA

SEQ ID NO: 170 - protein, 50 aa, Severe acute respiratory syndrome coronavirus 2
DLVYALRHFDEGNCDTLKEILVTYNCCDDDYFNKKDWYDFVENPDILRVY

SEQ ID NO: 171 - protein, 50 aa, Severe acute respiratory syndrome coronavirus 2
VENPDILRVYANLGERVRQALLKTVQFCDAMRNAGIVGVLTLDNQDLNGN

SEQ ID NO: 172 - protein, 50 aa, Severe acute respiratory syndrome coronavirus 2
TLDNQDLNGNWYDFGDFIQTTPGSGVPVVDSYYSLLMPILTLTRALTAES

SEQ ID NO: 173 - protein, 50 aa, Severe acute respiratory syndrome coronavirus 2
TLTRALTAESHVDTDLTKPYIKWDLLKYDFTEERLKLFDRYFKYWDQTYH

SEQ ID NO: 174 - protein, 50 aa, Severe acute respiratory syndrome coronavirus 2
YFKYWDQTYHPNCVNCLDDRCILHCANFNVLFSTVFPPTSFGPLVRKIFV

SEQ ID NO: 175 - protein, 50 aa, Severe acute respiratory syndrome coronavirus 2
FGPLVRKIFVDGVPFVVSTGYHFRELGVVHNQDVNLHSSRLSFKELLVYA

SEQ ID NO: 176 - protein, 130 aa, Severe acute respiratory syndrome coronavirus 2
DLVYALRHFDEGNCDTLKEILVTYNCCDDDYFNKKDWYDFVENPDILRVYANLGERVRQALLKTVQFCDAMRNAGIVGVLTLDNQDLNGNWYDFGDFIQTTPGSGVPVVDSYYSLLMPILTLTRALTAES

SEQ ID NO: 177 - protein, 130 aa, Severe acute respiratory syndrome coronavirus 2
TLTRALTAESHVDTDLTKPYIKWDLLKYDFTEERLKLFDRYFKYWDQTYHPNCVNCLDDRCILHCANFNVLFSTVFPPTSFGPLVRKIFVDGVPFVVSTGYHFRELGVVHNQDVNLHSSRLSFKELLVYA

SEQ ID NO: 178 - protein, 250 aa, Severe acute respiratory syndrome coronavirus 2
DLVYALRHFDEGNCDTLKEILVTYNCCDDDYFNKKDWYDFVENPDILRVYANLGERVRQALLKTVQFCDAMRNAGIVGVLTLDNQDLNGNWYDFGDFIQTTPGSGVPVVDSYYSLLMPILTLTRALTAESHVDTDLTKPYIKWDLLKYDFTEERLKLFDRYFKYWDQTYHPNCVNCLDDRCILHCANFNVLFSTVFPPTSFGPLVRKIFVDGVPFVVSTGYHFRELGVVHNQDVNLHSSRLSFKELLVYA

SEQ ID NO: 179 - protein, 29 aa, Severe acute respiratory syndrome coronavirus 2
SYEDQDALFAYTKRNVIPTITQMNLKYAI

SEQ ID NO: 180 - protein, 29 aa, Severe acute respiratory syndrome coronavirus 2
TITQMNLKYAISAKNRARTVAGVSICSTM

SEQ ID NO: 181 - protein, 29 aa, Severe acute respiratory syndrome coronavirus 2
VAGVSICSTMNRQFHQKLLKSIAATRGAT

SEQ ID NO: 182 - protein, 29 aa, Severe acute respiratory syndrome coronavirus 2
LKSIAATRGATVVIGTSKFYGGWHNMLKT

SEQ ID NO: 183 - protein, 30 aa, Severe acute respiratory syndrome coronavirus 2
YGGWHNMLKTVYSDVENPHLMGWDYPKCDR

SEQ ID NO: 184 - protein, 29 aa, Severe acute respiratory syndrome coronavirus 2
MGWDYPKCDRAMPNMLRIMASLVLARKHT

SEQ ID NO: 185 - protein, 29 aa, Severe acute respiratory syndrome coronavirus 2
ASLVLARKHTTCCSLSHRFYRLANECAQV

SEQ ID NO: 186 - protein, 31 aa, Severe acute respiratory syndrome coronavirus 2
YRLANECAQVLSEMVMCGGSLYVKPGGTSSG

SEQ ID NO: 187 - protein, 31 aa, Severe acute respiratory syndrome coronavirus 2
YVKPGGTSSGDATTAYANSVFNICQAVTANV

SEQ ID NO: 188 - protein, 30 aa, Severe acute respiratory syndrome coronavirus 2
NICQAVTANVNALLSTDGNKIADKYVRNLQ

SEQ ID NO: 189 - protein, 30 aa, Severe acute respiratory syndrome coronavirus 2
IADKYVRNLQHRLYECLYRNRDVDTDFVNE

SEQ ID NO: 190 - protein, 30 aa, Severe acute respiratory syndrome coronavirus 2
RDVDTDFVNEFYAYLRKHFSMMILSDDAVV

SEQ ID NO: 191 - protein, 30 aa, Severe acute respiratory syndrome coronavirus 2
MMILSDDAVVCFNSTYASQGLVASIKNFKS

SEQ ID NO: 192 - protein, 30 aa, Severe acute respiratory syndrome coronavirus 2
LVASIKNFKSVLYYQNNVFMSEAKCWTETD

SEQ ID NO: 193 - protein, 30 aa, Severe acute respiratory syndrome coronavirus 2
SEAKCWTETDLTKGPHEFCSQHTMLVKQGD

SEQ ID NO: 194 - protein, 30 aa, Severe acute respiratory syndrome coronavirus 2
QHTMLVKQGDDYVYLPYPDPSRILGAGCFV

SEQ ID NO: 195 - protein, 29 aa, Severe acute respiratory syndrome coronavirus 2
SRILGAGCFVDDIVKTDGTLMIERFVSLA

SEQ ID NO: 196 - protein, 29 aa, Severe acute respiratory syndrome coronavirus 2
LMIERFVSLAIDAYPLTKHPNQEYADVFH

SEQ ID NO: 197 - protein, 29 aa, Severe acute respiratory syndrome coronavirus 2
PNQEYADVFHLYLQYIRKLHDELTGHMLD

SEQ ID NO: 198 - protein, 29 aa, Severe acute respiratory syndrome coronavirus 2
HDELTGHMLDMYSVMLTNDNTSRYWEPEF

SEQ ID NO: 199 - protein, 49 aa, Severe acute respiratory syndrome coronavirus 2
SYEDQDALFAYTKRNVIPTITQMNLKYAISAKNRARTVAGVSICSTMTN

SEQ ID NO: 200 - protein, 49 aa, Severe acute respiratory syndrome coronavirus 2
GVSICSTMTNRQFHQKLLKSIAATRGATVVIGTSKFYGGWHNMLKTVYS

SEQ ID NO: 201 - protein, 49 aa, Severe acute respiratory syndrome coronavirus 2
WHNMLKTVYSDVENPHLMGWDYPKCDRAMPNMLRIMASLVLARKHTTCC

SEQ ID NO: 202 - protein, 49 aa, Severe acute respiratory syndrome coronavirus 2
VLARKHTTCCSLSHRFYRLANECAQVLSEMVMCGGSLYVKPGGTSSGDA

SEQ ID NO: 203 - protein, 49 aa, Severe acute respiratory syndrome coronavirus 2
KPGGTSSGDATTAYANSVFNICQAVTANVNALLSTDGNKIADKYVRNLQ

SEQ ID NO: 204 - protein, 49 aa, Severe acute respiratory syndrome coronavirus 2
IADKYVRNLQHRLYECLYRNRDVDTDFVNEFYAYLRKHFSMMILSDDAV

SEQ ID NO: 205 - protein, 49 aa, Severe acute respiratory syndrome coronavirus 2
SMMILSDDAVVCFNSTYASQGLVASIKNFKSVLYYQNNVFMSEAKCWTE

SEQ ID NO: 206 - protein, 49 aa, Severe acute respiratory syndrome coronavirus 2
FMSEAKCWTETDLTKGPHEFCSQHTMLVKQGDDYVYLPYPDPSRILGAG

SEQ ID NO: 207 - protein, 49 aa, Severe acute respiratory syndrome coronavirus 2
PDPSRILGAGCFVDDIVKTDGTLMIERFVSLAIDAYPLTKHPNQEYADV

SEQ ID NO: 208 - protein, 50 aa, Severe acute respiratory syndrome coronavirus 2
KHPNQEYADVFHLYLQYIRKLHDELTGHMLDMYSVMLTNDNTSRYWEPEF

SEQ ID NO: 209 - protein, 110 aa, Severe acute respiratory syndrome coronavirus 2
SYEDQDALFAYTKRNVIPTITQMNLKYAISAKNRARTVAGVSICSTMTNRQFHQKLLKSIAATRGATVVIGTSKFYGGWHNMLKTVYSDVENPHLMGWDYPKCDRAMPNM

SEQ ID NO: 210 - protein, 109 aa, Severe acute respiratory syndrome coronavirus 2
YPKCDRAMPNMLRIMASLVLARKHTTCCSLSHRFYRLANECAQVLSEMVMCGGSLYVKPGGTSSGDATTAYANSVFNICQAVTANVNALLSTDGNKIADKYVRNLQHRL

SEQ ID NO: 211 - protein, 110 aa, Severe acute respiratory syndrome coronavirus 2
DKYVRNLQHRLYECLYRNRDVDTDFVNEFYAYLRKHFSMMILSDDAVVCFNSTYASQGLVASIKNFKSVLYYQNNVFMSEAKCWTETDLTKGPHEFCSQHTMLVKQGDDY

SEQ ID NO: 212 - protein, 105 aa, Severe acute respiratory syndrome coronavirus 2
HTMLVKQGDDYVYLPYPDPSRILGAGCFVDDIVKTDGTLMIERFVSLAIDAYPLTKHPNQEYADVFHLYLQYIRKLHDELTGHMLDMYSVMLTNDNTSRYWEPEF

SEQ ID NO: 213 - protein, 401 aa, Severe acute respiratory syndrome coronavirus 2
SYEDQDALFAYTKRNVIPTITQMNLKYAISAKNRARTVAGVSICSTMTNRQFHQKLLKSIAATRGATVVIGTSKFYGGWHNMLKTVYSDVENPHLMGWDYPKCDRAMPNMLRIMASLVLARKHTTCCSLSHRFYRLANECAQVLSEMVMCGGSLYVKPGGTSSGDATTAYANSVFNICQAVTANVNALLSTDGNKIADKYVRNLQHRLYECLYRNRDVDTDFVNEFYAYLRKHFSMMILSDDAVVCFNSTYASQGLVASIKNFKSVLYYQNNVFMSEAKCWTETDLTKGPHEFCSQHTMLVKQGDDYVYLPYPDPSRILGAGCFVDDIVKTDGTLMIERFVSLAIDAYPLTKHPNQEYADVFHLYLQYIRKLHDELTGHMLDMYSVMLTNDNTSRYWEPEF

SEQ ID NO: 214 - protein, 29 aa, Betacoronavirus 1
TADEAIKFLKEWNFSLGIILLFITIILQF

SEQ ID NO: 215 - protein, 29 aa, Betacoronavirus 1
LLFITIIILQFGYTSRSMFVYVIKMIILW

SEQ ID NO: 216 - protein, 29 aa, Betacoronavirus 1
VYVIKMIILWLMWPLTIILTIFNCVYALN

SEQ ID NO: 217 - protein, 29 aa, Betacoronavirus 1
TIFNCVYALNNVYLGLSIVFTIVAIIMWI

SEQ ID NO: 218 - protein, 29 aa, Betacoronavirus 1
FTIVAIIMWIVYFVNSIRLFIRTGSFWSF

SEQ ID NO: 219 - protein, 29 aa, Betacoronavirus 1
FIRTGSFWSFNPETNNLMCIDMKGTMYVR

SEQ ID NO: 220 - protein, 28 aa, Betacoronavirus 1
IDMKGTMYVRPIIEDYHTLTVTIIRGHL

SEQ ID NO: 221 - protein, 28 aa, Betacoronavirus 1
LTVTIIRGHLYIQGIKLGTGYSLADLPA

SEQ ID NO: 222 - protein, 28 aa, Betacoronavirus 1
TGYSLADLPAYMTVAKVTHLCTYKRGFL

SEQ ID NO: 223 - protein, 28 aa, Betacoronavirus 1
HLCTYKRGFLDRISDTSGFAVYVKSKVG

SEQ ID NO: 224 - protein, 28 aa, Betacoronavirus 1
FAVYVKSKVGNYRLPSTQKGSGMDTALL

SEQ ID NO: 225 - protein, 23 aa, Human coronavirus HKU11
NNAFLAFSIVFTIISIVIWILYF

SEQ ID NO: 226 - protein, 27 aa, Human coronavirus HKU11
KVQVLCTYKRAFLDKLDVNSGFAVFVK

SEQ ID NO: 227 - protein, 44 aa, Betacoronavirus 1
TADEAIKFLKEWNFSLGIILLFITIILQFGYTSRSMFVYVIKMI

SEQ ID NO: 228 - protein, 44 aa, Betacoronavirus 1
SMFVYVIKMIILWLMWPLTIILTIFNCVYALNNVYLGLSIVFTI

SEQ ID NO: 229 - protein, 44 aa, Betacoronavirus 1
YLGLSIVFTIVAIIMWIVYFVNSIRLFIRTGSFWSFNPETNNLM

SEQ ID NO: 230 - protein, 44 aa, Betacoronavirus 1
SFNPETNNLMCIDMKGTMFVRPIIEDYHTLTVTIIRGHLYIQGI

SEQ ID NO: 231 - protein, 45 aa, Betacoronavirus 1
IRGHLYIQGIKLGTGYSLADLPAYMTVAKVTYLCTYKRGFLDKIS

SEQ ID NO: 232 - protein, 42 aa, Betacoronavirus 1
YKRGFLDKISDTSGFAVYVKSKVGNYRLPSTQKGSGMDTALL

SEQ ID NO: 233 - protein, 50 aa, Artificial Sequence
  Description of Artificial Sequence Synthetic polypeptide
NNAFLAFSIVFTIISIVIWILYFKVQVLCTYKRAFLDKLDVNSGFAVFVK

SEQ ID NO: 234 - protein, 138 aa, Betacoronavirus 1
TADEAIKFLKEWNFSLGIILLFITIILQFGYTSRSMFVYVIKMIILWLMWPLTIILTIFNCVYALNNVYLGLSIVFTIVAIIMWIVYFVNSIRLFIRTGSFWSFNPETNNLMCIDMKGTMYVRPIIEDYHTLTVTIIR

SEQ ID NO: 235 - protein, 136 aa, Artificial Sequence
  Description of Artificial Sequence Synthetic polypeptide
DYHTLTVTIIRGHLYIQGIKLGTGYSLADLPAYMTVAKVTHLCTYKRGFLDRISDTSGFAVYVKSKVGNYRLPSTQKGSGMDTALLNNAFLAFSIVFTIISIVIWILYFKVQVLCTYKRAFLDKLDVNSGFAVFVK

SEQ ID NO: 236 - protein, 263 aa, Artificial Sequence
  Description of Artificial Sequence Synthetic polypeptide
TADEAIKFLKEWNFSLGIILLFITIILQFGYTSRSMFVYVIKMIILWLMWPLTIILTIFNCVYALNNVYLGLSIVFTIVAIIMWIVYFVNSIRLFIRTGSFWSFNPETNNLMCIDMKGTMYVRPIIEDYHTLTVTIIRGHLYIQGIKLGTGYSLADLPAYMTVAKVTHLCTYKRGFLDRISDTSGFAVYVKSKVGNYRLPSTQKGSGMDTALLNNAFLAFSIVFTIISIVIWILYFKVQVLCTYKRAFLDKLDVNSGFAVFVK

SEQ ID NO: 237 - protein, 25 aa, Betacoronavirus 1
SWFSGITQFQKGKEFEFVEGQGVPI

SEQ ID NO: 238 - protein, 33 aa, Betacoronavirus 1
GYWYRHNRRSFKTADGNQRQLLPRWYFYYLGTG

SEQ ID NO: 239 - protein, 33 aa, Betacoronavirus 1
RWYFYYLGTGPHAKDQYGDIDGVYWVASNQAVD

SEQ ID NO: 240 - protein, 32 aa, Betacoronavirus 1
YWVASNQADVNTPADIVDRDPSSDEAIPTRFP

SEQ ID NO: 241 - protein, 33 aa, Betacoronavirus 1
SDEAIPTRFPPGTVLPQGYYIEGSGRSAPNSRS

SEQ ID NO: 242 - protein, 33 aa, Betacoronavirus 1
SGRSAPNSRSTSRTSSRASSAGSRSRANSGNRT

SEQ ID NO: 243 - protein, 32 aa, Betacoronavirus 1
RSRANSGNRTPTSGVTPDMADQIASLVLAKLG

SEQ ID NO: 244 - protein, 30 aa, Betacoronavirus 1
IASLVLAKLGKDATKPQQVTKHTAKEVRQK

SEQ ID NO: 245 - protein, 33 aa, Betacoronavirus 1
PRQKRSPNKQCTVQQCFGKRGPNQNFGGGEMLK

SEQ ID NO: 246 - protein, 33 aa, Betacoronavirus 1
QNFGGGEMLKLGTSDPQFPILAELAPTAGAFFF

SEQ ID NO: 247 - protein, 34 aa, Artificial Sequence
  Description of Artificial Sequence Synthetic polypeptide
LAPTAGAFFFGSRLELAKVQNLSGNELRYNGAIR

SEQ ID NO: 248 - protein, 32 aa, Betacoronavirus 1
ELRYNGAIRFDSTLSGFETIMKVLNENLNAYQ

SEQ ID NO: 249 - protein, 26 aa, Human coronavirus HKU11
PYANASYGESLEGVFWVANHQADTST

SEQ ID NO: 250 - protein, 24 aa, Human coronavirus HKU11
VANHQADTSTPSDVSSRDPTTQEA

SEQ ID NO: 251 - protein, 32 aa, Human coronavirus HKU11
RPGSRSQSRGPNNRSLSRSNSNFRHSDSIVKP

SEQ ID NO: 252 - protein, 17 aa, Human coronavirus HKU11
SKLDLVKRDSEADSPVK

SEQ ID NO: 253 - protein, 46 aa, Artificial Sequence
  Description of Artificial Sequence Synthetic polypeptide
SWFSGITQFQKGKEFEFVEGQGVPIGYWYRHNRRSFKTADGNQRQL

SEQ ID NO: 254 - protein, 46 aa, Betacoronavirus 1
KTADGNQRQLLPRWYFYYLGTGPHAKDQYGTDIDGVYWVASNQADV

SEQ ID NO: 255 - protein, 46 aa, Betacoronavirus 1
YWVASNQADVNTPADIVDRDPSSDEAIPTRFPPGTVLPQGYYIEGS

SEQ ID NO: 256 - protein, 46 aa, Betacoronavirus 1
LPQGYYIEGSGRSAPNSRSTSRTSRTSSRASSAGSRSRANSGNRTP

SEQ ID NO: 257 - protein, 46 aa, Betacoronavirus 1
SRANSGNRTPTSGVTPDMADQIASLVLAKLGKDATKPQQVTKHTAK

SEQ ID NO: 258 - protein, 46 aa, Artificial Sequence
  Description of Artificial Sequence Synthetic polypeptide
PQQVTKHTAKEVRQKPRQKRSPNKQCTVQQCFGKRGPNQNFGGGEM

SEQ ID NO: 259 - protein, 46 aa, Betacoronavirus 1
PNQNFGGGEMLKLGTSDPQFPILAELAPTAGAFFFGSRLELAKVQN

SEQ ID NO: 260 - protein, 46 aa, Artificial Sequence
  Description of Artificial Sequence Synthetic polypeptide
SRLELAKVQNLSGNELRYNGAIRFDSTLSGFETIMKVLNENLNAYQ

SEQ ID NO: 261 - protein, 40 aa, Human coronavirus HKU11
PYANASYGESLEGVFWVANHQADTSTPSDVSSRDPTTQEA

SEQ ID NO: 262 - protein, 49 aa, Artificial Sequence
  Description of Artificial Sequence Synthetic polypeptide
RPGSRSQSRGPNNRSLSRSNSNFRHSDSIVKPSKLDLVKRDSEADSPVK

SEQ ID NO: 263 - protein, 111 aa, Artificial Sequence
  Description of Artificial Sequence Synthetic polypeptide
SWFSGITQFQKGKEFEFVEGQGVPIGYWYRHNRRSFKTADGNQRQLLPRWYFYYLGTGPHAKDQYGTDIDGVYWVASNQADVNTPADIVDRDPSSDEAIPTRFPPGTVLPQ

SEQ ID NO: 264 - protein, 106 aa, Artificial Sequence
  Description of Artificial Sequence Synthetic polypeptide
TRFPPGTVLPQGYYIEGSGRSAPNSRSTSRTSSRASSAGSRSRANSGNRTPTSGVTPDMADQIASLVLAKLGKDATKPQQVTKHTAKEVRQKPRQKRSPNKQCTVQ

SEQ ID NO: 265 - protein, 99 aa, Betacoronavirus 1
KRSPNKQCTVQQCFGKRGPNQNFGGGEMLKGTSDPQFPILAELAPTAGAFFFGSRLELAKVQNLSGNELRYNGAIRFDSTLSGFETIMKVLNENLNAYQ

SEQ ID NO: 266 - protein, 89 aa, Artificial Sequence
  Description of Artificial Sequence Synthetic polypeptide
PYANASYGESLEGVFWVANHQADTSTPSDVSSRDPTTQEARPGSRSQSRGPNNRSLSRSNSNFRHSDSIVKPSKLDLVKRDSEADSPVK

SEQ ID NO: 267 - protein, 383 aa, Artificial Sequence
  Description of Artificial Sequence Synthetic polypeptide
SWFSGITQFQKGKEFEFVEGQGVPIGYWYRHNRRSFKTADGNQRQLLPRWYFYYLGTGPHAKDQYGDIDGVYWVASNQADVNTPADIVDRDPSSDEAIPTRFPPGTVLPQGYYIEGSGRSAPNSRSTSRTSSRASSAGSRSRANSGNRTPTSGVTPDMADQIASLVLAKLGKDATKPQQVTKHTAKEVRQKPRQKRSPNKQCTVQQCFGKRGPNQNFGGGEMLKLGTSDPQFPILAELAPTAGAFFFGSRLELAKVQNLSGNELRYNGAIRFDSTLSGFETIMKVLNENLNAYQPYANASYGESLEGVFWVANHQADTSTPSDVSSRDPTTQEARPGSRSQSRGPNNRSLSRSNSNFRHSDSIVKPSKLDLVKRDSEADSPVK

SEQ ID NO: 268 - protein, 32 aa, Betacoronavirus 1
SKASSRSAIEDLLFDKVKLSDVGFVEAYNNCT

SEQ ID NO: 269 - protein, 32 aa, Betacoronavirus 1
GFVEAYNNCTGGAEIRDLICVQSYKGIKVLPP

SEQ ID NO: 270 - protein, 32 aa, Betacoronavirus 1
SYKGIKVLPPLLSENQISGYTLAATSASLFPP

SEQ ID NO: 271 - protein, 32 aa, Betacoronavirus 1
AATSASLFPPWTAAAGVPFYLNVQYRINGLGV

SEQ ID NO: 272 - protein, 32 aa, Betacoronavirus 1
VQYRINGLGVTMDVLSQNQKLIANAFNNALYA

SEQ ID NO: 273 - protein, 32 aa, Betacoronavirus 1
ANAFNNALYAIQEGFDATNSALVKIQAVVNAN

SEQ ID NO: 274 - protein, 32 aa, Betacoronavirus 1
VKIQAVVNANAEALNNLLQQLSNRFGAISASL

SEQ ID NO: 275 - protein, 33 aa, Betacoronavirus 1
NRFGAISASLQEILSRLDALEAEAQIDRLINGR

SEQ ID NO: 276 - protein, 33 aa, Betacoronavirus 1
AQIDRLINGRLTALNAYVSQQLSDSTLVKFSAA

SEQ ID NO: 277 - protein, 33 aa, Betacoronavirus 1
DSTLVKFSAAQAMEKVNECVKSQSSRINFCGNG

SEQ ID NO: 278 - protein, 34 aa, Betacoronavirus 1
QSSRINFCGNGNHIISLVQNAPYGLYFIHFSYVP

SEQ ID NO: 279 - protein, 32 aa, Betacoronavirus 1
PNLPDFKEELDQWFKNQTSVAPDLSLDYINVT

SEQ ID NO: 280 - protein, 32 aa, Betacoronavirus 1
DLSLDYINVTFLDLQVEMNRLQEAIKVLNQSY

SEQ ID NO: 281 - protein, 31 aa, Betacoronavirus 1
EAIKVLNQSYINLKDIGTYEYYVKWPWYVWL

SEQ ID NO: 282 - protein, 30 aa, Human coronavirus HKU11
PKLSDFESELSHWFKNQTSIAPNLTLNLHT

SEQ ID NO: 283 - protein, 32 aa, Human coronavirus HKU11
APNLTLNLHTINATFLDLYYEMNLIQESIKSL

SEQ ID NO: 284 - protein, 45 aa, Betacoronavirus 1
SKASSRSAIEDLLFDKVKLSDVGFVEAYNNCTGGAEIRDLICVQS

SEQ ID NO: 285 - protein, 46 aa, Betacoronavirus 1
EIRDLICVQSYKGIKVLPPLLSENQISGYTLAATSASLFPPWTAAA

SEQ ID NO: 286 - protein, 45 aa, Betacoronavirus 1
SLFPPWTAAAGVPFYLNVQYRINGLGVTMDVLSQNQKLIANAFNN

SEQ ID NO: 287 - protein, 45 aa, Betacoronavirus 1
QKLIANAFNNALYAIQEGFDATNSALVKIQAVVNANAEALNNLLQ

SEQ ID NO: 288 - protein, 45 aa, Betacoronavirus 1
NAEALNNLLQQLSNRFGAISASLQEILSRLDALEAEAQIDRLING

SEQ ID NO: 289 - protein, 45 aa, Betacoronavirus 1
EAQIDRLINGRLTALNAYVSQQLSDSTLVKFSAAQAMEKVNECKS

SEQ ID NO: 290 - protein, 45 aa, Betacoronavirus 1
AMEKVNECVKSQSSRINFCGNGNHIISLVQNAPYGLYFIHFSYVP

SEQ ID NO: 291 - protein, 42 aa, Betacoronavirus 1
PNLPDFKEELDQWFKNQTSVAPDLSLDYINVTFLDLQVEMNR

SEQ ID NO: 292 - protein, 43 aa, Betacoronavirus 1
FLDLQVEMNRLQEAIKVLNQSYINLKDIGTYEYYVKWPWYVWL

SEQ ID NO: 293 - protein, 47 aa, Human coronavirus HKU11
PKLSDFESELSHWFKNQTSIAPNLTLNLHTINATFLMNLIQESIKSL

SEQ ID NO: 294 - protein, 136 aa, Betacoronavirus 1
SKASSRSAIEDLLFDKVKLSDVGFVEAYNNCTGGAEIRDLICVQSYKGIKVLPPLLSENQISGYTLAATSASLFPPWTAAAGVPFYLNVQYRINGLGVTMDVLSQNQKLIANAFNNALYAIQEGFDATNSALVKIQ

SEQ ID NO: 295 - protein, 132 aa, Betacoronavirus 1
FDATNSALVKIQAVVNANAEALNNLLQQLSNRFGAISASLQEILSRLDALEAEAQIDRLINGRLTALNAYVSQQLSDSTLVKFSAAQAMEKVNECVKSQSSRINFCGNGNHIISLVQNAPYGLYFIHFSYVP

SEQ ID NO: 296 - protein, 120 aa, Artificial Sequence
  Description of Artificial Sequence Synthetic polypeptide
PNLPDFKEELDQWFKNQTSVAPDLSDYINVFLDLQVEMNRLQEAIKVLNQSYINLKDIGTYEYYVKWPWYVWLPKLSDFESELSHWFKNQTSIAPNLTLNLHTINATFLMNLIQESIKSL

SEQ ID NO: 297 - protein, 32 aa, Betacoronavirus 1
FMRFYIIIASFIKLFSLFRHVAYGCSKSGCLF

SEQ ID NO: 298 - protein, 32 aa, Betacoronavirus 1
YGCSKSGCLFYKRNRSLRVKCSTIVGGMIRYY

SEQ ID NO: 299 - protein, 30 aa, Betacoronavirus 1
TIVGGMIRYYDVMANGGTGFCSKHQWNCID

SEQ ID NO: 300 - protein, 31 aa, Betacoronavirus 1
CSKHQWNCIDCDSYKPGNTFITVEAALDLSK

SEQ ID NO: 301 - protein, 32 aa, Artificial Sequence
  Description of Artificial Sequence Synthetic polypeptide
TVEAALDLSKELKRPIQPTDNAAVFYAQSLFR

SEQ ID NO: 302 - protein, 31 aa, Betacoronavirus 1
AVFYAQSLFRPILMVDKNLITTANTGTSVTE

SEQ ID NO: 303 - protein, 31 aa, Betacoronavirus 1
TANTGTSVTETMFDVYVDTFLSMFDVDKKSL

SEQ ID NO: 304 - protein, 32 aa, Artificial Sequence
  Description of Artificial Sequence Synthetic polypeptide
SMFDVDKKSLNALIATAELTDESCNNLVPTYL

SEQ ID NO: 305 - protein, 31 aa, Betacoronavirus 1
SCNNLVPTYLKSDNIVAADLGVLIQNSAKHV

SEQ ID NO: 306 - protein, 29 aa, Betacoronavirus 1
VLIQNSAKHVQGNVAKIAGVSCIWSVDAF

SEQ ID NO: 307 - protein, 42 aa, Betacoronavirus 1
FMRFYIIIASFIKLFSLFRHVAYGCSKSGCLFCYKRNRSLRV

SEQ ID NO: 308 - protein, 42 aa, Betacoronavirus 1
CYRNRSLRVKCSTIVGGMIRYYDVMANGGTGFCSKHQWNCID

SEQ ID NO: 309 - protein, 41 aa, Betacoronavirus 1
CSKHQWNCIDCDSYKPGNTFITVEAALDLSKELKRPIQPTD

SEQ ID NO: 310 - protein, 44 aa, Betacoronavirus 1
NAAVFYAQSLFRPILMVDKNLITTANTGTSVTETMFDVYVDTFL

SEQ ID NO: 311 - protein, 50 aa, Artificial Sequence
  Description of Artificial Sequence Synthetic polypeptide
MFDVYVDTFLSMFDVDKKSLNALIATAELTDESCNNLVPTYLKSDNIVAA

SEQ ID NO: 312 - protein, 42 aa, Betacoronavirus 1
YLKSDNIVAADLGVLIQNSAKHVQGNVAKIAGVSCIWSVDAF

SEQ ID NO: 313 - protein, 106 aa, Betacoronavirus 1
FMRFYIIIASFIKLFSLFRHVAYGCSKSGCLFCYKRNRSLRVKCSTIVGGMIRYYDVMANGGTGFCSKHQWNCIDCDSYKPGNTFITVEAALDLSKELKRPIQPTD

SEQ ID NO: 314 - protein, 116 aa, Artificial Sequence
  Description of Artificial Sequence Synthetic polypeptide
NAAVFYAQSLFRPILMVDKNLITTANTGTSVTETMFDVYVDTFLSMFDVDKKSLNALIATAELTDESCNNLVPTYLKSDNIVAADLGVLIQNSAKHVQGNVAKIAGVSCIWSVDAF

SEQ ID NO: 315 - protein, 222 aa, Artificial Sequence
  Description of Artificial Sequence Synthetic polypeptide
FMRFYIIIASFIKLFSLFRHVAYGCSKSGCLFCYKRNRSLRVKCSTIVGGMIRYYDVMANGGTGFCSKHQWNCIDCDSYKPGNTFITVEAALDLSKELKRPIQPTDNAAVFYAQSLFRPILMVDKNLITTANTGTSVTETMFDVYVDTFLSMFDVDKKSLNALIATAELTDESCNNLVPTYLKSDNIVAADLGVLIQNSAKHVQGNVAKIAGVSCIWSVDAF

SEQ ID NO: 316 - protein, 28 aa, Betacoronavirus 1
SADGVQCYTPHSQISYSNFYASGCVLSS

SEQ ID NO: 317 - protein, 26 aa, Betacoronavirus 1
FYASGCVLSSACTMFMADGSPQPYCY

SEQ ID NO: 318 - protein, 26 aa, Betacoronavirus 1
SLVPHVRYNLANAKGFIRFPEVLREG

SEQ ID NO: 319 - protein, 26 aa, Betacoronavirus 1
IRFPEVLREGLVRIVRTRSMSYCRVG

SEQ ID NO: 320 - protein, 25 aa, Betacoronavirus 1
TRSMSYCRVGLCEEADEGICFNFNG

SEQ ID NO: 321 - protein, 25 aa, Betacoronavirus 1
DEGICFNFNGSWVLNNDYYRSLPGT

SEQ ID NO: 322 - protein, 25 aa, Betacoronavirus 1
NDYYRSLPGTFCGRDVFDLIYQLFK

SEQ ID NO: 323 - protein, 25 aa, Betacoronavirus 1
VFDLIYQLFKGLAQPVDFLALTASS

SEQ ID NO: 324 - protein, 25 aa, Betacoronavirus 1
VDFLALTASSIAGAILAVIVVLVFY

SEQ ID NO: 325 - protein, 26 aa, Betacoronavirus 1
LAVIVVLVFYYLIKLKRAFGDYTSVV

SEQ ID NO: 326 - protein, 30 aa, Betacoronavirus 1
NRYLSLYNKYRYYSGKMDTAAYREAACSQL

SEQ ID NO: 327 - protein, 30 aa, Betacoronavirus 1
AYREAACSQLAKAMDTFTNNNGSDVLYQPP

SEQ ID NO: 328 - protein, 45 aa, Betacoronavirus 1
SADGVQCYTPHSQISYSNFYASGCVLSSACTMFTMADGSPQPYCY

SEQ ID NO: 329 - protein, 46 aa, Betacoronavirus 1
SLVPHVRYNLANAKGFIRFPEVLREGLVRIVRTRSMSYCRVGLCEE

SEQ ID NO: 330 - protein, 44 aa, Betacoronavirus 1
SYCRVGLCEEADEGICFNFNGSWVLNNDYYRSLPGTFCGRDVFD

SEQ ID NO: 331 - protein, 45 aa, Betacoronavirus 1
GTFCGRDVFDLIYQLFKGLAQPVDFLALTASSIAGAILAVIVVLV

SEQ ID NO: 332 - protein, 44 aa, Artificial Sequence
  Description of Artificial Sequence Synthetic polypeptide
ILAVIVVLVFYYLIKLKRAFGDYTSVVNRYLSLYNKYRYYSGKM

SEQ ID NO: 333 - protein, 42 aa, Betacoronavirus 1
KYRYYSGKMDTAAYREAACSQLAKAMDTFTNNNGSDVLYQPP

SEQ ID NO: 334 - protein, 120 aa, Artificial Sequence
  Description of Artificial Sequence Synthetic polypeptide
SADGVQCYTPHSQISYSNFYASGCVLSSACTMFTMADGSPQPYCYSLVPHVRYNLANAKGFIRFPEVLREGLVRIVRTRSMSYCRVGLCEEADEGICFNFNGSWVLNNDYYRSLPGTFCG

SEQ ID NO: 335 - protein, 119 aa, Artificial Sequence
  Description of Artificial Sequence Synthetic polypeptide
YYRSLPGTFCGRDVFDLIYQLFKGLAQPVDFLALTASSIAGAILAVIVVLVFYYLIKLKRAFGDYTSVVNRYLSLYNKYRYYSGKMDTAAYREAACSQLAKAMDTFTNNNGSDVLYQPP

SEQ ID NO: 336 - protein, 228 aa, Artificial Sequence
  Description of Artificial Sequence Synthetic polypeptide
SADGVQCYTPHSQISYSNFYASGCVLSSACTMFTMADGSPQPYCYSLVPHVRYNLANAKGFIRFPEVLREGLVRIVRTRSMSYCRVGLCEEADEGICFNFNGSWVLNNDYYRSLPGTFCGRDVFDLIYQLFKGLAQPVDFLALTASSIAGAILAVIVVLVFYYLIKLKRAFGDYTSVVNRYLSLYNKYRYYSGKMDTAAYREAACSQLAKAMDTFTNNNGSDVLYQPP

SEQ ID NO: 337 - protein, 21 aa, Betacoronavirus 1
SLAMLLVKHKHLYLTMYITPV

SEQ ID NO: 338 - protein, 21 aa, Betacoronavirus 1
LYLTMYITPVLFTLLYNNYLV

SEQ ID NO: 339 - protein, 28 aa, Betacoronavirus 1
IKIVLLCYLFIGYIISCYWGLFSLMNSL

SEQ ID NO: 340 - protein, 29 aa, Betacoronavirus 1
WGLFSLMNSLFRMPLGVYNYKISVQELRY

SEQ ID NO: 341 - protein, 28 aa, Betacoronavirus 1
YKISVQELRYMNANGLRPPKNSFEALML

SEQ ID NO: 342 - protein, 29 aa, Betacoronavirus 1
PKNSFEALMLNFKLLGIGGVPIIEVSQFQ

SEQ ID NO: 343 - protein, 32 aa, Betacoronavirus 1
SLAMLLVKHKHLYLTMYITPVLFTLLYNNYLV

SEQ ID NO: 344 - protein, 47 aa, Betacoronavirus 1
IKIVLLCYLFIGYIISCYWGLFSLMNSLFRMPLGVYNYKISVQELRY

SEQ ID NO: 345 - protein, 47 aa, Betacoronavirus 1
YKISVQELRYMNANGLRPPKNSFEALMLNFKLLGIGGVPIIEVSQFQ

SEQ ID NO: 346 - protein, 116 aa, Artificial Sequence
  Description of Artificial Sequence Synthetic polypeptide
SLAMLLVKHKHLYLTMYITPVLFTLLYNNYLVIKIVLLCYLFIGYIISCYWGLFSLMNSLFRMPLGVYNYKISVQELRYMNANGLRPPKNSFEALMLNFKLLGIGGVPIIEVSQFQ

SEQ ID NO: 347 - protein, 26 aa, Betacoronavirus 1
SKLTDVKCANVVLLNCLQHLHVASNS

SEQ ID NO: 348 - protein, 26 aa, Betacoronavirus 1
LQHLHVASNSKLWHYCSTLHNEILAT

SEQ ID NO: 349 - protein, 26 aa, Betacoronavirus 1
STLHNEILATSDLSVAFEKLAQLLIV

SEQ ID NO: 350 - protein, 26 aa, Betacoronavirus 1
FEKLAQLLIVLFANPAAVDSKCLTSI

SEQ ID NO: 351 - protein, 25 aa, Betacoronavirus 1
AVDSKCLTSIEEVCDDYAKDNTVLQ

SEQ ID NO: 352 - protein, 39 aa, Betacoronavirus 1
SKLTDVKCANVVLLNCLQHLHVASNSKLWHYCSTLHNEI

SEQ ID NO: 353 - protein, 38 aa, Betacoronavirus 1
HYCSTLHNEILATSDLSVAFEKLAQLLIVLFANPAAVD

SEQ ID NO: 354 - protein, 89 aa, Betacoronavirus 1
SKLTDVKCANVVLLNCLQHLHVASNSKLWHYCSTLHNEILATSDLSVAFEKLAQLLIVLFANPAAVDSKCLTSIEEVCDDYAKDNTVLQ

SEQ ID NO: 355 - protein, 31 aa, Betacoronavirus 1
YFTKKDWYDFVENPDIINVYKKLGPIFNRAL

SEQ ID NO: 356 - protein, 30 aa, Betacoronavirus 1
KLGPIFNRALVSATEFADKLVEVGLVGVLT

SEQ ID NO: 357 - protein, 29 aa, Betacoronavirus 1
VEVGLVGVLTLDNQDLNGKWYDFGDYVIA

SEQ ID NO: 358 - protein, 31 aa, Betacoronavirus 1
WYDFGDYVIAAPGCGVAIADSYYSYIMPMLT

SEQ ID NO: 359 - protein, 30 aa, Betacoronavirus 1
DLVQYDFTDYKLELFNKYFKHWSMPYHPNT

SEQ ID NO: 360 - protein, 30 aa, Betacoronavirus 1
HWSMPYHPNTVDCQDDRCIIHCANFNILFS

SEQ ID NO: 361 - protein, 29 aa, Betacoronavirus 1
HCANFNILFSMVLPNTCFGPLVRQIFVDG

SEQ ID NO: 362 - protein, 29 aa, Betacoronavirus 1
PLVRQIFVDGVPFVVSIGYHYKELGIVMN

SEQ ID NO: 363 - protein, 29 aa, Betacoronavirus 1
HYKELGIVMNMDVDTHRYRLSLKDLLLYA

SEQ ID NO: 364 - protein, 50 aa, Betacoronavirus 1
YFTKKDWYDFVENPDIINVYKKLGPIFNRALVSATEFADKLVEVGLVGVL

SEQ ID NO: 365 - protein, 51 aa, Betacoronavirus 1
LVEVGLVGVLTLDNQDLNGKWYDFGDYVIAAPGCGVAIADSYYSYIMPMLT

SEQ ID NO: 366 - protein, 42 aa, Betacoronavirus 1
DLVQYDFTDYKLELFNKYFKHWSMPYHPNTVDCQDDRCIIHC

SEQ ID NO: 367 - protein, 42 aa, Betacoronavirus 1
CQDDRCIIHCANFNILFSMVLPNTCFGPLVRQIFVDGVPFVV

SEQ ID NO: 368 - protein, 43 aa, Betacoronavirus 1
IFVDGVPFVVSIGYHYKELGIVMNMDVDTHRYRLSLKDLLLYA

SEQ ID NO: 369 - protein, 91 aa, Betacoronavirus 1
YFTKKDWYDFVENPDIINVYKKLGPIFNRALVSATEFADKLVEVGLVGVLTLDNQDLNGKWYDFGDYVIAAPGCGVAIADSYYSYIMPMLT

SEQ ID NO: 370 - protein, 107 aa, Betacoronavirus 1
DLVQYDFTDYKLELFNKYFKHWSMPYHPNTVDCQDDRCIIHCANFNILFSMVLPNTCFGPLVRQIFVDGVPFVVSIGYHYKELGIVMNMDVDTHRYRLSLKDLLLYA

SEQ ID NO: 371 - protein, 198 aa, Artificial Sequence
  Description of Artificial Sequence Synthetic polypeptide
YFTKKDWYDFVENPDIINVYKKLGPIFNRALVSATEFADKLVEVGLVGVLTLDNQDLNGKWYDFGDYVIAAPGCGVAIADSYYSYIMPMLTDLVQYDFTDYKLELFNKYFKHWSMPYHPNTVDCQDDRCIIHCANFNILFSMVLPNTCFGPLVRQIFVDGVPFVVSIGYHYKELGIVMNMDVDTHRYRLSLKDLLLYA

SEQ ID NO: 372 - protein, 28 aa, Betacoronavirus 1
AYTKRNVLPTLTQMNLKYAISAKNRART

SEQ ID NO: 373 - protein, 28 aa, Betacoronavirus 1
AISAKNRARTVAGVSILSTMTGRMFHQK

SEQ ID NO: 374 - protein, 28 aa, Betacoronavirus 1
TMTGRMFHQKCLKSIAATRGVPVVIGTT

SEQ ID NO: 375 - protein, 28 aa, Betacoronavirus 1
RGVPVVIGTTKFYGGWDDMLRRLIKDVD

SEQ ID NO: 376 - protein, 28 aa, Betacoronavirus 1
MLRRLIKDVDNPVLMGWDYPKCDRAMPN

SEQ ID NO: 377 - protein, 29 aa, Betacoronavirus 1
YPKCDRAMPNLLRIVSSLVLARKHETCCS

SEQ ID NO: 378 - protein, 28 aa, Betacoronavirus 1
LARKHETCCSQSDRFYRLANECAQVLSE

SEQ ID NO: 379 - protein, 28 aa, Betacoronavirus 1
ANECAQVLSEIVMCGGCYYVKPGGTSSG

SEQ ID NO: 380 - protein, 29 aa, Betacoronavirus 1
YVKPGGTSSGDATTAFANSVFNICQAVSA

SEQ ID NO: 381 - protein, 28 aa, Betacoronavirus 1
VFNICQAVSANVCALMSCNGNKIEDLSI

SEQ ID NO: 382 - protein, 28 aa, Betacoronavirus 1
NGNKIEDLSIRALQKRLYSHVYRSDKVD

SEQ ID NO: 383 - protein, 28 aa, Betacoronavirus 1
SHVYRSDKVDSTFVTEYYEFLNKHFSMM

SEQ ID NO: 384 - protein, 29 aa, Betacoronavirus 1
EFLNKHFSMMILSDDGVVCYNSDYASKGY

SEQ ID NO: 385 - protein, 29 aa, Betacoronavirus 1
YNSDYASKGYIANISAFQQVLYYQNNVFM

SEQ ID NO: 386 - protein, 28 aa, Betacoronavirus 1
VLYYQNNVFMSESKCWVEHDINNGPHEF

SEQ ID NO: 387 - protein, 29 aa, Betacoronavirus 1
HDINNGPHEFCSQHTMLVKMDGDDVYLPY

SEQ ID NO: 388 - protein, 29 aa, Betacoronavirus 1
MDGDDVYLPYPNPSRILGAGCFVDDLLKT

SEQ ID NO: 389 - protein, 29 aa, Betacoronavirus 1
GCFVDDLLKTDSVLLIERFVSLAIDAYPL

SEQ ID NO: 390 - protein, 47 aa, Betacoronavirus 1
AYTKRNVLPTLTQMNLKYAISAKNRARTVAGVSILSTMTGRMFHQKC

SEQ ID NO: 391 - protein, 47 aa, Betacoronavirus 1
MTGRMFHQKCLKSIAATRGVPVVIGTTKFYGGWDDMLRRLIKDVDNP

SEQ ID NO: 392 - protein, 46 aa, Betacoronavirus 1
RRLIKDVDNPVLMGWDYPKCDRAMPNLLRIVSSLVLARKHETCCSQ

SEQ ID NO: 393 - protein, 46 aa, Betacoronavirus 1
ARKHETCCSQSDRFYRLANECAQVLSEIVMCGGCYYVKPGGTSSGD

SEQ ID NO: 394 - protein, 46 aa, Betacoronavirus 1
VKPGGTSSGDATTAFANSVFNICQAVSANVCALMSCNGNKIEDLSI

SEQ ID NO: 395 - protein, 46 aa, Betacoronavirus 1
NGNKIEDLSIRALQKRLYSHVYRSDKVDSTFVTEYYEFLNKHFSMM

SEQ ID NO: 396 - protein, 47 aa, Betacoronavirus 1
EFLNKHFSMMILSDDGVVCYNSDYASKGYIANISAFQQVLYYQNNVF

SEQ ID NO: 397 - protein, 47 aa, Betacoronavirus 1
QVLYYQNNVFMSESKCWVEHDINNGPHEFCSQHTMLVKMDGDDVYLP

SEQ ID NO: 398 - protein, 49 aa, Betacoronavirus 1
KMDGDDVYLPYPNPSRILGAGCFVDDLLKTDSVLLIERFVSLAIDAYPL

SEQ ID NO: 399 - protein, 125 aa, Betacoronavirus 1
AYTKRNVLPTLTQMNLKYAISAKNRARTVAGVSILSTMTGRMFHQKCLKSIAATRGVPVVIGTTKFYGGWDDMLRRLIKDVDNPVLMGWDYPKCDRAMPNLLRIVSSLVLARKHETCCSQSDRFY

SEQ ID NO: 400 - protein, 125 aa, Betacoronavirus 1
ETCCSQSDRFYRLANECAQVLSEIVMCGGCYYVKPGGTSSGDATTAFANSVFNICQAVSANVCALMSCNGNKIEDLSIRALQKRLYSHVYRSDKVDSTFVTEYYEFLNKHFSMMILSDDGVVCYN

SEQ ID NO: 401 - protein, 113 aa, Betacoronavirus 1
ILSDDGVVCYNSDYASKGYIANISAFQQVLYYQNNVFMSESKCWVEHDINNGPHEFCSQHTMLVKMDGDDVYLPYPNPSRILGAGCFVDDLLKTDSVLLIERFVSLAIDAYPL

SEQ ID NO: 402 - protein, 341 aa, Betacoronavirus 1
AYTKRNVLPTLTQMNLKYAISAKNRARTVAGVSILSTMTGRMFHQKCLKSIAATRGVPVVIGTTKFYGGWDDMLRRLIKDVDNPVLMGWDYPKCDRAMPNLLRIVSSLVLARKHETCCSQSDRFYRLANECAQVLSEIVMCGGCYYVKPGGTSSGDATTAFANSVFNICQAVSANVCALMSCNGNKIEDLSIRALQKRLYSHVYRSDKVDSTFVTEYYEFLNKHFSMMILSDDGVVCYNSDYASKGYIANISAFQQVLYYQNNVFMSESKCWVEHDINNGPHEFCSQHTMLVKMDGDDVYLPYPNPSRILGAGCFVDDLLKTDSVLLIERFVSLAIDAYPL

SEQ ID NO: 403 - protein, 120 aa, Unknown
  Description of Unknown Bovine papillomavirus (BPV) sequence
SLVSTDNQIFNRPYWLFRAQGMNNGIAWNNLLFLTVGDNTRGTNLTISVASDGTPLTEYDSSKFNVYHRHMEEYKLAFILELCSVEITAQTVSHLQGLMPSVLENWEIGVQPPTSSILED

SEQ ID NO: 404 - protein, 118 aa, Unknown
  Description of Unknown BPV isolate NY8385 sequence
SLVTSSNQLFNKPYWIRRAQGNNNGILWGNQCFVTVGDTTRGTNFDISVPTAGGTQYKATEFNQYKRHVEEYELQFVLQACVVRLTPETVAHLYQMDSRILDDWNLGVVTSQNGALAE

SEQ ID NO: 405 - protein, 212 aa, Severe acute respiratory syndrome coronavirus 2
TVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALL

SEQ ID NO: 406 - protein, 213 aa, Betacoronavirus 1
TADEAIKFLKEWNFSLGIILLFITIILQFGYTSRSMFVYVIKMIILWLMWPLTIILTIFNCVYALNNVYLGLSIVFTIVAIIMWIVYFVNSIRLFIRTGSFWSFNPETNNLMCIDMKGTMYVRPIIEDYHTLTVTIIRGHLYIQGIKLGTGYSLADLPAYMTVAKVTHLCTYKRGFLDRISDTSGFAVYVKSKVGNYRLPSTQKGSGMDTALL

SEQ ID NO: 407 - protein, 214 aa, Human coronavirus HKU1
FTSDQAVTFLKEWNFSLGVILLFITIILQFGYTSRSMFVYLIKMIILWLMWPLTITLTIFNCFYALNNAFLAFSIVFTIISIVIWILYFVNSIRLFIRTGSWWSFNPETNNLMCIDMKGKMFVRPVIEDYHTLTATVIRGHLYIQGVKLGTGYTLSDLPVYVTVAKVQVLCTYKRAFLDKLDVNSGFAVFVKSKVGNYRLPSSKPSGMDTALLR

SEQ ID NO: 408 - protein, 215 aa, Betacoronavirus 1
WTADEAIKFLKEWNFSLGIILLFITIILQFGYTSRSMFVYVIKMIILWLMWPLTIILTIFNCVYALNNVYLGLSIVFTIVAIIMWIVYFVNSIRLFIRTGSFWSFNPETNNLMCIDMKGTMYVRPIIEDYHTLTVTIIRGHLYIQGIKLGTGYSLADLPAYMTVAKVTHLCTYKRGFLDRISDTSGFAVYVKSKVGNYRLPSTQKGSGMDTALLR

SEQ ID NO: 409 - protein, 269 aa, Betacoronavirus 1
GYWYRHNRRSFKTADGNQRQLLPRWYFYYLGTGPHAKDQYGDIDGVYWVASNQADVNTPADIVDRDPSSDEAIPTRFPPGTVLPQGYYIEGSGRSAPNSRSTSRTSSRASSAGSRSRANSGNRTPTSGVTPDMADQIASLVLAKLGKDATKPQQVTKHTAKEVRQKPRQKRSPNKQCTVQQCFGKRGPNQNFGGGEMLKLGTSDPQFPILAELAPTAGAFFFGSRLELAKVQNLSGNELRYNGAIRFDSTLSGFETIMKVLNENLNAYQ

SEQ ID NO: 410 - protein, 273 aa, Severe acute respiratory syndrome coronavirus 2
GYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALADRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYK

SEQ ID NO: 411 - protein, 24 aa, Severe acute respiratory syndrome coronavirus 2
SWFTALTQHGKEDLKFPRGQGVPI

SEQ ID NO: 412 - protein, 40 aa, Betacoronavirus 1
PHAKDQYGTDIDGVYWVASNQADVNTPADIVDRDPSSDEA

SEQ ID NO: 413 - protein, 40 aa, Human coronavirus HKU1; identical to SEQ ID NO: 261

SEQ ID NO: 414 - protein, 32 aa, Betacoronavirus 1
RSTSRTSSRASSAGSRSRANSGNRTPTSGVTP

SEQ ID NO: 415 - protein, 32 aa, Human coronavirus HKU1; identical to SEQ ID NO: 251

SEQ ID NO: 416 - protein, 15 aa, Betacoronavirus 1
SRLELAKVQNLSGNP

SEQ ID NO: 417 - protein, 15 aa, Human coronavirus HKU1
SKLDLVKRDSEADSP

SEQ ID NO: 418 - protein, 256 aa, Betacoronavirus 1
SKASSRSAIEDLLFDKVKLSDVGFVEAYNNCTGGAEIRDLICVQSYKGIKVLPPLLSENQISGYTLAATSASLFPPWTAAAGVPFYLNVQYRINGLGVTMDVLSQNQKLIANAFNNALYAIQEGFDATNSALVKIQAVVNANAEALNNLLQQLSNRFGAISASLQEILSRLDALEAEAQIDRLINGRLTALNAYVSQQLSDSTLVKFSAAQAMEKVNECVKSQSSRINFCGNGNHIISLVQNAPYGLYFIHFSYVP

SEQ ID NO: 419 - protein, 260 aa, Severe acute respiratory syndrome coronavirus 2
SKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVP

SEQ ID NO: 420 - protein, 75 aa, Betacoronavirus 1
PNLPDFKEELDQWFKNQTSVAPDLSLDYINVTFLDLQVEMNRLQEAIKVLNQSYINLKDIGTYEYYVKWPWYVWL

SEQ ID NO: 421 - protein, 76 aa, Severe acute respiratory syndrome coronavirus 2
PELDSFKEELDKYFKNHTSPDVDLGDISGINASVVNIQKEIDRLNEVAKNLNESLIDLQELGKYEQYIKWPWYIWL

SEQ ID NO: 422 - protein, 73 aa, Betacoronavirus 1
PNLPDFKEELDQWFKNQTSVAPDLSLDYINVTFLDLQVEMNRLQEAIKVLNQSYINLKDIGTYEYYVKWPWYV

SEQ ID NO: 423 - protein, 75 aa, Human coronavirus HKU1
PKLSDFESELSHWFKNQTSIAPNLTLNLHTINATFLDLYYEMNLIQESIKSLNNSYINLKDIGTYEMYVKWPWYV

SEQ ID NO: 424 - protein, 106 aa, Betacoronavirus 1
MVRMYIFFASFYYVWKSYVHVVDGCNSSTCMMCYKRNRATRVECTTIVNGVRRSFYVYANGGKGFCKLHNWNCVNCDTFCAGSTFISDEVARDLSLQFKRPINPTD

SEQ ID NO: 425 - protein, 106 aa, Severe acute respiratory syndrome coronavirus 2; identical to SEQ ID NO: 313

SEQ ID NO: 426 - protein, 61 aa, Betacoronavirus 1
NAAVFYAQSLFRPILMVDKNLITTANTGTSVTETMFDVYVDTFLSMFDVDKKSLNALIATA

SEQ ID NO: 427 - protein, 61 aa, Severe acute respiratory syndrome coronavirus 2
SASVYYSQLMCQPILLLDQALVSDVGDSAEVAVKMFDAYVNTFSSTFNVPMEKLKTLVATA

SEQ ID NO: 428 - protein, 55 aa, Betacoronavirus 1
ELTDESCNNLVPTYLKSDNIVAADLGVLIQNSAKHVQGNVAKIAGVSCIWSVDAF

SEQ ID NO: 429 - protein, 55 aa, Severe acute respiratory syndrome coronavirus 2
EVTGDSCNNYMLTYNKVENMTPRDLGACIDCSARHINAQVAKSHNIALIWNVKDF

SEQ ID NO: 430 - protein, 45 aa, Severe acute respiratory syndrome coronavirus 2
SAVGNICYTPSKLIEYTDFATSACVLAAECTIFKDASGKPVPYCY

SEQ ID NO: 431 - protein, 133 aa, Betacoronavirus 1
SLVPHVRYNLANAKGFIRFPEVLREGLVRIVRTRSMSYCRVGLCEEADEGICFNFNGSWVLNNDYYRSLPGTFCGRDVFDLIYQLFKGLAQPVDFLALTASSIAGAILAVIVVLVFYYLIKLKRAFGDYTSVV

SEQ ID NO: 432 - protein, 132 aa, Severe acute respiratory syndrome coronavirus 2
SLRPDTRYVLMDGSIIQFPNTYLEGSVRVVTTFDSEYCRHGTCERSEAGVCVSTSGRWVLNNDYYRSLPGVFCGVDAVNLLTNMFTPLIQPIGALDISASIVAGGIVAIVVTCLAYYFMRFRRAFGEYSHVV

SEQ ID NO: 433 - protein, 50 aa, Betacoronavirus 1
NRYLSLYNKYRYYSGKMDTAAYREAACSQLAKAMDTFTNNNGSDVLYQPP

SEQ ID NO: 434 - protein, 49 aa, Severe acute respiratory syndrome coronavirus 2
NRYLALYNKYKYFSGAMDTTSYREAACCHLAKALNDFSNSGSDVLYQPP

SEQ ID NO: 435 - protein, 84 aa, Betacoronavirus 1
IKIVLLCYLFIGYIISCYWGLFSLMNSLFRMPLGVYNYKISVQELRYMNANGLRPPKNSFEALMLNFKLLGIGGVPIIEVSQFQ

SEQ ID NO: 436 - protein, 84 aa, Severe acute respiratory syndrome coronavirus 2
LQCIMLVYCFLGYFCTCYFGLFCLLNRYFRLTLGVYDYLVSTQEFRYMNSQGLLPPKNSIDAFKLNIKLLGVGGKPCIKVATVQ

SEQ ID NO: 437 - protein, 31 aa, Betacoronavirus 1
SLAMLLVKHKHLYLTMYITPVLFTLLYNNYL

SEQ ID NO: 438 - protein, 31 aa, Severe acute respiratory syndrome coronavirus 2
AFAMMFVKHKHAFLCLFLLPSLATVAYFNMV

SEQ ID NO: 439 - protein, 67 aa, Betacoronavirus 1
SKLTDVKCANVVLLNCLQHLHVASNSKLWHYCSTLHNEILATSDLSVAFEKLAQLLIVLFANPAAVD

SEQ ID NO: 440 - protein, 67 aa, Severe acute respiratory syndrome coronavirus 2
SKMSDVKCTSVVLLSVLQQLRVESSSKLWAQCVQLHNDILLAKDTTEAFEKMVSLLSVLLSMQGAVD

SEQ ID NO: 441 - protein, 495 aa, Deltapapillomavirus 4
MALWQQGQKLYLPPTPVSKVLCSETYVQRKSIFYHAETERLLTIGHPYYPVSIGAKTVPKVSANQYRVFKIQLPDPNQFALPDRTVHNPSKERLVWAVIGVQVSRGQPLGGTVTGHPTFNALLDAENVNRKVTTQTTDDRKQTGLDAKQQQILLLGCTPAEGEYWTTARPCVTDRLENGACPPLELKNKHIEDGDMMEIGFGAANFKEINASKSDLPLDIQNEICLYPDYLKMAEDAAGNSMFFFARKEQVYVRHIWTRGGSEKEAPTTDFYLKNNKGDATLKIPSVHFGSPSGSLVSTDNQIFNRPYWLFRAQGMNNGIAWNNLLFLTVGDNTRGTNLTISVASDGTPLTEYDSSKFNVYHRHMEEYKLAFILELCSVEITAQTVSHLQGLMPSVLENWEIGVQPPTSSILEDTYRYIESPATKCASNVIPAKEDPYAGFKFWNIDLKEKLSLDLDQFPLGRRFLAQQGAGCSTVRKRRISQKTSSKPAKKKKK

SEQ ID NO: 442 - protein, 91 aa, Severe acute respiratory syndrome coronavirus 2
YFNKKDWYDFVENPDILRVYANLGERVRQALLKTVQFCDAMRNAGIVGVLTLDNQDLNGNWYDFGDFIQTTPGSGVPVVDSYYSLLMPILT

SEQ ID NO: 443 - protein, 107 aa, Severe acute respiratory syndrome coronavirus 2
DLLKYDFTEERLKLFDRYFKYWDQTYHPNCVNCLDDRCILHCANFNVLFSTVFPPTSFGPLVRKIFVDGVPFVVSTGYHFRELGVVHNQDVNLHSSRLSFKELLVYA

SEQ ID NO: 444 - protein, 341 aa, Severe acute respiratory syndrome coronavirus 2
AYTKRNVIPTITQMNLKYAISAKNRARTVAGVSICSTMTNRQFHQKLLKSIAATRGATVVIGTSKFYGGWHNMLKTVYSDVENPHLMGWDYPKCDRAMPNMLRIMASLVLARKHTTCCSLSHRFYRLANECAQVLSEMVMCGGSLYVKPGGTSSGDATTAYANSVFNICQAVTANVNALLSTDGNKIADKYVRNLQHRLYECLYRNRDVDTDFVNEFYAYLRKHFSMMILSDDAVVCFNSTYASQGLVASIKNFKSVLYYQNNVFMSEAKCWTETDLTKGPHEFCSQHTMLVKQGDDYVYLPYPDPSRILGAGCFVDDIVKTDGTLMIERFVSLAIDAYPL